(19) United States
(12) Patent Application Publication
(10) Pub. No.: US 2023/0167157 A1
(43) Pub. Date: Jun. 1, 2023

(54) ANTIGENIC DETERMINANTS PROTECTIVE IMMUNITY, SERODIAGNOSTIC AND MULTIVALENT SUBUNITS PRECISION VACCINE AGAINST SARS-CoV-2

(71) Applicant: ARABIAN GULF UNIVERSITY, Manama (BH)

(72) Inventors: Mohamed-Dahmani FATHALLAH, Manama (BH); Khaled TRABELSI, Manama (BH); Noureddine BEN KHALAF, Manama (BH); Ahmad Rasmi RAMADAN, Manama (BH)

(73) Assignee: ARABIAN GULF UNIVERSITY, Manama (BH)

(21) Appl. No.: 18/058,403

(22) Filed: Nov. 23, 2022

Related U.S. Application Data
(60) Provisional application No. 63/283,835, filed on Nov. 29, 2021.

(51) Int. Cl. C07K 14/005; C12N 7/00; A61K 39/215; A61P 31/14

(52) U.S. Cl. CPC C07K 14/005; C12N 7/00; A61K 39/215; A61P 31/14; C12N 2770/20022; C12N 2770/20034; C07K 2319/50; A61K 2039/625

(57) ABSTRACT

Immunogenic peptides, polypeptides, protein subunits, nucleic acids encoding the same, compositions and vaccines containing the immunogenic peptides or nucleic acids, and methods of vaccination and/or treatment of SARS-CoV-2 infection. A serological diagnostic assay as well as a use of the serological assay with peptide P3 (SEQ ID NO: 3) to reveal whether a human suspected to be infected or to have been infected by a SARS-CoV-2 virus has generated an anti SARS-CoV-2 protective immunity,

### CROSS REFERENCE TO RELATED APPLICATIONS

This application claims priority to U.S. Provisional Application No. 63/283,835, filed Nov. 29, 2021 which is incorpor ated by reference for all purposes, The description and disclosure of a PCT application being filed on the same day as this U.S. application are also incorpor ated by reference for all purposes.

### REFERENCE TO A SEQUENCE LISTING

In accordance with 37 CFR § 1.52(e)(5), the present specification refers to a Sequence Listing submitted electronically as a .xml file named “543809US_ST26” The .xml file was generated on Nov. 23, 2022 and is 238,251 bytes in size. The entire contents of the. Sequence Listing are hereby incorpor ated by reference.

### BACKGROUND OF THE INVENTION

### Field of the Invention

The present invention pertains to the fields of virology and immunology especially to polypeptide antigens comprising epitopes of SARS-CoV-2 and to nucleic acids encoding them.

### Description of Related Art

On January 2020, the World Health Organization (WHO) declared the new coronavirus outbreak and its subsequent infectious disease COVID-19 epidemic and a public health emergency. International concern was raised when the epidemic turned into a pandemic which has now swept the world. The pandemic has affected more than 600 million people, claimed close to 7 million lives, and has jeopardized human social and economic life globally; Elflein, 2020, Roser al., 2020, GISAID 2021. A new coronavirus was identified as the etiologic agent of the Severe Acute Respiratory Syndrome Disease 2019 (COVID-19) and was named SARS-CoV-2.

SARS-CoV-2 is a new member of the Coronaviridae family (CoVs) which contains four genera of CoVs, namely, Alphacoronavirus (αCoV), Betucoronavirus (βCoV), Deltacoronavirus (SCoV), and Gammacoronavirus (γCoV), These viruses are of zoonotic origin with αCoV and βCoV found in bats and rodents while δCoV and γCoV are1 encountered in avian species, SARS-CoV-2 belongs to the genus beatacoronavirus (Siddell et 1983).

Human transmission of SARS-CoV-2 and COVID-19 occurs through contact, droplets, and fomites and the common clinical symptoms are fever, cough, muscle aches, headache, and diarrhea; Sheleme, et al., 2020. The principal clinical feature of the severe disease is acute onset of hypoxemic respiratory failure. with bilateral infiltrates; Verity, et al., 2020. Nevertheless, the disease course can vary from asymptomatic, mild, moderate and severe to critical forms; see NIH COVID treatment guidelines 2021, Woelfel et al. 2020, G. Chen at al., 2020, Oran et al. 2020.

Furthermore, while the spreading of the SARS-CoV-2 virus is global, the prevalence/incidence of the disease varies throughout the world, for some regions being more severely hit then others; Dawood et., 2020. Even the overall case fatality ratio of COVID-19 varies between location and intensity of transmission; Sorci,et al. 2020. Such discrepancies may be due to several factors that affect the virus infectious potency (infectivity) among which the health care prevention system orgartization and the stringency of the confinement measures, as well as the economic differences.

Nevertheless, other important factors intrinsic to the virus structure and the host genetic background are crucial determinants of the SARS-CoV-2 infectivity. Doubtlessly, the uttermost determinant of the COVID19 pandemic is that people are not equal before the severity of COVE-19. Indeed, the disease course differs with age, gender, ethnicity, underlying clinical conditions and virus variants Ghisolfi et al., 2020. Other diseases modifying factors are associated. with genetic traits such as those driving the immune response, the blood groups, the coagulation system and the ACE2 receptor variants.

The SARS-CoV-2 virus genome was rapidly sequenced which spurred the development of diagnostic tests and active research into vaccines and therapeutics,

Virus structure. SARS-COV-2 virus is an enveloped, positive-sense, single-stranded RNA virus. it is a spherical to pleomorphic particle, measuring between 80 and 180 nm in length. SARS CoV-2 contains four structural proteins, namely envelope (E), spike (S), membrane (M), and nucleocapsid (N) (Malik, 2020).

The S, M, and E proteins together form. the envelope of the virus. The M protein is the most abundant, mostly responsible for the shape of the envelope. The E protein is the smallest structural protein. The S and M are transmembrane proteins involved M virus assembly during replication. The N proteins remain associated with the RNA forming a nucleocapsid inside the envelope. Although the N protein is largely involved in processes relating to the viral genome, it is also involved in other aspects of the CoV replication cycle (e.g., assembly and budding) and the host cellular response to viral infection. Polymers of the S proteins remain embedded in the envelope giving it a crown-like appearance, thus the name coronavirus.

The SARS-CoV-2 gnome is about 30 kb (29,891 nucleotides) and has an overall G+C content of 38%. It encodes about 9,360 amino acids.

The genomic sequence of the original SARS-CoV-2 isolated in Wuhun, China the reference sequence) is closer to the SARS-like bat CoVs genome. This genorne contains 12 functional open reading frames (OREs) (Chan et al., 2020), nine transcription-regulatory sequences, nine sub genomic mRNAs with terminal untranslated regions and a conserved leader sequence.

The genome encodes 16 nonstructural proteins (nsps), 4 structural proteins, and 9 accessory proteins. The four structural proteins encoded by SARS-CoV-2 genome are S, E, M, and N, along with a number of accessory proteins that interfere with the host immune response. ORF1 is polycistronic and encodes two polyproteins, pla and pplab, and the16 non-structural proteins (NSPs). These 16 non-structural proteins encompass two cysteine proteases, namely, NSP3 (papain-like protease) and NSP5 (main protease); NSP12 (RNA-dependent RNA polymerase; and NSP13 (helicase). The other NSPs are related to transcriptional and replicanve viral functions. The remaining ORFS encode accessory and structural proteins.

The organization of the coronavirus genome comprises a 5-leader-UTR-replicase-S (Spike)-E (Enyelope)-M (Membrane)-N (Nucleocapsid)-3UTR-poly (A) tail with accessory genes interspersed within the structural genes at the 3′ end of the genome.

Mutations are observed in the NSP2 and NSP3 and the S spike protein and play a significant role in infectious capability and differentiation mechanism of SARS-CoV-2; Chan, et al, 2020.

The coronavirus spike (S) protein plays a key role in the early steps of viral infection; Walls, et at., 2020. It comprises the S1 and S2 subunits with the S1 responsible for receptor binding and the S2 mediating membrane fusion. The S1 subunit contains a signal peptide, followed by an N-terminal domain and receptor-binding domain (RBD); Lan, et al., 2020. The S1 subunit shares 70% identity with that of otter CoVs but the core receptor-binding domain (RBD) is highly conserved. The S2 subunit contains conserved a fusion peptide, heptad repeat 1 and 2, a transmembrane domain, and a cytoplasmic domain. The S2 subunit of SARS-CoV-2 is highly conserved and shares 99% identity with those of two bat SARS like CoVs and to human SARS-CoV.

Spike protein amino-acid differences are responsible for the direct interaction of spike protein with the host receptor, The spike glycoprotein binds mainly to the human ACE2 receptor present in the target cells in the respiratory tract; Yan, et al. 2020. This protein has a compact ridge that allows the virus to attach more strongly than other viruses of the same origin: Y. Zhao, et al., 2020.

After the spike protein binds with the receptor in the target cell, the viral envelope fuses with the cell membrane and releases the viral genome into the target cell. Virus entry to requires the action of the endosomal protease cathepsin L which is associated with SARS-CoV-2 infection and development of severe acute respiratory syndrome, infection by SARS-CoV can be strongly induced by trypsin treatment; Belourard, et al., 2009.

The role of proteolytic processing of the SARS-COV S protein in the activation of membrane fusion is now established and may be a turnkey into the SARS-COV viruses' infectivity. The fusion protein mechanism comprises a first pre-fusion phase where the receptor binding subunit is clamped to the fusion subunit; Belonzard, et al., 2012. Upon proteolytic activation, the receptor-binding subunit moves out of the way allowing the fusion subunit to form a pre-hairpin with target membrane via fusion peptide. Subsequently, the pre-hairpin folds back causing N and C alpha helix heptad repeats to form a 6-helix bundling pulling, the two membranes through close apposition, hemi fusion and fusion pore formation; Vankadari, 2020.

A distinguishing feature of the SARS-CoV-2 genome highlighted by comparative genomic studies, is the presence a polybasic Furin Cleavage Site (FCS) at the (S) protein S1-S2 boundary, subsequent to the insertion of 12 nucleotides encoding four amino acid residues PRRA: Coutard, 2020. A polybasic FCS does not exist in any of the coronaviruses of the same elude as SARS-COV-2.

The inserted FCS is spatially located at a random coil loop region, mostly distantly solvent-exposed; Li, 2020. The furin cleavage site at the S2 position allows trypsin-independent cell-cell fusion and strongly increases the cleavage at the S1-S2 boundary. This represents a novel priming mechanism for a viral fusion protein, in addition to the TMPRSS2 proteolytic cleavage event on the SARS-COV S protein at position 797 (S2′), acting in concert with the furin S1-S2 cleavage site to mediate membrane fusion and virus infectivity. The presence of the furin site may be associated with SARS-CoV-2 high pathogenicity of the virus; Johnson, et al., 2020.

SARS-CoV-2 invasion of human host cell is essentially but not. exclusively mediated by the interaction of the virus capsid spike protruding S protein with the Angiotensin Convertase Enzyme 2 (ACE2) (Samavati et al., 2020 Tipnis et al., 2000). ACE2 is expressed on the surface of various human body cell-types where it displays a zinc metalloproteinase activity (Albini et al, 2020). The interaction ACE2/SARS-CoV-2 occurs through the receptor-binding domain (RBD) sequence of the S1 chain of the spike protein. This functional domain is highly prone to mutations (Greaney et al., 2021). The affinity of SARS-CoV-2 for the ACE2 receptor is 10 times higher than that of SARS-CoV (Wrapp et at, 2020).

The emergence of the new coronavirus “SARS-CoV-2,” the etiologic agent of the ongoing COVID19 pandemic, urged the need for understanding the immunity developed to fight SARS-COV-2 infection and circumvent the pandemic As for the antibody-mediated immune response, it has been demonstrated that most persons infected with SARS-CoV-2 develop an antibody response 10 to 21 days post-infection while in mild cases the antibody response can take up to more than four weeks; J. Zhao et al., 2020.

Vaccines. Currently, ten different vaccines developed using conventional and new and innovative approaches are being used to face the plethor a of biological varlet), associated with SAR-CoV-2 infection. The current COVID19 vaccines work in different. ways to provide protection by conferring to the body “memory” T and B-lymphocytes that are competent to fight a SARS-CoV-2 infection. These vaccines were developed mainly using the following three different vaccination approaches:

mRNA vaccination: It is based on the use of pan of the SARS-CoV-2 RNA genetic material that gives the recipient Cells instructions for how to make a protein of the virus. The cells of the vaccinated people make copies of the protein, and eliminate the RNA molecules. The immune system recognize that protein as non-self (die antigen) and develop T and B-lymphocytes that neutralize and eliminate the virus while providing a set of immune memory cells capable of fighting the virus if a person gets infected, Pfizer/BioNtech, Moderna vaccines are examples; see Jalkanen et al., 2021: Schlake, et al, 2012; Sun et al., 2021; Polack, et al., 2021.

Vector-based vaccination: In this vaccination approach, a modified harmless version of a different Winds than SARS-COV-2 often an adenovirus is used as a viral vector. Examples include AstraZenaca, Sputnik V, Johnson & Johnson vaccines. This modified virus serves as a shell inside which genome is inserted a part of the genetic material from the SAR-CoV-2, Upon vaccination the genetic material of this viral vector gets inside the recipient cells and instructs the cells to make a protein that: is unique to the virus that causes COVID-19. This prompts the immune system to develop T and B-lymphocytes to fight. SARS-CoV-2; see Bos et al., 2020: Kremer, 2020; Mahase, 2021; Tatsis & Ertl, 2004; Zak et al., 2012; Zha et al., 2021; WHO 2021.

Inactivated Vaccines: An inactivated vaccine or killed vaccine is a vaccine consisting of virus particles that have been grown in cell culture, for example, in Vero cells for SARS-CoV-2, and then killed to eliminate disease producing capacity either by using heat or chemical modification of the genetic material using formalin or beta propiolactone (SARS-COV-2). in this vaccination approach, the whole viral particle is used to prime the immune system in producing immunity to the virus; see Gao et al., 2020; Iversena & Bavari, 2021; Risson, 2020; Roberts et al., 2010; Tanriover et al., 2021; Wilder-Smith & Mulholland, 2021; Z. Wu et al., 2021. Examples of inactivated vaccines include Sinopharm, Coronavac, Covacin, Sinovac.

All of COVID 19 first generation vaccines have been designed to target the original SARS-COV-2 (Wuhan strain) and tested using conventional clinical trials focusing mainly on safety and efficacy. These vaccines showed variable efficacy levels from very high to moderate and none confers sterilizing immunity. Moreover, the effectiveness of almost all COVID-19 vaccines is now challenged by the intrinsic variability of SARS-Cov-2 and the existence of the so-called variants of concern that are more transmissible with sonic that might escape the effects of specific. immunity; Krause et al., 2021.

As of September 2021, 3 variants of concern (Beta, Gamma and Delta) and 3 variants of interest (n/a, Mu and Lambda) were detected and 8 other variants are under monitoring while several other variants were de-escalated; European Center Disease Prevention & Control 2021. Therefore, mitigation efforts have to be pursued post-vaccination. Furthermore, some instances of concerning undesirable effects such as thrombocytopenia and micro blood clots, cardiac tissue inflammation and even a report of a possible reprograming of the immune response are being regularly reported; Menni et al., 2021; Thompson, et al. 2021; Trogen; et al., 2020; Föhse, et al. 2021.

Diagitostics. It was also reported that in a small number of cases IgM and Igo antibodies are not detected at all. The IgM and IgG antibodies to SARS-CoV-2 develop generally between 6-15 days post infection with a median seroconversion time of 11 to 12 days post disease onset; see Okba et al., 2020, Long et al., 2020, Belonzard et al., 2012.

The detection of IgG antibodies against SARS-CoV-2 is important in tracking the immunity to SARS-CoV-2 during the COVI19 pandemic. Indeed, IgG antibodies last longer and along with IgA antibodies are generally endowed with viral neutralizing activity, which is associated with recovery from COVID-19; Callow, et al, 1990; Wu et al., 2007:. X.iao et al., 2020; Zeng et al., 2020, Brotons et al. 2020. Therefore, the availability of serologic assays to detect antibodies against SARS-CoV-2 is a valuable tool for biologic data-based management of the SARS-CoV-2 pandemic. Indeed. it permits to know more about the immune system response particularly about the duration and protection level; Deng et al., 2020, Herzog et al. 2020, Tsaneva-Damyanova, 2020.

It is also important to evaluate the efficacy of different vaccines offered glribally. As such, it is essential to have readily available efficient assays for the identification of COVID-19 infections and for studying the immune response to SARS-CoV-2. It is also commonly admitted that serologic testing plays a role in the global response to a pandemic. Thus, an intensive development effort brought a number of both molecular and serologic assays to clinical practice. Because of the emergency of the situation, all of the developed serologic assays made commercially available display high variability in the format, the targeted antigens, the classes of antibody detected, and the specimen types. However, it is not clear how the results of these tests provide can be utilized in clinical applications, Indeed, the major American health care regulation agency FDA (Food and Drug Administration) did not require the manufacturers of serologic assays to file for any authorization but rather an emergency use authorization (EUA) was issued for the commercialization of such kits, Around a hundred of manufacturers are currently offering internally validated serologic tests commercially available.

Serologic tests are also important for the screening of patients recovered from COVID-19 for the presence of anti-SARS-COV-2 antibodies and for eligibility to be donors in convalescent plasma therapy (Bloch et al., 2020). Serologic testing, is important to identify viral antigens that elicit protective immunity and particularly those antigens that are specific to SARS-CoV-2; see Braun et al., 2020; Grifoni et al., 2020; Weiskopf et al., 2020; and Chen et al., 2021; Crooke, et al. 2020; Vashi, et al. 2020. Therefore, it is of paramount importance to develop patient-centered tests which are the most likely to help in clinical setting and risk management.

The selection of the right antigen(s) as well as the quality of antigens used for the diagnostic. and follow up of infectious diseases is crucial for the development of high quality diagnostic kits. This has been an issue since the outbreak of the COVID 19 pandemic mainly because of the emergency and limited time to develop such kits (Kevadiya et al., 2021; Li et al., 2020; Ravi, et al. 2020). The available commercial assays did not undergo thorough validation and approval process. They have several advantages and disadvantages particularly the rapid kits (Sethuraman, et al. 2020). As a result, the data currently available regarding our immune response is limited, and proper clinical utilization strategies for SARS-CoV-2 serologic assays are still needed particularly when it comes to evaluating the reinfections (Edridge, et al. 2020).

In view of the above, many controversies have arisen and many questions remain, These include whether variations in SARS-CoV-2 vaccine performance are associated with disease modifying factors described above; Whether a conventional or more innovated approach would provide a safer and more effective SARS-CoV-2 vaccine; whether to protection with a SARS-COV-2 vaccine would reduce symptoms and transmission of the virus so that lite could return to pre-pandemic status; and whether a vaccine could be engineered that provides protective immunity against a broad range of different genetic variants of SARS-CoV-2.

Consequently, there exists an urgent and critical need for effective tools for the treatment, for the identification and characterization of antigenic determinants of SARS-CoV-2 immunity, as well as serodiagnostic assays to determine the efficacy of SARS-CoV-2 vaccines.

### SUMMARY OF THE TECHNOLOGY

The technology disclosed herein pertains to peptides or polypeptides comprising SARS-Cov-2 epitopes especially those useful in therapeutic or diagnostic products. As used herein the term “peptide product” includes SARS-CoV-2 antigen complexes (e.g, quaternary complexes), whole antigens, immunogenic or antigenic fragments thereof (e.g. linear and conformational epitopes), as well as engineered or formulated products such as peptide or poly-peptide conjugates, chemically modified peptides or polypeptides biotinylated peptides), or peptide or polypeptide compositions (e.g., compositions containing two, three or more SARS-COV-2 peptides or polypeptides, adjuvants carriers, or excipients). The peptide products disclosed herein may form parts of larger protein complexes, larger protein subunits, parts of a fusion protein, parts of a chimeric protein, or parts of peptide conjugates, for example, comprising one or more epitopes of SARS-CoV-2 and a carrier. Thus, peptide products disclosed herein may form components of an immunogenic or antigenic composition that comprises one, two, three, four, five, six, seven, eight, nine, ten, or more SARS-CoV-2 epitopes.

One aspect of tins technology is directed to SARS-COV-2 antigenic determinants located at the junction of the S1/S2 subunits of the S spike protein including solvent-accessible epitopes. These epitopes have been identified and linked to protective immunity against SAILS-COV-2 and protection against COVID as well serodiagnostic products that broadly recognize multiple strains and variants of SARS-Cov-2. Peptide products having a furin cut site (PCS) or motif (PRRAR) or aTMPRSS2 cut site (or cleavage site) have features useful for development of a universal vaccine against numerous genetic variants of SARS-Cov-2 or a substantially universal serodiagnostic, The polypeptide antigens disclosed herein include, but are not limited to, those comprising peptide P3 (SEQ ID NO; 3), P3 L, (SEQ ID to NO: 43), SJ-FT (SEQ ID NO: 144), P4/TMP-Pol3 (SEQ ILS NO: 116), These two, three or four of these peptides (as or other SARS-COV-2 peptides disclosed herein) may be admixed in non-covalent form or in covalently associated together as conjugated or fused products. One tested and preferred composition contains all four of these peptides.

A related aspect of this technology is directed to polynucleotides, including natural or modified RNA or DNA, which encode the SARS-CoV-2 polypeptide antigens described herein, vectors or host cells comprising such polynucleotides as well as methods for producing SAILS-CoV-2 polypeptide antigens by expression of such polynucleotides, Such a composition can comprise polypeptide antigens targeting viral proteins in viral activation and infectivity and eliciting protective immunity.

A related aspect is directed to a composition., such as a pharmaceutical or diagnostic composition comprising. the polypeptide antigens disclosed herein, or comprising the polynucleotides encoding them, and a suitable carrier, adjuvant, of excipient.

Another aspect of this technology is directed to methods for inducing protective Immor al or cellular immune responses against SARS-CoV-2 epitopes, such as the epitopes disclosed herein. Such immune responses include antibodies that recognize SARS-CoV-2 proteins as well as T cell responses that recognize SARS-CoV-2 infected cells or processed SARS-CoV-2 antigens, This aspect also includes cells or complexes comprising SAR S-CoV-2 peptide products presented by MHC Class 1 or Class 2 molecules, such as peptides restricted to a particular allelic type of subtype of histocompatibility molecules. A practical aspect of this technology involves prophylaxis against. SARS-COV-2 infection or treatment of a SAILS-CoV-2 infection by administering the peptide products disclosed herein, or polynucleotides expressing them.

Another practical aspect of this technology directed to diagnostic methods for detecting exposure to or infection by SARS-CoV-2 or for following a SARS-COV-2 infection by detecting the peptide products disclosed herein or detecting; RNA or DNA encoding them Such methods include serodiagnostic assays detecting SARS-COV-2 epitopes that distinguish SARS-CoV-2 from other pathogenic coronaviruses.

The inventors designed or derived the peptide products disclosed herein based on antigenic, or immunogenic sequences they identified, This strategy involved focusing on epitopes that were solvent accessible at the surface of the S1RS-COV-2 S protein and particularly on epitopes that distinguish SARS-CoV-2 from other pathogenic coronaviruses. These epitopes included those within sequences that are involved in virus activation and infectivity. Investigations were first directed to the junction of the S protein subunits S1/S2 to identify peptides fulfilling these criteria. The inventors defined a sequence that contained the furin cut site (ECS motif), and which encompassed the sequences of these peptides. These peptides were used to develop an ELISA to test SARS-CoV-2 infected people for an antibody immune response. Through a serological patients-centered study, it was discovered. that the antibody response to this sequence of the SARS-CoV-2 S protein was highly associated with. protective immunity.

Similarly, the inventors defined in the S1/S2 junction, a sequence that contains the TMPRSS2 cut site and encompassed the sequences of four antigenic peptides disclosed herein. These sequences were used to develop S protein subunits that have all the features necessary for the development of a universal vaccine against SARS-Cov-2 the causative agent for COVID 19. The designed multivalent vaccine subunits deduced from the effective protective. immunity to SARS-CoV-2 have ad the potential to induce both the innate and adaptive immune systems and neutralize all the SRAS-CoV-2 genetic variants,

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained, as the same becomes better understood by reference to the following:, Figures in conjunction with the detailed description below.

The patent or application file contains at least one drawing executed in color. Copies of this patent or patent application publication with color drawing(s) will be provided by the Office upon request and payment of the necessary lee.

FIG. 1. Flow chart depicting the strategy used to identify SARS-CoV-2 antigenic determinants associated with protective immune response& The workflow and the different steps undertaken to develop the vaccine subunits are also shown,

FIGS. 2A-2F: Analysis of the SARS-CoV-2 S protein 3D model (PDB ID 6VXX). FIG. 2A. The S1/S2 Junction domain (residues 601-779) is colored in Smudge Green, the Furin protease cut site (FCS) is colored. in Salmon Red, Surface representation.

FIG. 2B. Cartoon representation and dose-up.

FIG. 2C. Surface representation of S1/S2. Junction domain solvent-exposed residues colored in cyan blue.

FIG. 2D. Surface representation. The protease TIMPRSS2 cut site is colored in Orange.

FIG. 2E. Cartoon representation and close-up.

FIG. 2F. The solvent-exposed residues at the S1/S*2 Junction domain is colored in men.

FIGS. 3A-3B: Prediction of Proteasome/Immunoproteasome MHC1/MHC2 peptides in the SARS-CoV-2 S protein S1/S2/S′2 subunits junctions.

FIG. 3A. Mapping and sequences of 14 potential antigenic peptides. Sequences overlapping or surrounding SARS-CoV-2 S protein Furin Cut Site (FCS) motif (PRRAR sequence boxed) are Shown in red and blue respectively. The core sequence of polypeptide P3 encompassing six predicted antigenic peptides used to investigate the COVID19 patient's antibody immune response by the ELISA assay, developed herein, is highlighted in green (See peptide P3 design. Table 4). The solvent exposed sequences spanning residues E648 to G654, Q662 to S676, and T683 to N696 are underlined.

FIG. 3B. Mapping and sequences of 4 potential antigenic peptides in in the S′2 junction. The sequence of peptide P4 TMP in red overlaps with SARS-CoV-2 S protein TMPRSS2 Cut Site. P3/TPM overlaps with SARS-COV-2 S protein Fusion Peptide (FP1) sequence (bold. underlined, The solvent exposed residues are highlighted in cyan blue. The IMPRSS2 cut site is boxed.

FIGS. 4A-4D. Data of the ELISA reference sera analysis,

FIG. 4A, The calculated cut off with 95% (2SD) CI is equal to an OD of 0.450.

FIG. 48. Data obtained with negative reference sera from the NIBSC (UK).

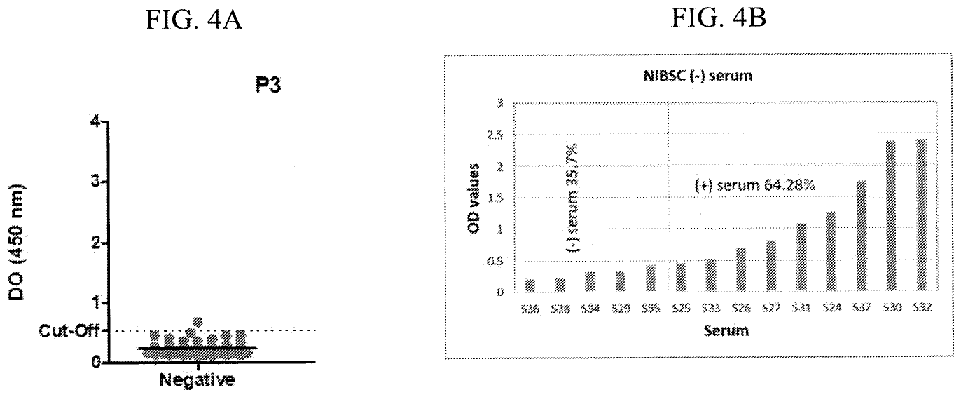

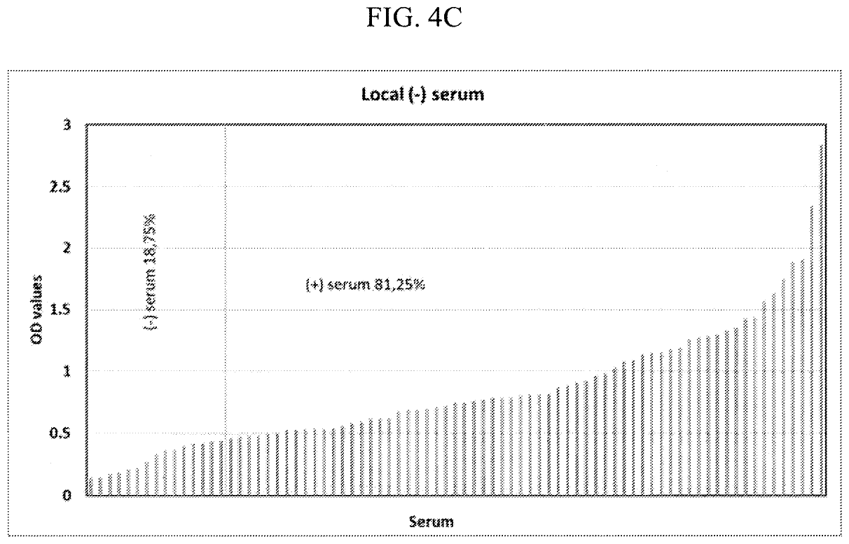

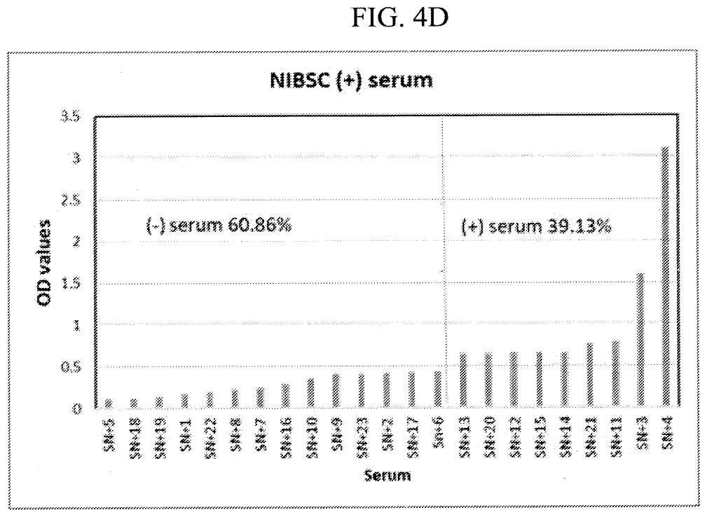

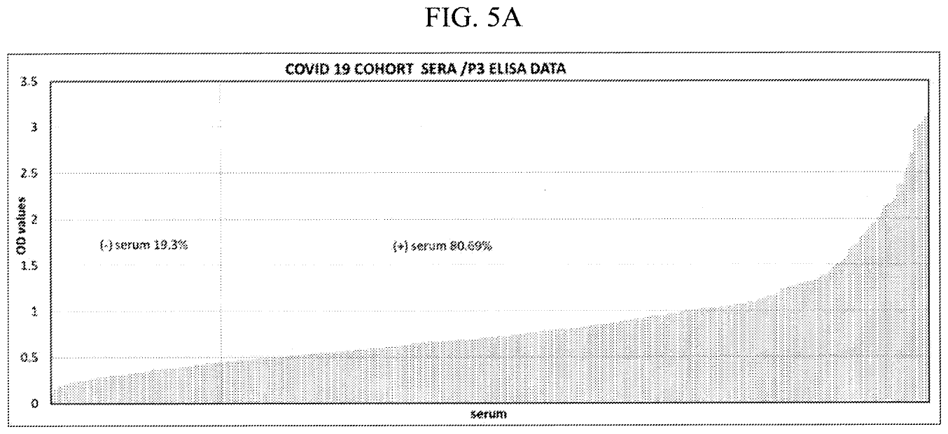

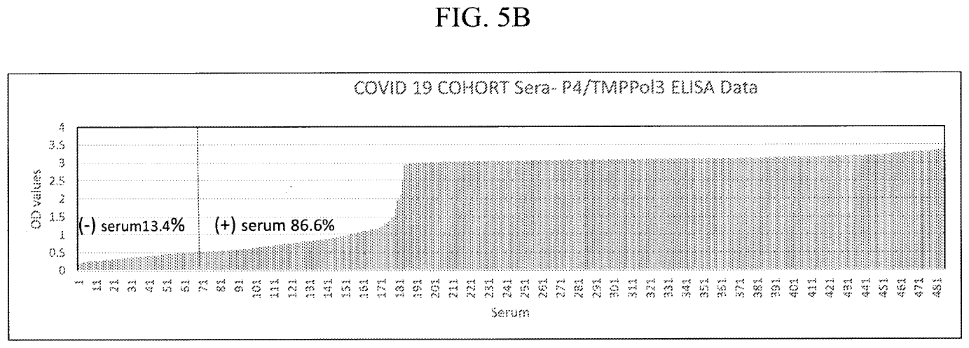

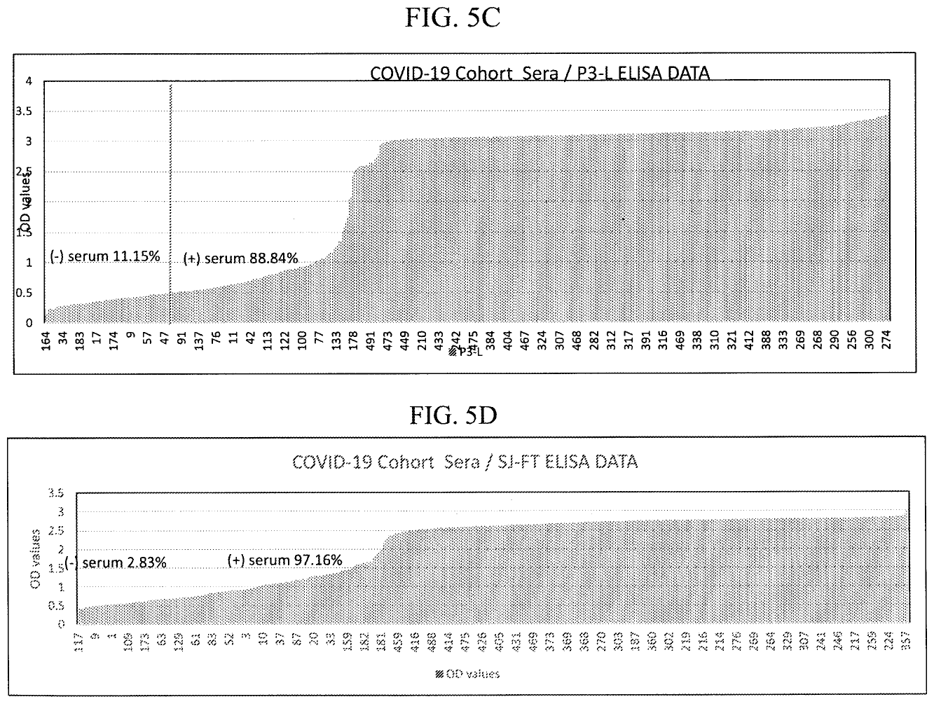

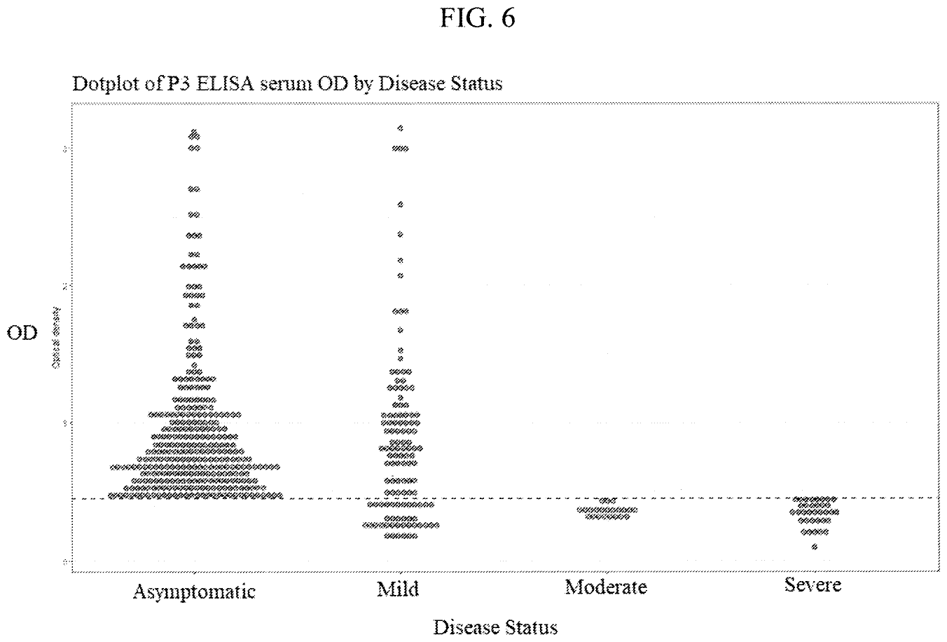

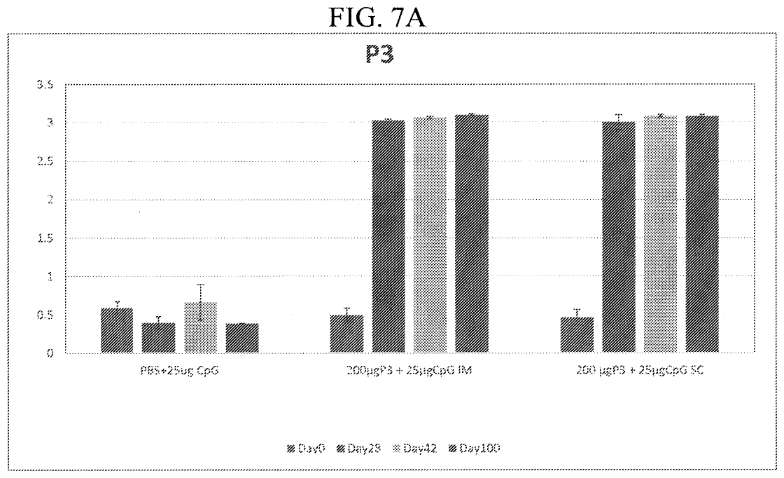

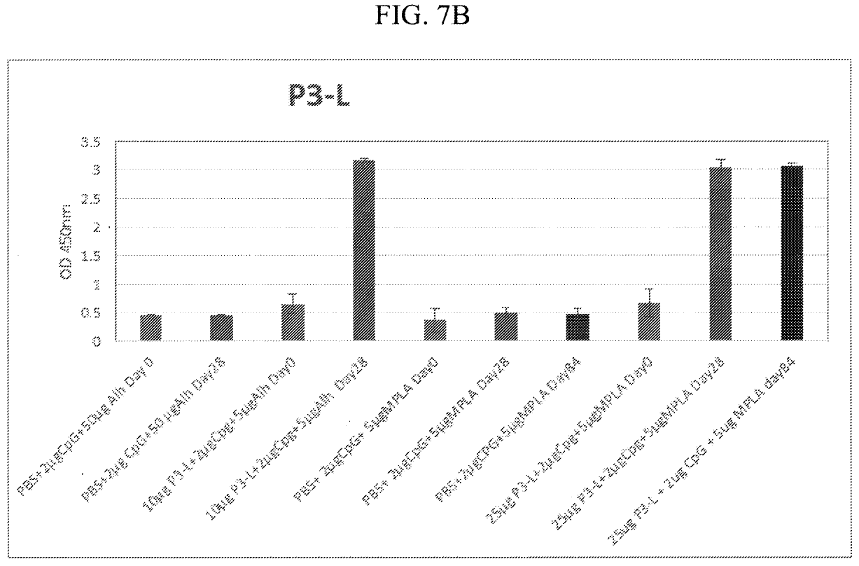

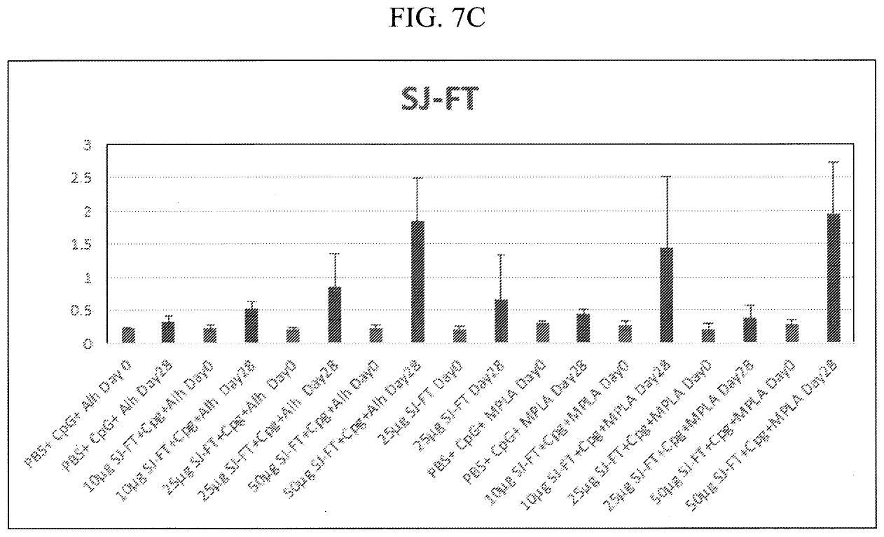

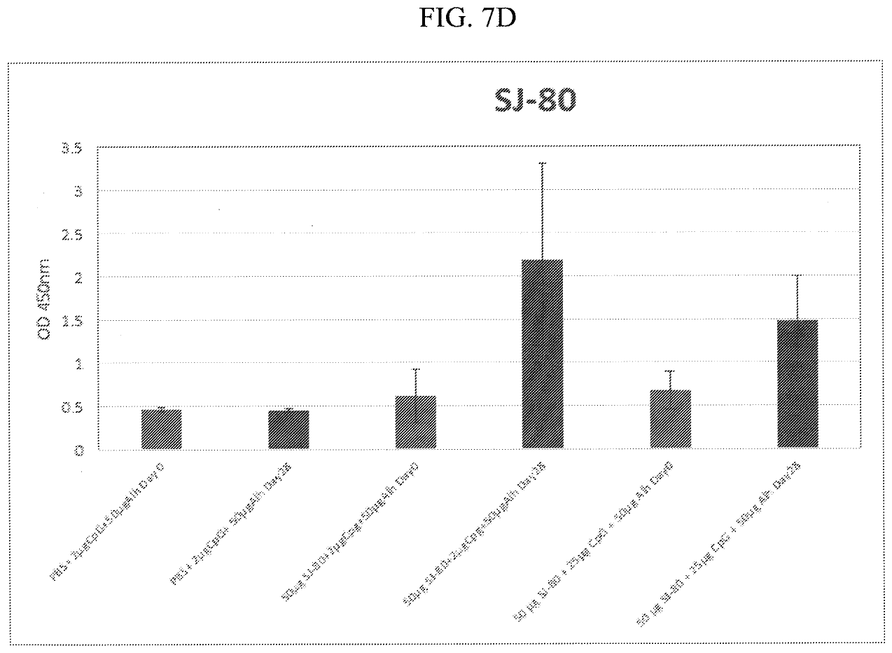

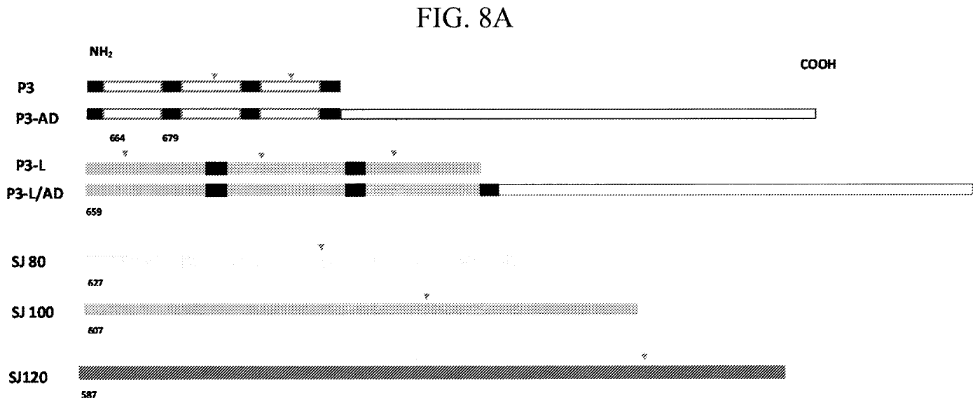

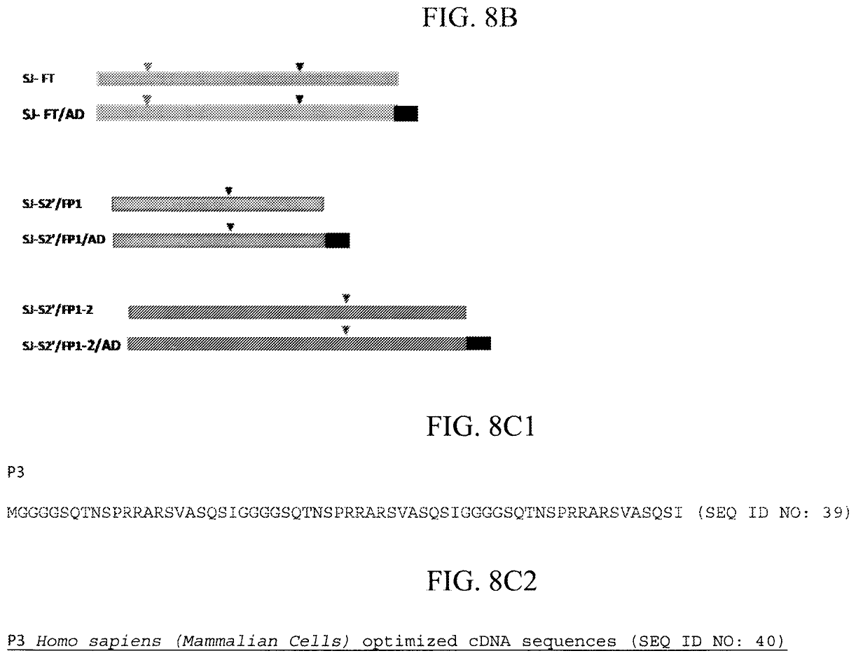

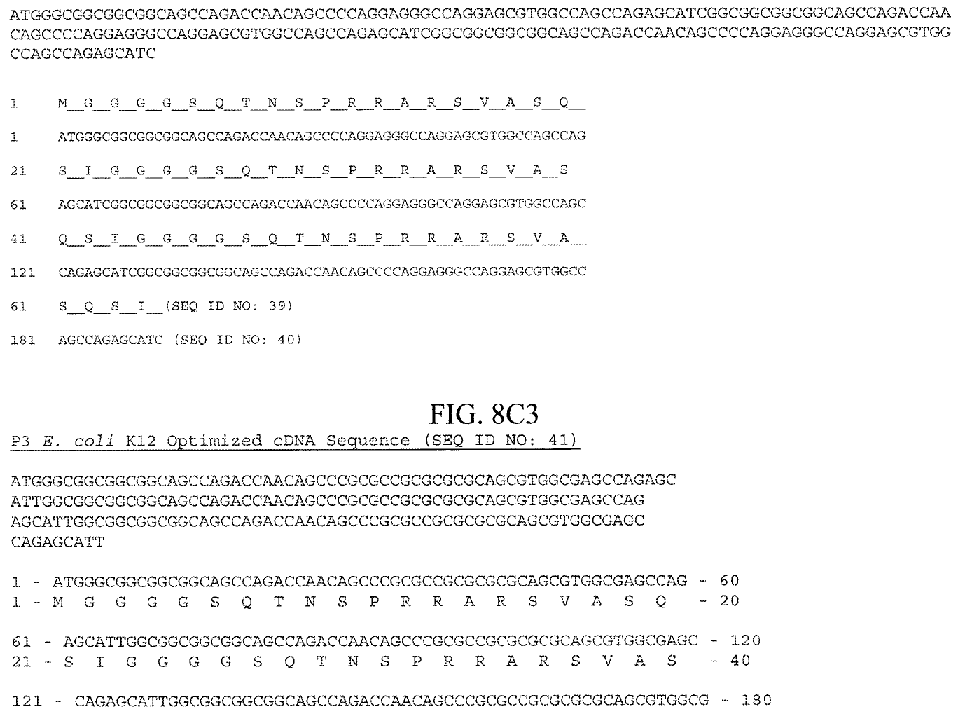

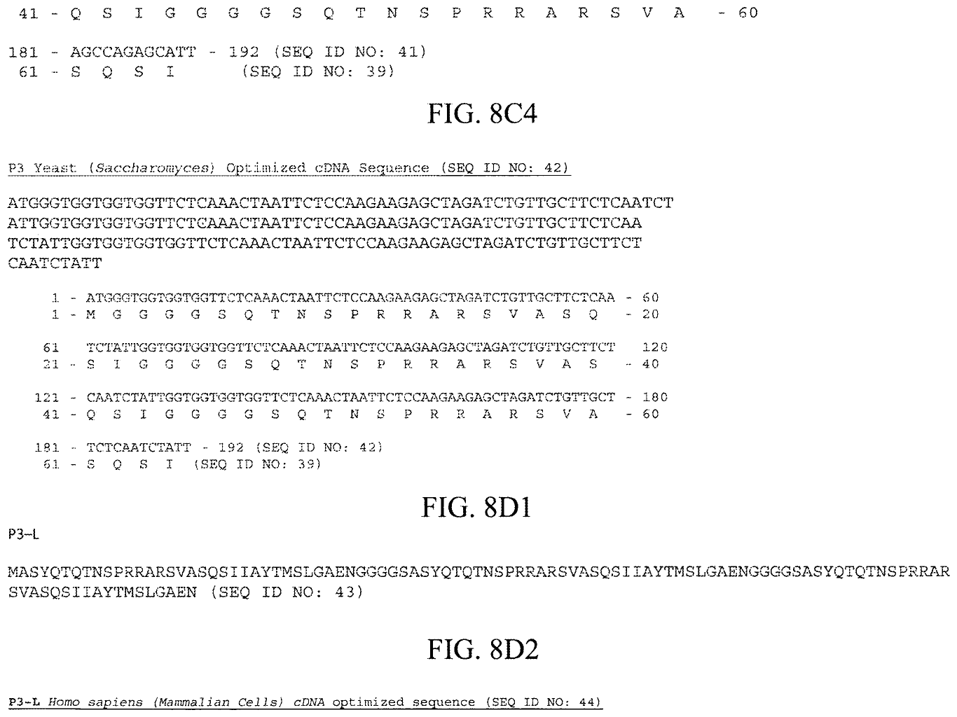

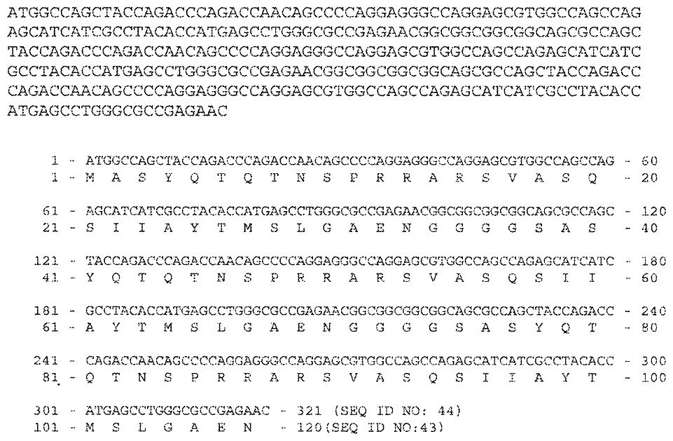

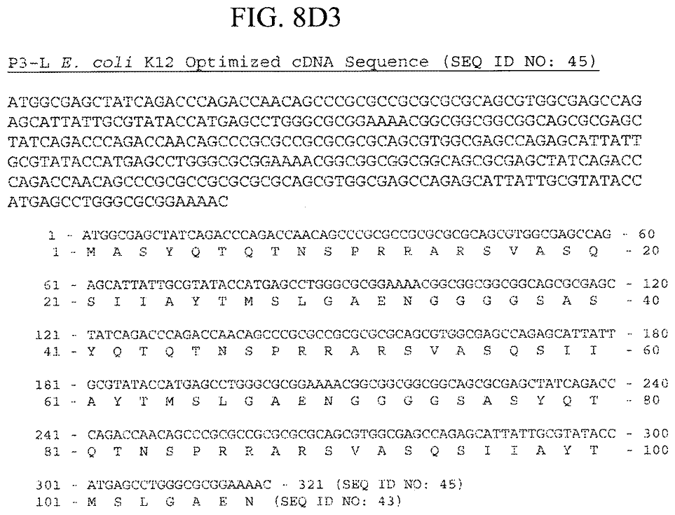

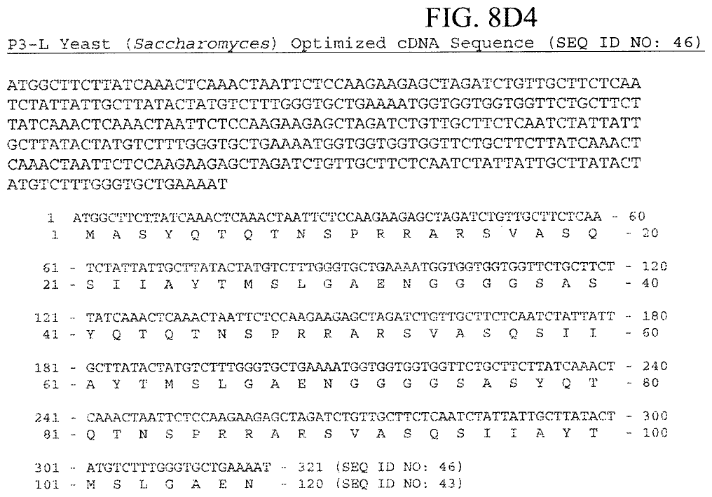

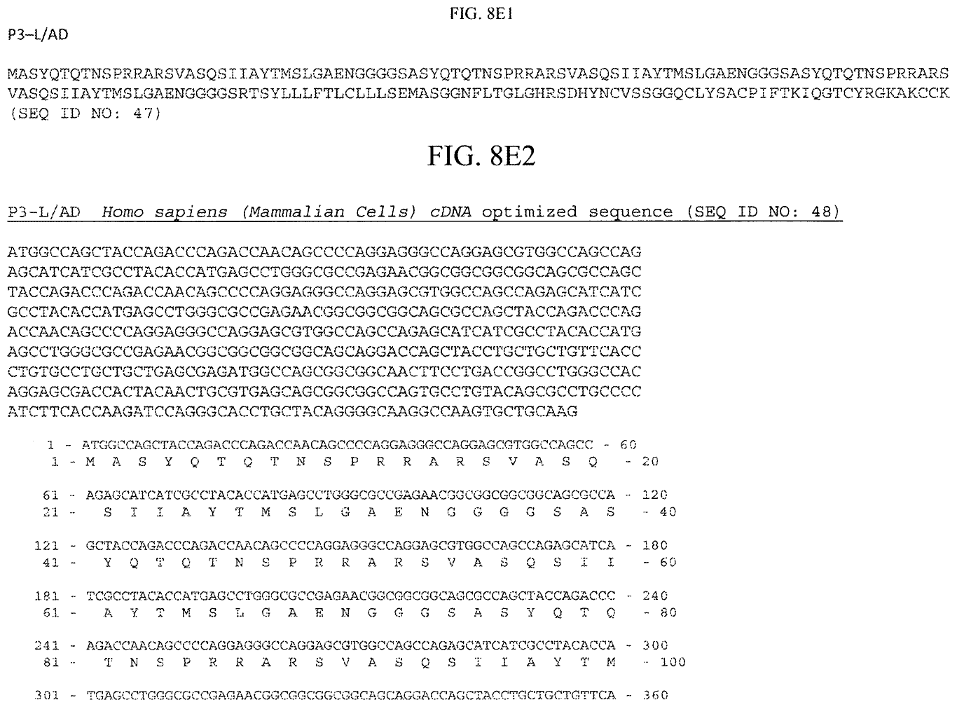

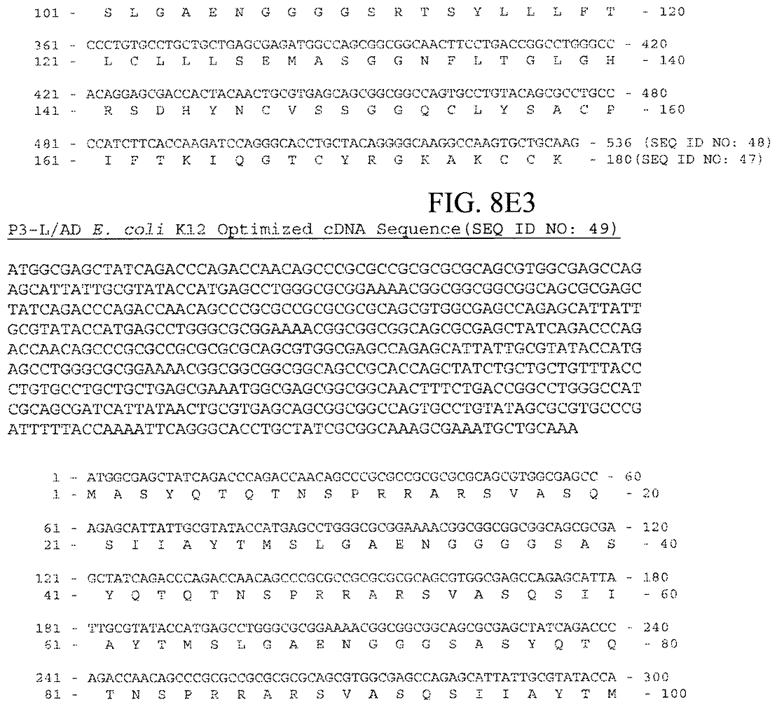

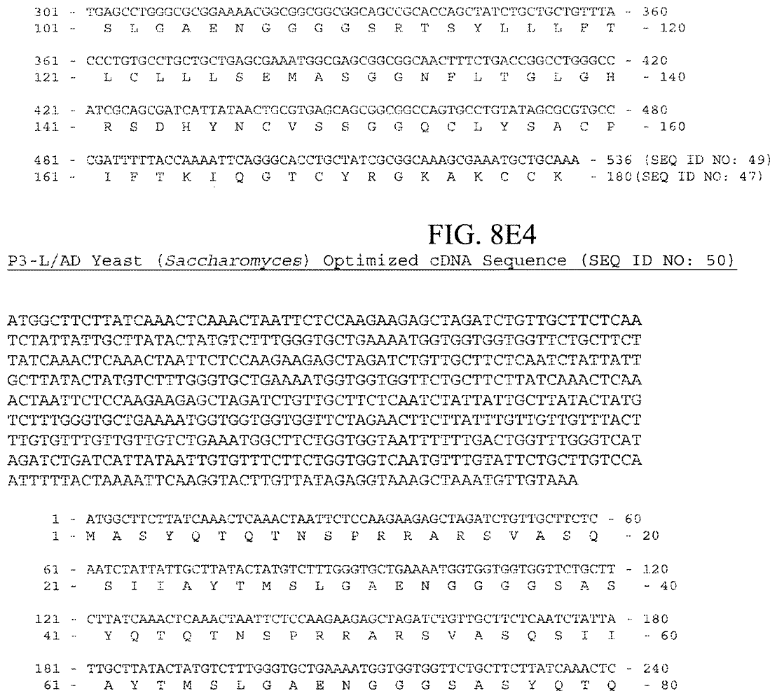

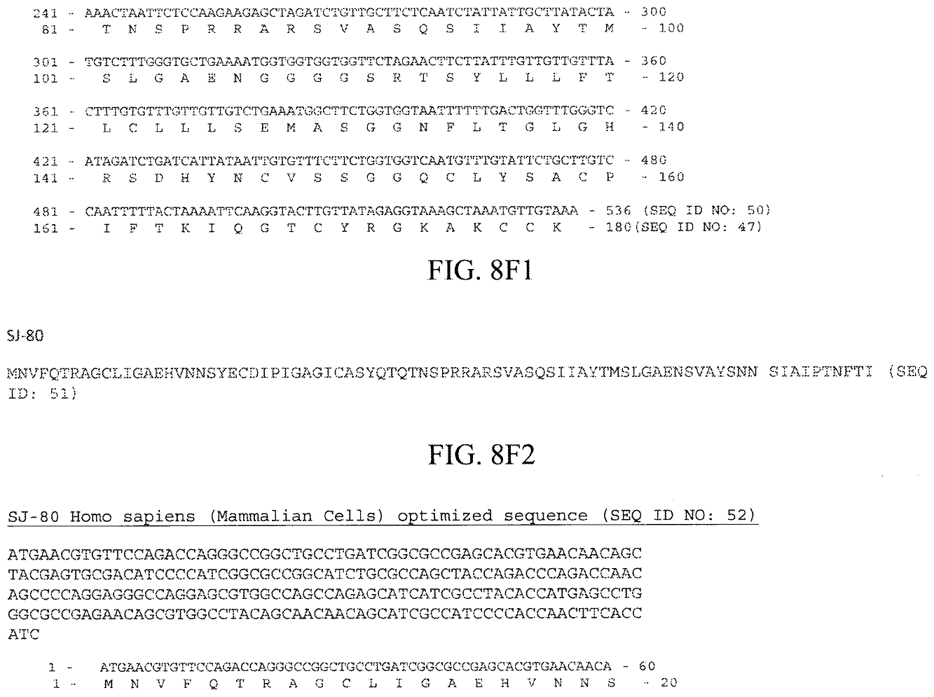

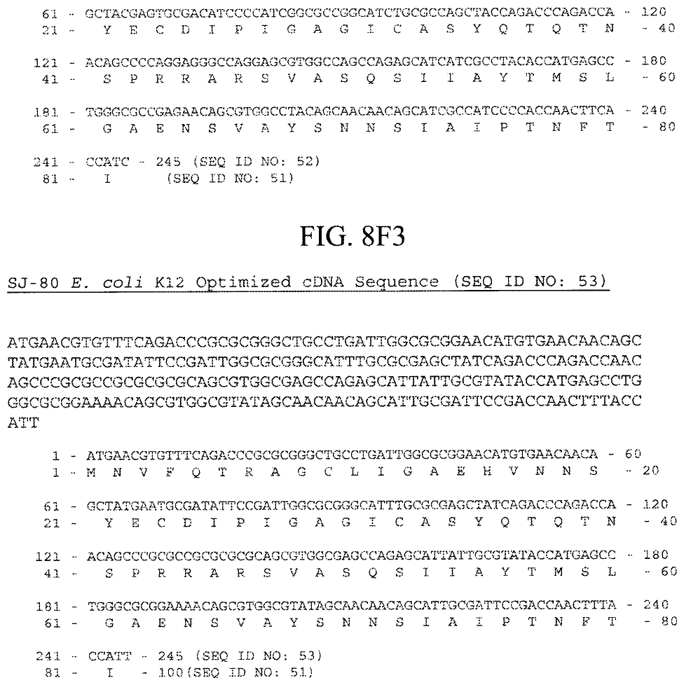

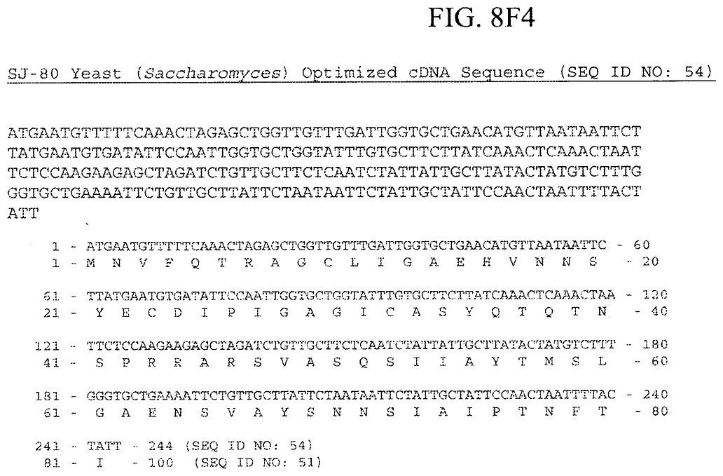

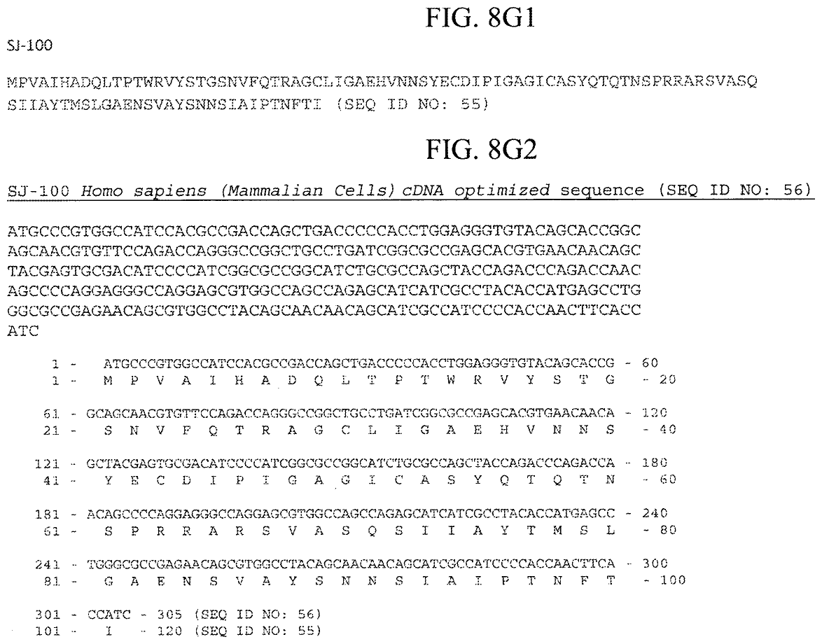

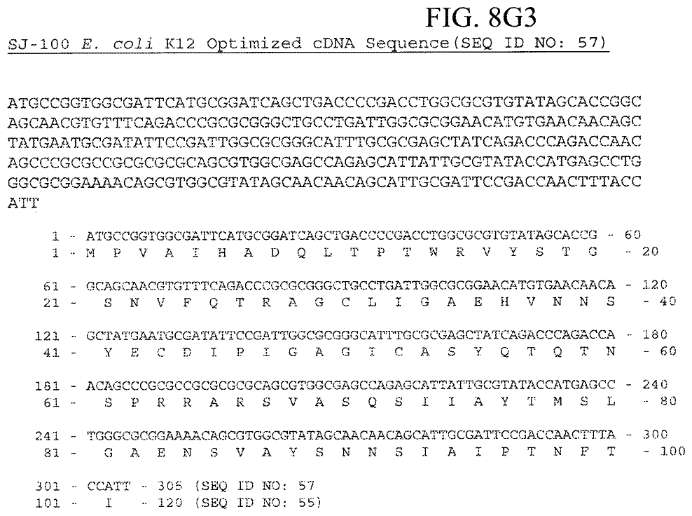

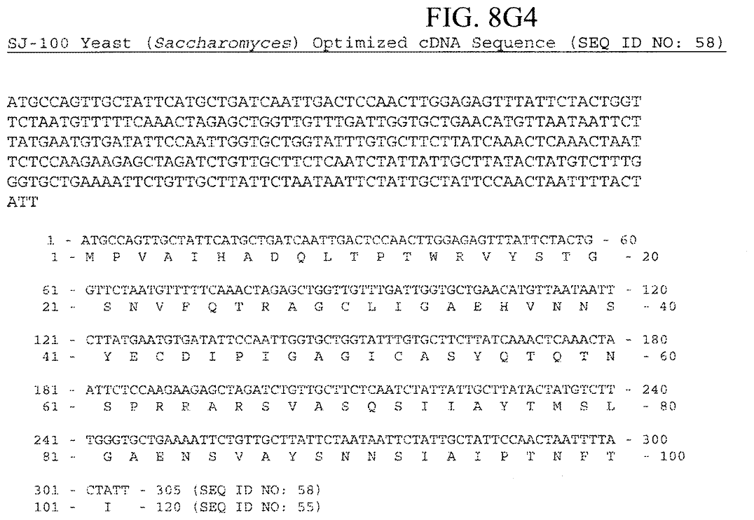

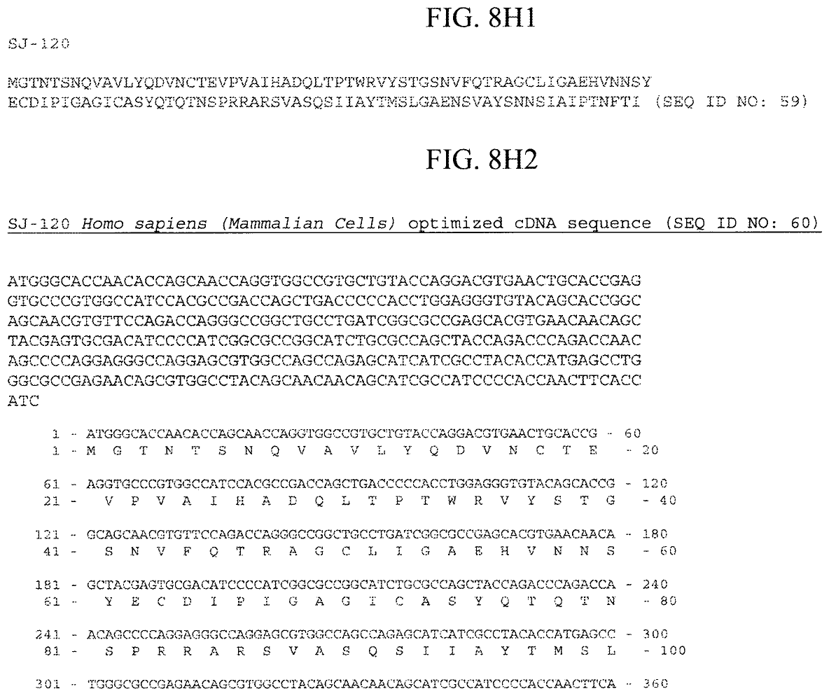

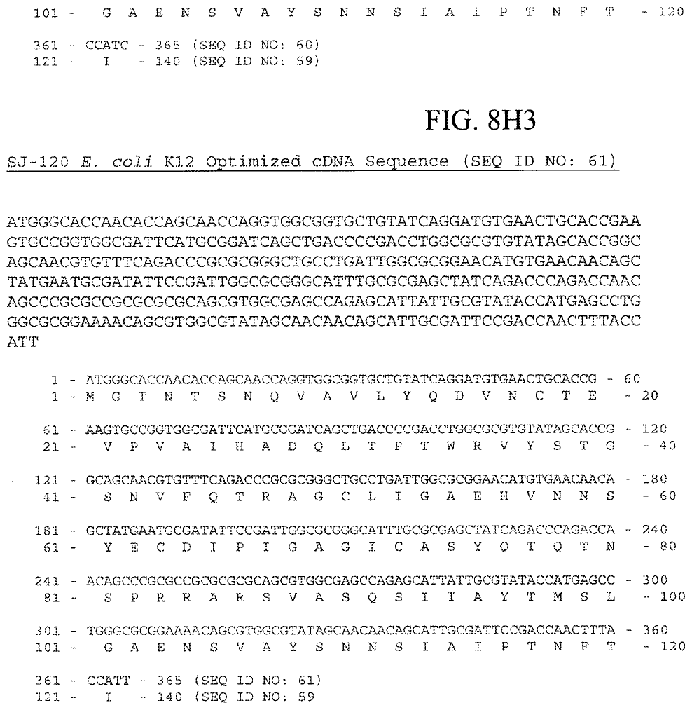

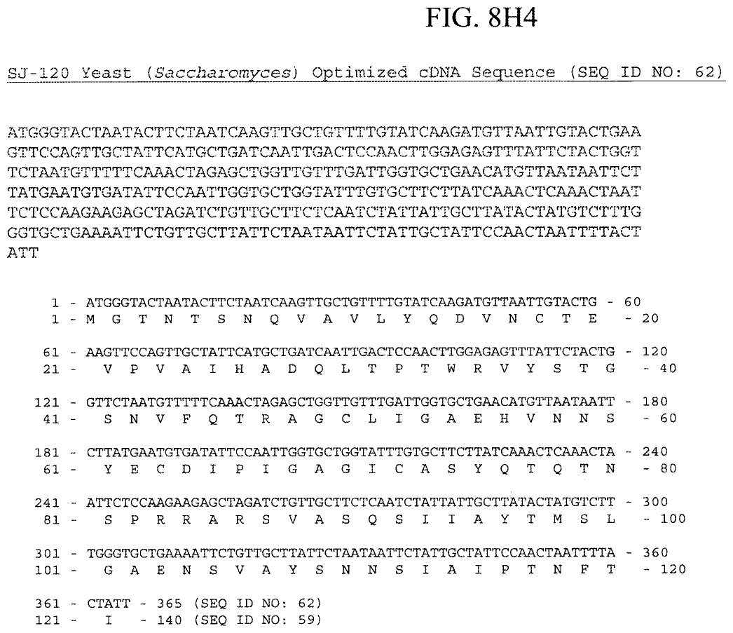

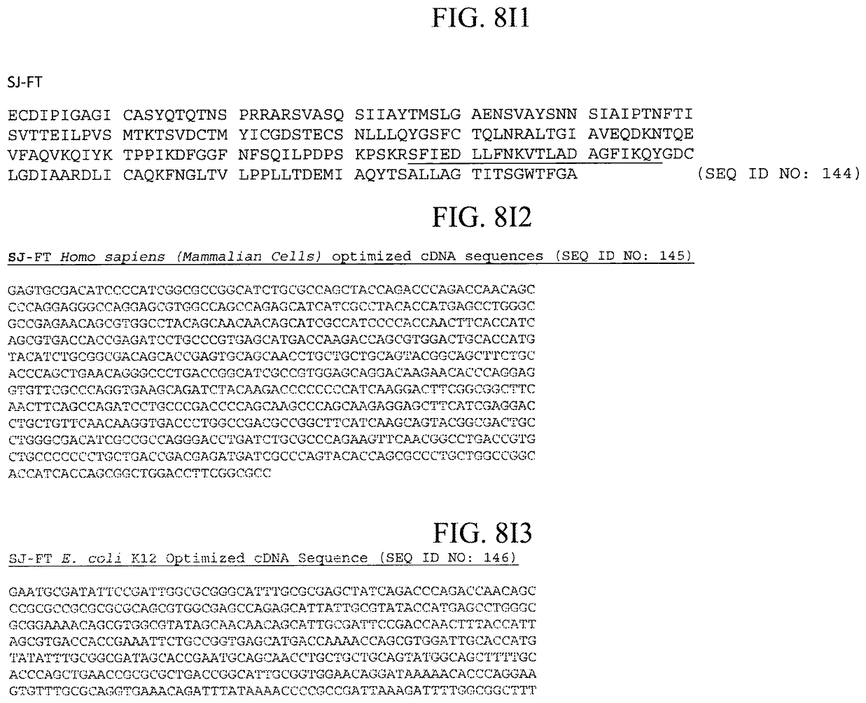

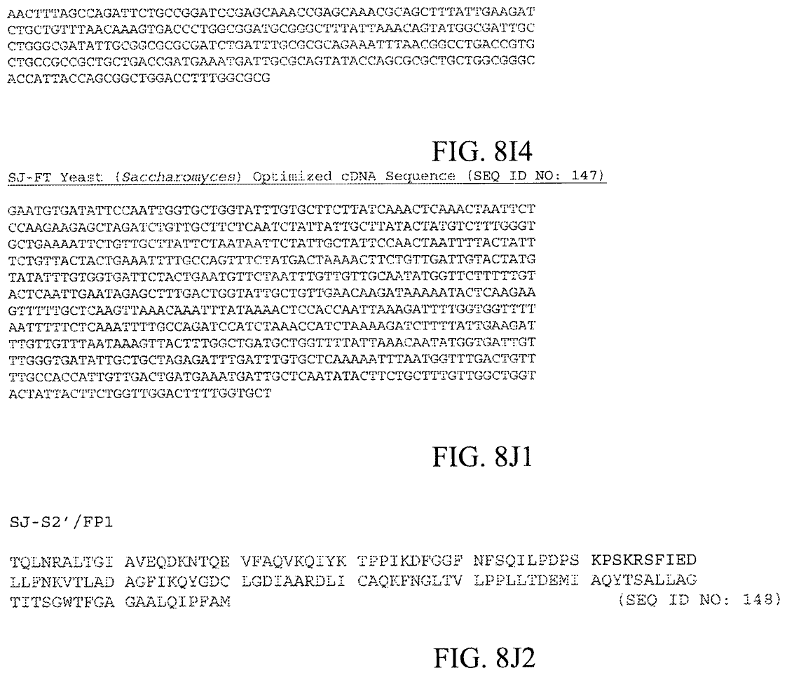

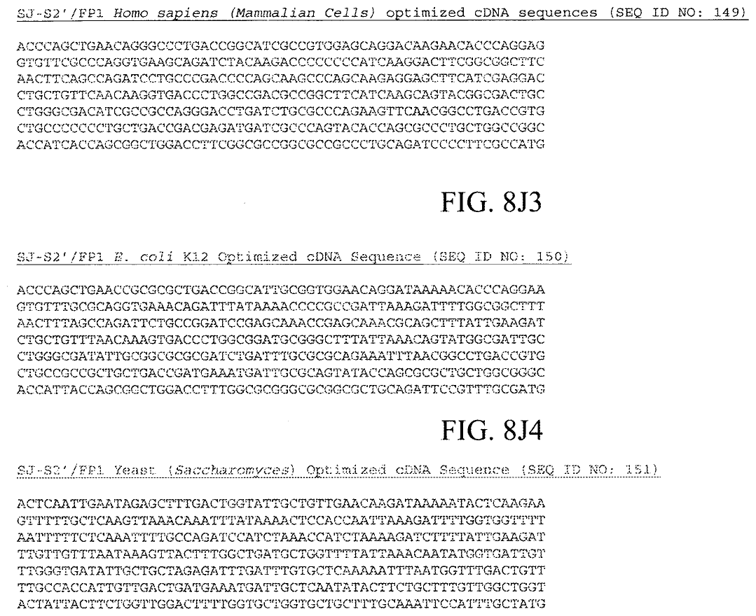

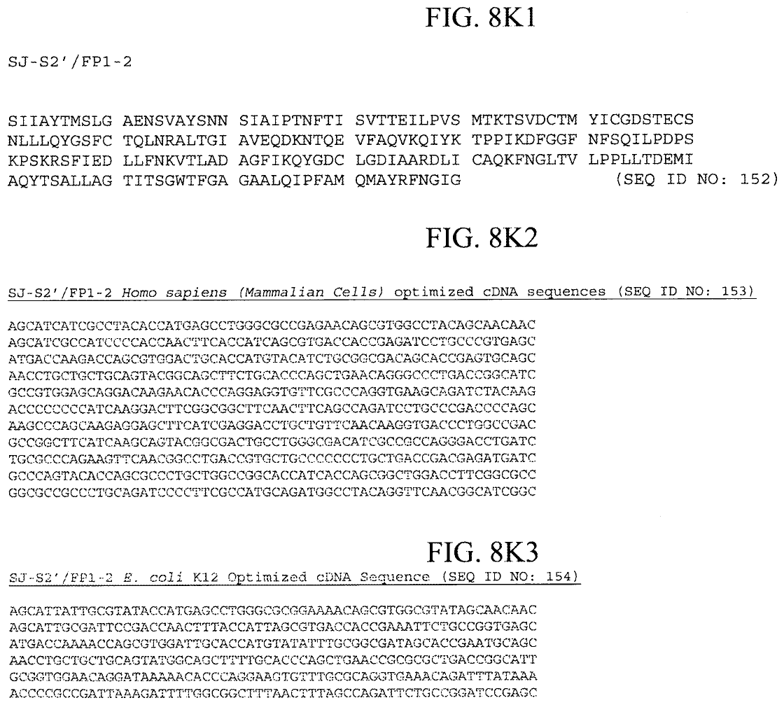

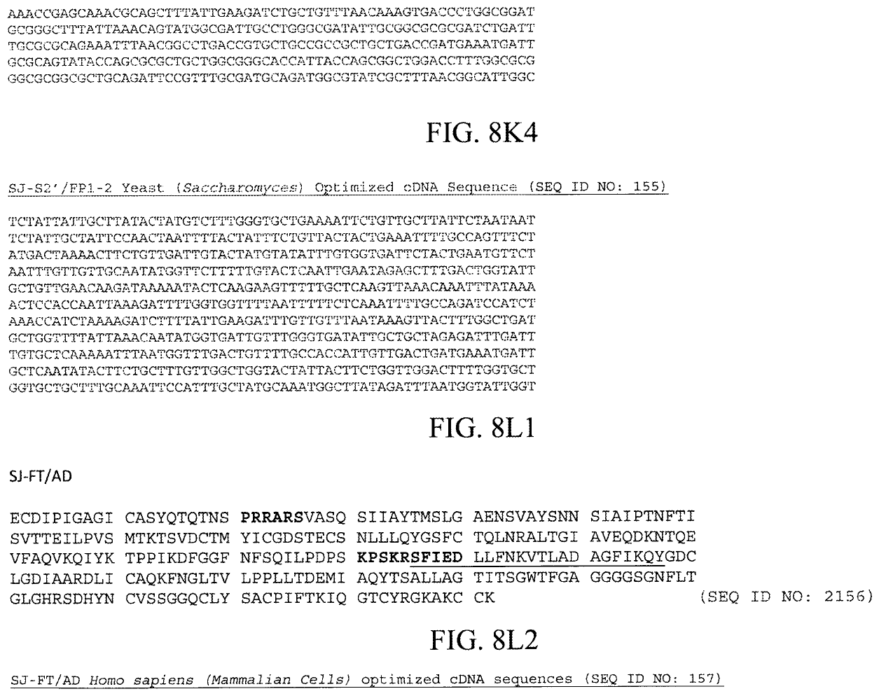

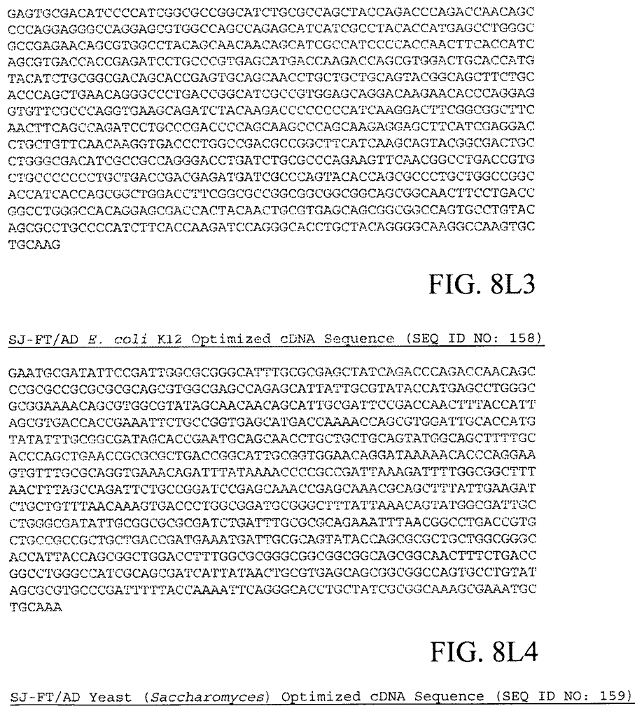

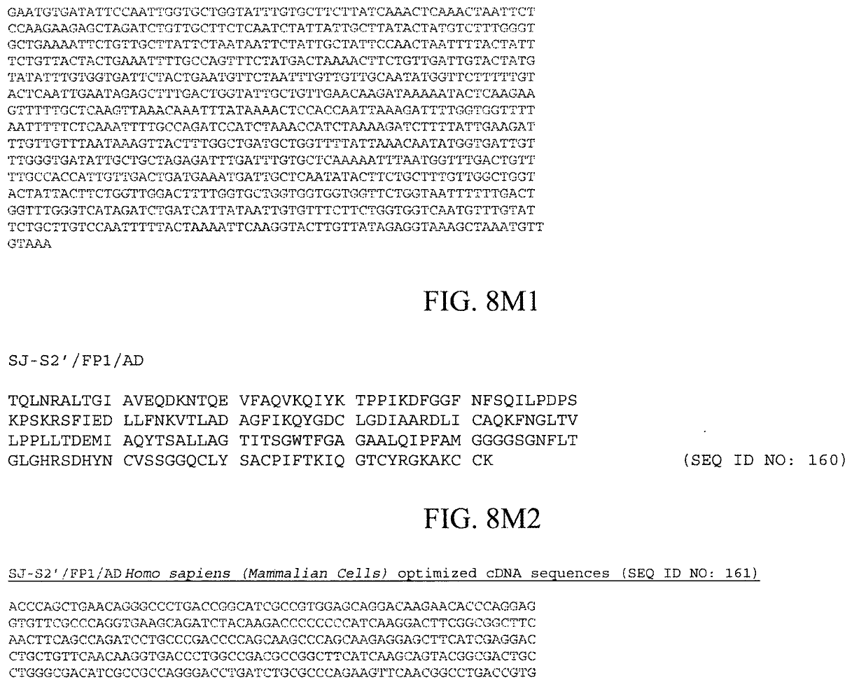

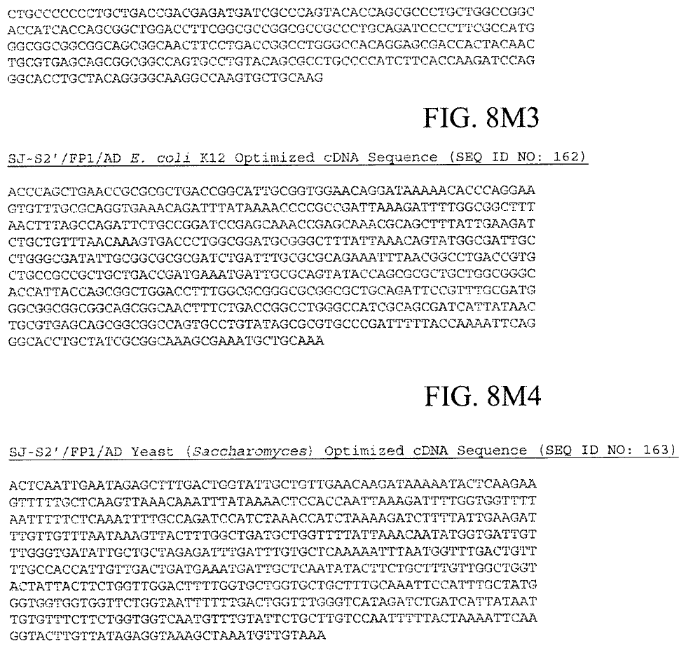

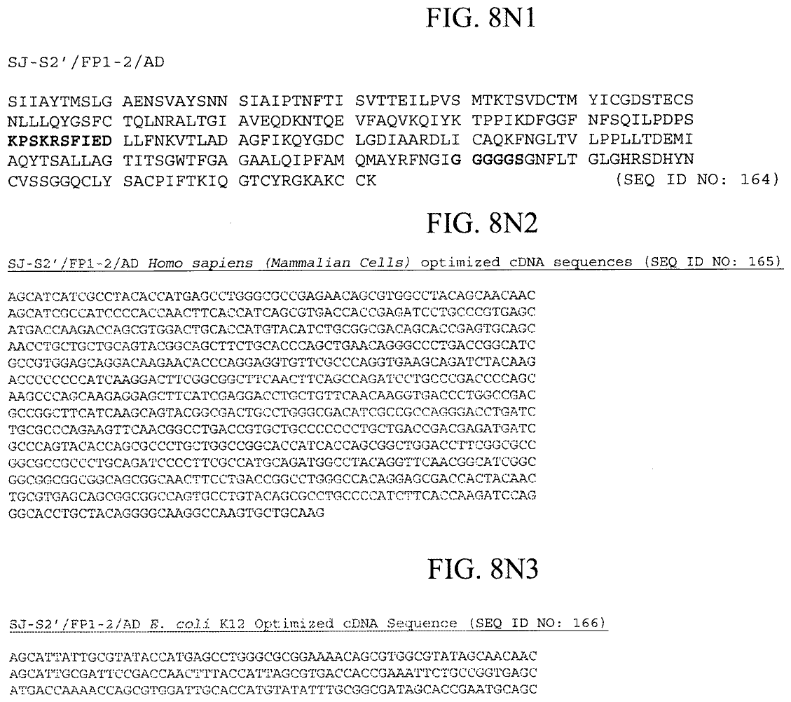

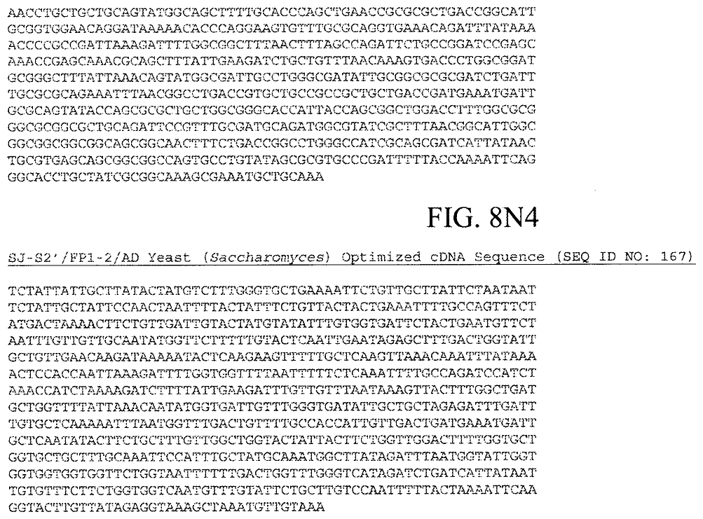

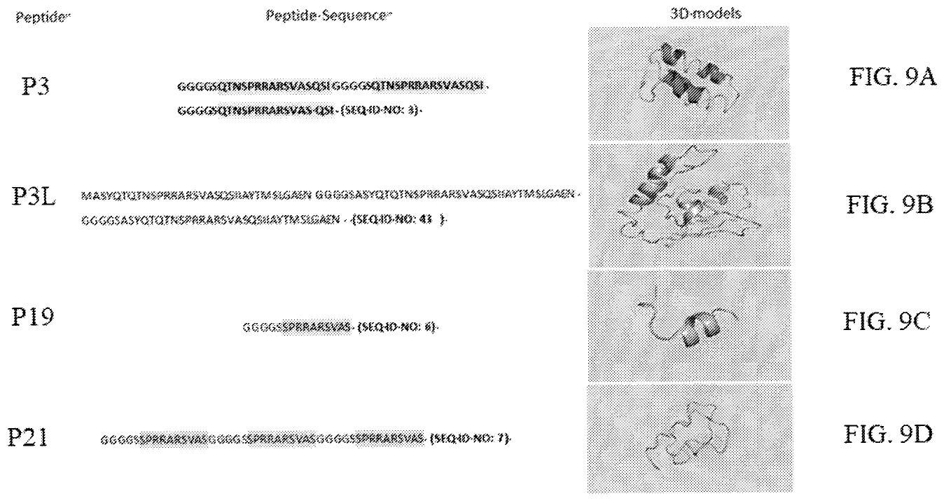

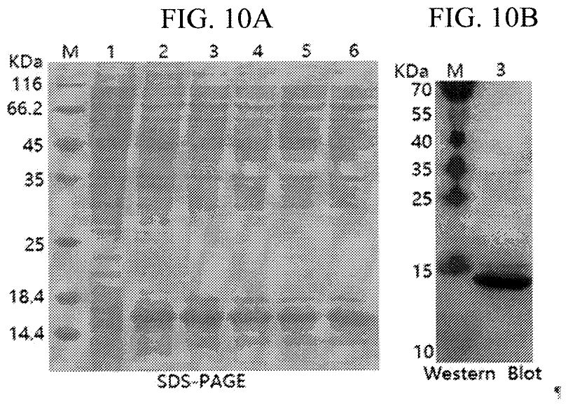

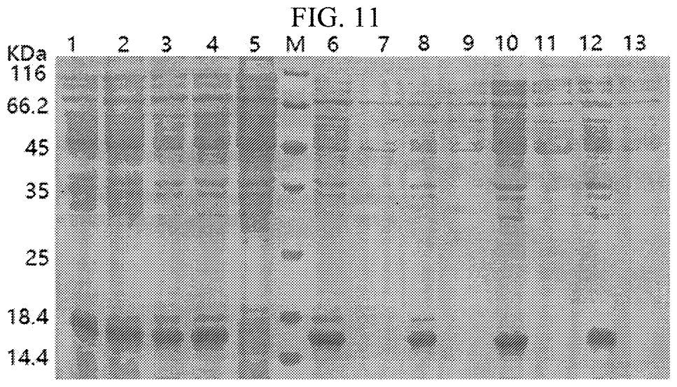

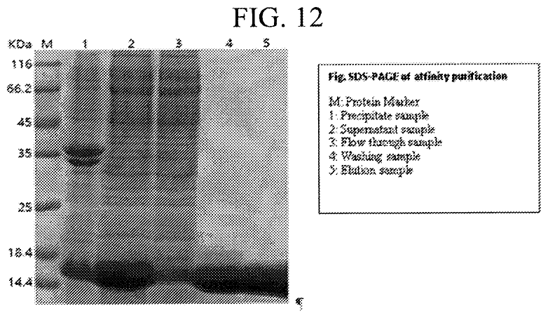

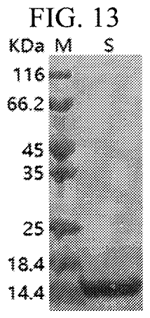

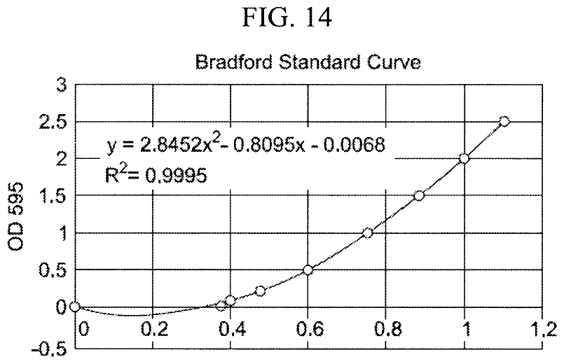

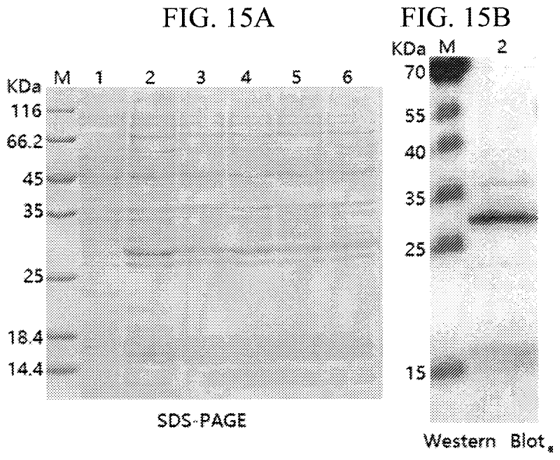

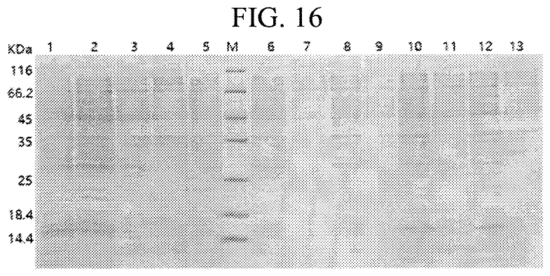

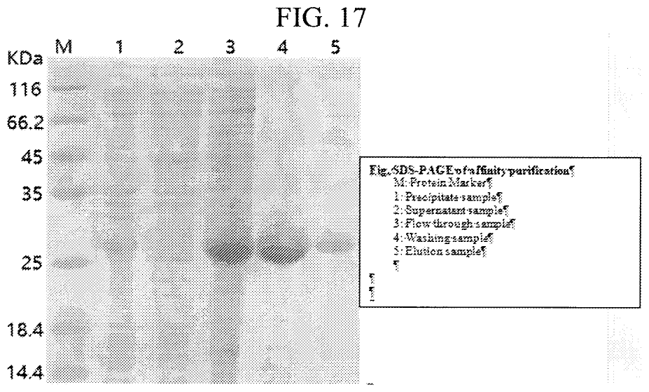

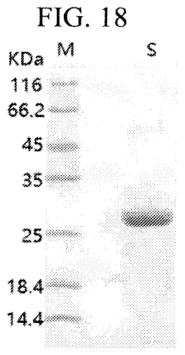

FIG. 4C-Data from the Local healthy control people collected during the pandemic,

FIG. 4D. Data obtained. with positive reference sera from the NIBSC.

FIGS. 5A-5D; Data of the ELISA assay using the sera from the cohort of PCR confirmed COVID19 patients with different designed antigens from the SARS-CoV-2 S protein, S1/S2 junction.

FIG. 5A. Polypeptide P3.

FIG. 5B. Polypeptide P4/TMP-Pol3.

FIG. Subunit P3L.

FIG 5D. Subunit SJ/FT.

FIG. 6. Dot plot chart produced using R and ggplot2 (v 3.3.3) library and Showing the distribution of “P3 OD” (Immune status) by “Disease status”. Bin width is 0.05. As In shown, there is a strong association between the immune (+) ‘Immunity status’ to antigenic determinants displayed in the P3 polypeptide and the asymptomatic and mild forms of the disease “Disease status”.

FIGS. 7A-7D: Mice antibody response [OD values] up to 100 days following immunization with polypeptides P3 (FIG. 7A), subunit P3L (FIG. 7B), Subunit SJ/FT (FIG. 7C) and Subunit SJ80 (FIG. 7D). Control mice received PBS. Three concentrations of the antigens (10, 25 and 50 ug) were tested along with CpG, Alhydrogel and MLA adjuvants. First bar in each set in FIG. 7A (Day 0), second bar (Day 28), third bar (Day 42) and fourth bar (Day 100).

FIGS. 8A and 8B; Multivalent Subunits Precision Vaccines Design.

FIG. 8A. The red arrowheads show the position of the PRRAR (SEQ ID NO: 107) motif. The black boxes show the GIS linker sequence. The white boxes show the 16 residues core sequence spanning the FCS motif PRRAR (SEQ ID NO: 107). The green boxes show the 32 residues core sequence spanning the PRRAR (SEQ ID NO: 107) motif. The Yellow boxes show the sequence of the human beta 1 Defensin attached to the COOH terminal of the vaccinating subunits to enhance antigenicity. This sequence can also be attached to the NH2 termini of the subunit's vaccine.

FIG. 8B. The S1/S2/S2′ sequence spanning the Furin and TMPRSS2 proteolytic sites is shown in cyan blue. The S1/S2′ Sequence Spanning the TMPRSS2 cut site and containing at least 4 antigenic epitopes is shown in gold. Red arrowheads=Furin cut site, Black arrowheads=TMPRSS2 cut site;

FIG. 9: Ab initio 3D modeling of designed peptides. Peptide P3 with the 3 repeats of the 16 residues core sequence (in Blue) and P3 L with the 3 repeats of the 32 residues core sequence (in yellow), and the P19 with the shorter 10 residue sequence tin green). The three sequences encompass the PRRAR (SEQ ID NO: 107) motif and display an Alpha Coil/Helix fold associated with epitope recognition by COVID19 patient's sera, The P21 peptide with the three repeats of the short P19 peptide sequence, showed a different Told that is not recottnized by the COVID19 patients' sera. 3D Models 1, 2, and 3 are useful to design antagonists of the SARS-CoV-2 S protein Furin cut site.

FIGS. 10A and 10B respectively show the stained SDS-PAGE gel and Western Blot of polypeptide antigen PL 3. M: marker proteins, Lane 1: Uninduced sample, Lands 2-6 induced samples.

FIG. 11 shows the results of optimization and solubility analysis for PL 3

FIG. 12 shows results of SDS-PAGE of the affinity purified materials associated with PL 3.

FIG. 13 FIG. 13 shows an SDS-PAGE of the purified PL 3 protein.

FIG. 14 shows a Bradford Standard Curve used for determining protein concentration.

FIGS. 15A and 15B respectively show the stained SDS-PAGE gel and Western Blot of polypeptide antigen SJ-FT. M: marker proteins, Lane 1: Uninduced sample, Lands 2-6 induced samples.

FIG. 16 shows SDS-PAGE results of optimization and solubility analysis for SJ-FT,

FIG. 17 shows results of SDS-PAGE of affinity purified materials associated with SJ-FT.

FIG. 18 shows the. SDS-PAGE result of the purified protein. M corresponds to the Protein Marker, S corresponds the target protein.

FIGS. 19A-30D respectively show the nucleic acid sequences coding for polypeptides P3, P3-L, P3-L-AD and subunits S1-80, SJ-100, SJ-120, SJ-FT, SJ-FT/AD, SJ-S2′/FP1, SJ-S2′/FP1/AD, SJ-S2′/FP1-2, SJ-S2′/FP1-2/AD followed by codon-modified nucleic acid sequences for expression in mammalian cells, Escherichia coli K12, and the yeast Saccharomyces Cerevisiae.

### DETAILED DESCRIPTION OF THE TECHNOLOGY

Unless specifically defined, all technical and scientific terms used herein have the same meaning as commonly understood by a skilled artisan in enzymology, biochemistry, virology, cellular biology, molecular biology, and the medical sciences. All methods and materials similar or equivalent to those described herein can be used in the practice or testing of the present invention with suitable methods and materials being described herein.

Indroduction. While medical therapy using, anti infections drugs such as antibiotic and antiviral agents is fundamental in the fight against infectious diseases, vaccines are instrumental in providing immunity and protection against such diseases that can develop into epidemics and sometime into pandemics. As for the COVID19 pandemic that is sweeping the globe since late 2019, no consensus on an effective medical therapy has emerged so far particularly for the treatment of the severe and deadly forms of the disease. Therefore, vaccination is on top of the options and unprecedented efforts are being deployed to develop efficient vaccines to prevent the disease and stop the pandemic from spreading. However, and despite an overall good efficiency, the currently existing vaccines have a number of pitfalls and ought not be the most appropriate to fight SARS-CoV-2. Tire ongoing efforts to develop more potent and safer vaccine are favoring the use of modern approaches combining the latest computational modeling and predictive tools (Crooke et at, 2020; Nanishi et al., 2020; Vashi et al., 2020) with experimental work while integrating the latest knowledge in immune response epidemiological, clinical and genetic patients-centered data.

The first vaccines developed to fight SARS-CoV-2 were developed using the latest technological advances such as RNA-mediated vaccination and/or recombinant viral shuttle delivery systems (Bos et al., 2020: Kremer, 2020: Polack 2020: Schlake et al., 2012; Sun et al., 2021; WHO 2021). However, they tall used for immunization a no precision targeted approach based on the administration of the whole S spike protein as antigen, They were also not based on a patient-centered approach. Indeed, the highly efficient mRNA-based PfizeriBioNtech vaccine, as an example, delivers to the immune system a mutated form of the antigenic full-length spike protein of SARS-CoV-2S virus spike protein in the prefusion form to reduce fusion to target cells membranes. Knowing that the proteolytic cleavage of the S protein is a major process of the virus infectivity, immunization with the mutated full length prefusion form of the S protein will generate antibodies to the protein most antigenic, inmumodominant epitopes but will not likely generate antibodies that would cause steric blockage of the native virus proteolytic sites; This may partly explain the infection of vaccinated patients with SARS-CoV-2 variants different from the vaccine one. Furthermore, it has been shown that inhibition of the endogenous fur-in proteolytic activity suppresses mulncycle replication of SAPS-CoV-2 in human airway epithelial cells (Bettes et 2020).

Identification qt. S1/S2 peptides. The inventors considered that a vaccination approach inspired from the effective protective immunity elicited by natural infection would be more effective, the inventors searched for antigenic determinants that are associated with a protective immunity and focused their efforts investigation on the junction of the S protein S1/S2/S′2 subunit and particularly on the sequence spanning the furin and TMPRSS2 cut sites. The inventors considered that an antibody response directed to this presumably exposed sequence that harbor important functional parts of the S protein might interfere with the proteolytic activation of SARS-COV-2 and thus would inhibit membrane fusion and limit the virus potential to enter the target cell.

The inventors initially identified a sequence of the S protein located at the 5I/S2 subunits junction and showed that it contains antigenic determinants that were strongly recognized by antibodies found in asymptomatic to mildly affected SARS-CoV-21PCR confirmed COVID19 patients.

The inventors found that there was a very high association between responses to this multivalent sequence and milder forms of the disease and rapid recovering from COVID19. This provides proof that the antigenic determinants identified by the inventors elicit protective immunity. This was again confirmed by immunizing mice with. these antigenic determinants which were found to elicit strong specific antibody responses against SARS-CoV-2. Their work also provided an explanation of the observation of this type of antibody response in the presumably healthy controls enrolled during the pie of the pandemic and the negative WHO reference samples from the NIBSC/UK, whom have most probably been infected sub clinically. In addition, the discovery using various immunoinformatic tools of antigenic epitopes in the defined sequence spanning the SlIS2 junction particularly the 14 peptides bearing highly probable B and T cell epitopes predicted to be generated by the proteasome/immunoproteasome of an antigen presenting cell provides a structural basis to the serological data obtained with the sera of recovered COVID19.

Interestingly, the antibodies generated by these antigenic determinants not only provided protective immunity but also persisted for more than 12 months, which is consistent with a report about the persistence of antibody response to SARS-CoV-2 for months (Wajnberg et al., 2020). Furthermore, this antibody response uncovered by the inventors does not cause antibody-dependent enhancement of the infection (Lee et al. 2020; Katthik et al. 2020) or cross react with other furin cut motifs displayed by the endogenous mammalian proteins listed in hypertext transfer protocol://www.nuolan.net/imotif.html (Tian el al. 2011, Tian et al. 2012) indeed the patients' who developed this type, of immunity experienced rapid and full recovery and no reinfection was recorded among them to date as per our record. This .is highly M favor of the vaccinating potential of the antigenic determinants we uncovered. In addition, in the sequence surrounding the TMPRSS2, four antigenic peptides were identified and one of these peptides was found to encompass the TMPRSS2 cut site.

The inventors subsequently developed six other sequences of different lengths (SJ-80, SJ100 and SJ-120, SJ/FT, SJ-S′2/FP1 and SJ-S′2/FP1-2) spanning SARS-CoV-2 S protein S1/S2/S2′ subunits junction and encompassing the furin and TMPRSS2 cut site motifs and the other antigenic peptides.

These multivalent sequences were selected based on their antigenic, multiple MITC interactions, no allergenic, and physiochemical properties that makes them good candidates for a targeted precision anti SARS-CoV-2 vaccine. In contrast, the prior vaccines, the inventors pursued a vaccine that: was versatile and can be adapted to various vaccination technologies and which can be delivered through multiple routes particularly as recombinant subunit vaccines or using recombinant viral shuttle or as mRNA vaccines depending on the ease and cost of production, the stor age conditions, the robustness of material and the safety profile. They also pursued a safer vaccine that does not require immunization with an. entire S protein which is a biologically active protein that can interfere with physiological processes involving the ACE2 receptor and cause unwanted side effects such as platelet aggregation. thrombosis and exacerbated inflammation (Angeli, et al. 2021). In addition, immunization using full-length protein-based approach can lead to ADE as reported in MERS-CoV (Prompetchara et al. 2020).

To carry out the patient-centered-study upon which the inventors defined antigenic determinants of the SARS-CoV-2 virus that are associated with protective immunity, the inventors developed an indirect ELISA assay, This assay was developed by testing several antigens and selecting a multi-epitope peptide for the study of the immune response in a cohort of around 500 people infected by. SARS-CoV-2 assessed clinically and confirmed by positive PCR testing of the presence of the virus specific RNA. Since immunity to SARS-CoV-2 is yet not fully understood and that tracking; protective immunity is an issue in COVID 19, the development of this assay provides a unique serological tool to study and track natural or post vaccination protective immunity to SAR-CoV-2 infection.

The ongoing corona virus disease (COVID19) pandemic caused by the emergence of the new coronavirus SARS-COV-2, has triggered a critical and urgent need for the development of highly efficient and sate vaccines as well as diagnostic and therapeutic tools. To address this need, the present invention provides antigenic determinants located in a region of the virus spike S protein involved in SAR-CoV-2 high infectivity. These antigenic determinants were identified using rational and computational approaches and were validated by experimentations and clinical data.

The furin cut site (FCS), found exclusively in the S protein of SARS-CoV-2 clade, lays in this region. The present invention includes the identification of a series of 14 small peptides spanning the S protein S1/S2 subunits junction and containing B and T cells epitopes. Five of these peptides contain or overlap with the FCS motif (PRARR). A series of peptides with this motif as a core sequence were prepared and their 3D models generated in silico.

Based on the work underlying the present invention, we developed a series of multivalent subunits derived from the S protein S1/S2/S2 junction sequence that span the sequences of the peptides and were found to elicit a protective immune response. Computational analysis tools showed that these subunits were structurally stable, antigenic and non-allergenic, thus suitable for human precision vaccination. Hence, the data generated from this patients-centered approach study allowed for the development of an ELISA assay for the tracking of SARS-CoV-2 protective immunity.

Also provided in the present, invention are vaccine subunits that mimic the natural protective immunity elicited by targeting the structural region of the virus associated with. infective power. The developed peptides and protein subunits were designed to trigger effective immunity against all SARS-CoV-2 genetic variants. The developed structural 3D models are useful for the design of precision drugs that interfere with SARS-CoV-2 infectivity.

The present invention also provides an indirect ELISA. assay for assessing antibody response. By using this indirect ELISA assay, the present invention shows that. the antibody response, to one of these peptides (P3), is highly associated to the asymptomatic and mild forms of the COVID19 in a cohort of 492 SARS-CoV-2 PCR-positive COVID19 patients [p<0.0001]. Thus, the antigenic determinants present in the P3 sequence elicit protective immunity. Further, immunization of BALBc mice with two of the engineered polypeptides (P3 and P4/TMP-Pol3), using Freund complete adjuvant or-vaccine grade CpG adjuvant, elicited strong humor al immune response.

Some non-limited embodiments of the technology of the invention include the following.

1) A first set of peptides/polypeptides based on P1, P2, P3, P4, P5, P9, P19, P22, P23, P24 of various sizes and derived from the S protein S1/S2 subunits junction sequence which are exclusive to SARS-CoV-2, These peptides/polypeptides ere developed using a mix of rational and computational design that predicts the proteasome/immunoproteasome generated peptides putatively binding to MHC antigen presenting molecules and/or potential B cell epitopes.

2) SARS-CoV-2 vaccines that incorpor ate as antigenic peptides one or more of the peptides/polypeptides based on P1, P2, P3, P4, P5, P9, P19, P22, P23, P24, P1/TMP, P2/TMP, P3/TMP, P4/TMP

3) An indirect ELISA protocol that detects specific antibodies generated exclusively ti against SARS-CoV-2-specific antigens (e.g., peptides based on P3) in a COVID patient's sera. This assay is useful in tracking anti SARS-CoV-2 protective immunity.

4) An ELISA-based method to evaluate COVID19 recovered patients who had the asymptomatic or mild form of the disease, which shows that these subjects developed high levels of specific antibodies to at least one of these peptides (e.g., peptides based on P3) and a to protective immunity [Very high association between anti P3 antibody response and disease asymptomatic/mild forms].

5) A second set of protein subunits and their corresponding cDNAs [e.g. P3-AD, P3-L, P3-L/AD, SJ-80, SJ-80/AD, SJ-100, SJ-120, SJ-120/AD] derived from the SARS-CoV-2 S protein S1/S2 subunits junction sequence, designed by computational prediction of the proteasome/immunoproteasome generated peptides putatively binding to MHC antigen presenting molecules and/or representing potential B cell epitopes.

6) SARS-CoV-2 vaccines that incorpor ate as antigenic peptides one or more of the peptides/poly-peptides based on P3-AD, P3-L, P3-L/AD, SJ-80/AD, SJ-100, SJ-100/AD, SJ-120, SJ-120/AD, SJ/FT, SJ/FT/AD, SJ-S′2/FP/AD, SJ-S′2/FP1-2 and SJ-S′2/FP1-2/D. The vaccine(s) can be used regardless of the virus genetic variations.

Also envisioned in the present invention is that the vaccines may comprise the antigenic peptides, DNA (including vectors) encoding the antigenic peptides, and/or mRNA encoding the antigenic peptides,

Other embodiments of this technology but are not limited to:

A polypeptide antigen comprising a SARS-CoV-2 S protein S1/S2 cut site or a S1/S′2 cut site, such as the P3 peptide (SEQ ID NO: 3), a variant thereof, or a chemically modified form thereof. This polypeptide antigen may comprise or further comprise a furin cut site (FCS) or a Transmembrane Protease Serine 2 (TMPRSS2) cut site.

The polypeptide antigen disclosed herein may comprise a peptide (or peptide sequence) selected from the group consisting of SEQ. ID NOs: 1-39, 43, 47, 51, 55, 59, 64-74, 108, 110-120, 144, 148, 152, 156, 160, and 164; or a chemically modified form thereof; from the group consisting of SEQ ID NOs: 1-30, 64-74, and 110-120; from the group consisting of SEQ ID NOs: 1-30, 64-74, and 110-120; from the group consisting of SEQ f NOs: 1-15, from the group consisting of SEQ ID NOs; 1-1(1 and 110-120; from the group consisting of SEQ ID NOs: 39, 43, 47, 51, 55, 59, 144, 148, 152, 156, 160, and 164; from the group consisting of SEQ ID NOs: 1-7, 9-12, 14-15, 18, 31-38 and 108; from the group consisting of SEQ 11 NO; 8 and SEQ ID NO: 13, These peptide/polypeptides contain epitopes located at or around the S1/S2/S′2 junction, This junction contains two -proteolytic cleavage sites, namely, the furin and TMPRSS2 sites. Without Proteolytic cleavage the virus can hardly gain entry to the host cells and is much less infective. The epitopes present in these peptides/polypetides are in the vicinity or overlap with the proteolytic cut sites, These peptides are immunogenic and have been shown to elicit protective immunity.

The polypeptide antigen disclosed herein may be a wild-type peptide that comprises in 6, 7, 8, 9, 10, 11, 12, 13 14, 15, 16, 17, 18, 19 to 20 or more contiguous amino acid residues of a SARS-CoV-2 antigen.

The polypeptide antigen disclosed herein may be a fusion or chimeric peptide or polypeptide or peptide conjugate comprising residues of SARS-CoV-2 as well as exogenous residues such as residues of a peptide carrier, an immunogen, or a peptide tag. The polypeptide antigen may comprise one or More contiguous or non-contiguous sequences of a SARS-CoV-2 S protein or other SARS-CoV-2 antigen or immunogen.

The polypeptide antigen disclosed herein may comprise one or more chemically modified amino acid residues, such as a N-terminal biotin group or a histidine tag, or other exogenous chemical group or chemical modification,

Another aspect of this technology is directed to a composition comprising a polypeptide antigen as disclosed herein, or a chemically modified form thereof, and a carrier, excipient, and/or adjuvant. In some embodiments, a composition may comprise two, three or more SARS-CoV-2 peptide sequences. The composition may induce humor al or cellular immunity against SARS-COV-2 when administered to a subject.

Another aspect of this technology is directed to a method for preventing or reducing the severity of an infection by SARS-CoV-2 comprising administering the polypeptide antigen disclosed herein, or a chemically modified form thereof to a subject in need thereof.

Another feature of this technology is directed to a method for detecting antibodies to SARS-CoV-2 in a biological sample comprising contacting the sample with at least one peptide, chimeric peptide, or peptide conjugate or a chemically modified form thereof as disclosed herein.

Another aspect of this technology is directed to a nucleic acid encoding at least one peptide, a chimeric peptide, or a peptide conjugate as disclosed herein. in some embodiments, the nucleic acid will comprise a wild-type polynucleotide sequence in other embodiments, it may comprise a codon-modified polynucleotide sequence. In some embodiments, the nucleic acid sequence may comprise one or more nucleotide analogs. Such a nucleic acid may be formulated as a composition comprising the nucleic acid as disclosed above and a pharmaceutically acceptable carrier, excipient and/or adjuvant.

An aspect of the invention is also directed to a method if preventing or reducing the severity of an infection by SARS-CoV-2 comprising administering to a subject in need thereof a nucleic acid encoding the polypeptide antigen or peptide product disclosed herein.

Technical Definitions

Analogs or variants of the polynucleotides or polypeptides disclosed herein, such as those encoding or describing SARS-CoV-2 peptides and polypeptides, may have different degrees of sequence identity or similarity to said polynucleotides or polypeptides. BLASTN may be used to identify a polynucleotide sequence having at least 50%, 55%, 60%, 65%, 70%, 75%, 80%, 85%, 37.5%, 90%, 92.5%, 95%, 97.5%, 98%, 99% or <100% sequence identity to a reference polynucleotide such as a polynucleotide comprising SARS-CoV-2 genomic, subgenomic, or antigen-coding sequences. A representative BLASTN setting modified to find highly similar sequences uses an Expect Threshold of 10 and a Wordsize of 28, max matches in query range of 0, match/mismatch scores of 1/−2, and linear gap cost. Low complexity regions may be filtered or masked. Default settings of a Standard Nucleotide BLAST are described by and incorpor ated by reference to hypertext transfer protocol secure://blast.ncbi.nlm.nih.gov/Blast.cgi?PROGRAM=blastn&PAGE_TYPE=BlastSearch&LINK_LOC=blasthome (last accessed Nov. 14, 2022).

BLASTP can be used to identify an amino acid sequence having at: least 50%, 55%, 60%, 65, 70%, 75%, 80%, 85%, 87.5%, 90%, 92.5%, 97.5%, 98%, 99% or <100% sequence identity, or similarity to a reference amino acid, such as a SARs-CoV-2 amino acid sequence, using a similarity matrix such as BLOSUM45, BLOSUM62 or BLOSUM80 where BLOSUM45 can be used for closely related sequences BLOSUM62 for midrange sequences, and BLOSUM80 for more distantly related sequences. Unless otherwise indicated. a similarity score will be based on use of BLOSUM62. When BLASTP is used, the percent similarity is based on the BLASTP positives score and the percent sequence identity is based ti on the BLASTP identities score. BIASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity or similarity to the amino acid sequences disclosed herein ate contemplated and encompassed by this disclosure. A representative BLASTP setting that uses an Expect Threshold of 10, a Word Size of 3, BLOSUM 62 as a matrix, and Gap Penalty of 11 (Existence) and 1 (Extension) and a conditional compositional score matrix adjustment Other default settings for BLASTP are described by and incorpor ated by reference to the disclosure available at: hypertext transfer protocol secure://blast,ncbi.nlm.nih.gov./Blast.cgi?PROGRAM=blastp&PAGE_TYPE =BlastSearch&LINK_LOC=blasthome (last accessed Nov. 14, 2022),

Analogs or variants of a polynucleotide may include those with 1, 2, 3, 4, 5, 6, 7, 8, 9, 10, 20, 30 or more deletions, substitutions, or insertions of nucleotides into a polynucleotide such as those disclosed herein.

Analogs or variants of peptides or polypeptides include those with 1, 2, 3, 4, 5, 6, 7, 8, 9, 10, 20, 30 or more deletions, substitutions, or insertions of amino acid residues into a polypeptide, such as those disclosed herein, Such analogs may be based on one or more peptides polypeptides encoded by SARS-CoV-2, such as S1 or S2 sequences.

“Fragment” or “functional fragment” as used herein refers to shorter or truncated segments of a longer nucleic acid or polypeptide sequence, preferably which retain at least one function of the whole nucleic acid or polypeptide, such as an epitope. Functional immunogenic fragments are thus suitable for vaccination purposes. Generally, a fragment of a nucleic acid encoding a SARS-CoV-2 antigen will encode at least one epitope thereof and a fragment of a SARS-CoV-2 antigen will comprise at least one epitope of the antigen. A fragment may retain one or more functions of a longer peptide or polypeptide, such as immunogenicity or antigenicity. Examples of polypeptide fragments include fragments of longer SARs-CoV-2 polypeptides having at least 6, 7, 8, 9, 10, 11, 12, 13, 14, 15, 16, 17, 18, 19, 20, 30, 40, 50 or more contiguous amino acid residues. Examples of polynucleotide fragments include those encoding the peptide or polypeptide fragments described above.

The term “AD” as used herein refers to the adjuvanted form. Adjuvanted forms and means of making the same are well-understood by the person skilled in the art. As an example of an ad wanted from reference is made to an adjuvanted form having the human β1 defensin sequence added to the C-terminus of these multi-epitope subunits. The sequence for this particular example appears as residues 108-178 of P3-LIAD (SEQ ID NO: 47). The polypeptide antigens as well as nucleic acids encoding them may be incorpor ated into an adjuvanated form.

As is known in the art, “affinity” is a measure of the strength a particular ligand binds to its partner: Affinities can be measured in different ways. In some embodiments, affinity is measured by a quantitative assay. In some such embodiments, binding partner concentration may be fixed to be in excess of ligand concentration so as to mimic physiological conditions. Alternatively, or additionally, in some embodiments, binding partner concentration and or ligand concentration may be varied. In some such embodiments, affinity may be compared to a reference under comparable conditions (e.g., concentrations).

As used herein, the term “antibody” refers to an immunoglobulin molecule that includes one or more antigen-binding domains that specifically bind to a particular antigen, In some embodiments, the term encompasses any polypeptide or polypeptide complex that includes immunoglobulin structural elements sufficient to confer specific binding.

Exemplary antibodies include, but are not limited to monoclonal antibodies, polyclortal antibodies, and fragments thereof In. some embodiments, an antibody may include one or more sequence elements are humanized, primatixed, chimeric, etc., as is known in the art. In many embodiments, the term “antibody” is used to refer to one or more of the art-known or developed constructs or formats for utilizing antibody structural and functional features in alternative presentation. For example, embodiments, an antibody utilized in accordance with the present invention in a format selected from, but not limited to, intact IgA, or IgG, antibodies; bi- or multi-specific antibodies (e.g., Zybodies®, etc.); antibody fragments such as Fab fragments, Fab* fragments, F(ab*)2 fragments, Fd′ fragments, Fd fragments, and isolated CDRs or sets thereof single chain Fvs; polypeptide-Fc fusions single domain antibodies (e.g.:, shark single domain antibodies such as IgNAR or fragments thereof); cameloid antibodies; masked antibodies (e.g., Probodies®); Small Modular ImmunoPharmaceuticals (“SMPs™”); single chain or Tandem diabodies (TandAb®) VHHs; Anticalins®; Nanobodies® minibodies; BiTE®s; ankyrin repeat proteins or DARPIN®; Avimers®; DARTs; TCR-like antibodies, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glucol, etc.]. In many embodiments, an antibody is or comprises a polypeptide whose amino acid sequence includes one or more structural elements recognized by those skilled in the art as as complementarity determining region (CDR); in some embodiments an antibody is of comprises a polypeptide whose amino acid sequence includes at least one CDR (e.g., at least one heavy chain CDR and/or at least one light chain CDR.) that is substantially identical to one found in a reference antibody. In some embodiments an included CDR is substantially identical to a reference CDR in that it is either identical in sequence or contains between 1-5 amino acid substitutions as compared with the reference CDR. In sonic embodiments an included CDR is substantially identical to a reference CDR in that it shows at least 85%, 86%, 87%, 88%, 89%, 90%, 91%, 92, 93%, 94%, 95%, 90%, 97%, 98%, 99%, or 100% sequence identity with the reference CDR.

in some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added., or substituted as compared with the reference CDR but the included CDR has an amino acid sequence that is otherwise identical with that of the reference CDR. in some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the .)o reference CDR but the included CDR has an amino acid sequence that is otherwise identical with that of the reference CDR. .th some embodiments an included CDR is substantially identical to a reference CDR in that 1-5 amino acids within the included CDR are deleted, added, or substituted as compared with the reference (=DR but the included CDR has an amino acid sequence that is otherwise identical to the reference CDR. in some embodiments, an antibody is or comprises a polypeptide whose amino acid sequence includes structural elements recognized by those Skilled in the art as an immunoglobulin variable domain, In some embodiments, an antibody is a polypeptide protein having a binding domain which is homologous or largely homolaeous to an immunoglobulin-binding domain.

The term “antigen”, as used herein, refers to an agent that binds to an antibody agent. in some embodiments, an antigen binds to an antibody agent and may or may not induce a particular physiological response in an organism. In general, an antigen may be or include any chemical entity such as, fir example, a small molecule, a nucleic acid, a polypeptide, a. carbohydrate, a lipid, a polymer (including biologic polymers [e.g., nucleic acid and/or amino acid polymers] and polymers other than biologic polymers [e.g., other than a nucleic acid or amino acid polymer]) etc. In some embodiments, an antigen is or comprises a polypeptide, In some embodiments, an antigen is or comprises a glycan. Those of ordinary skill in the art will appreciate that, in general, antigen may, be provided in isolated or pure form, or alternatively may be provided in crude form (e.g., together with other materials, for example in an extract such as a cellular extract of other relatively crude preparation of an antigen-containing source). For the vaccines of the present invention it is possible to use the antigenic peptides, DNA (including vectors) encoding the antinenic peptides, and/or mRNA encoding the antigenic peptides. Polypeptides used in serodiagnostics or methods for detecting SARS-COV-2 may he referred to as antigens.

The term “himuniven” may he used synonymously with “antigen” (and vice versa), with “immunogenic peptide”, or as a peptide or polypeptide that induces an immune response, such as a humor al (antibody) or cellular (T cell) response. A subject may be immunized with a SARS-CoV-2 polypeptide antigen. or immunogen, such as one or more peptides or polypeptide antigens disclosed herein, to produce antibodies to the immunogen or to produce T cells which recognize the immunogen when restricted by the MHC.

Polypeptides used in vaccines or to induce immune responses may be referred to as immunogens or immunogenic peptides,

As used herein, a “ligand” can refer to an antibody agent or portion thereof that specifically binds to a target moiety or entity. Typically the interaction between an antigen binding domain and its target is non-covalent. In some embodiments, a target moiety or entity can be of any chemical class including, for example, a carbohydrate, a lipid, a nucleic acid, a metal, a polypeptide, or a small molecule. In some embodiments, an antigen binding domain may be or comprise a polypeptide (or complex thereof). In some embodiments, an antigen binding domain is part of a fusion polypeptide.

Two events or entities are “associated” with one another, as that term is used herein, if the presence, level, and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e,g across a relevant population). In some embodiments, two or more entities are physically “associated” with one another if they interact, directly or indirectly, so that they are and/or remain in physical proximity with one another. In some embodiments, two of more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

It will he understood that the term “bituting”, as used herein, typically refers to a non covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts including where interacting entities or moieties are studied in. isolation or in the context of more complex to systems (e.g., while covalently or otherwise associated with a carrier entity and/or in a biological system or cell).

In general, the term “engineered” refers to the aspect of having been manipulated by the hand of man. For example, a polypeptide is considered to be “engineered” when the polypeptide sequence manipulated by the hand of man. For example, in some embodiment. of the present invention, an engineered polypeptide comprises a sequence that includes one or more amino acid mutations, deletions and/or insertions that have been introduced by the hand of man into a reference polypeptide sequence. In some embodiments, an engineered polypeptide includes polypeptide that has been fused (i.e., covalently linked) to one or more additional polypeptides by the hand of man, to form a fusion polypeptide that would not naturally occur in vivo. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity. Many of the peptide, polypeptide and nucleic acid products disclosed herein are engineered.

The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, arty polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein.

The term “peptide” is generally used to refer to a polypeptide having a length of less than about 100 amino acids, less than about 50 amino acids, less than 20 amino acids, or less to than 10 amino acids. Ian some embodiments, proteins are antibody agents, antibody fragments, biologically active portions thereof, and/or characteristic portions thereof.

Polypeptides or peptides may contain L-amino acids, D-amino acids, or both and may contain any of a variety of amino acid modifications or analogs known in the art. Useful modifications include, c.g., terminal acetylation, amidation, methylation, biotinylation, etc. in some embodiments, proteins may comprise natural amino acids, non-natural amino acids, synthetic. amino acids, and combinations thereof.

The terms “mutation” or and “native” refers to a biological molecule that has not been modified, for example, it can refer to a SARS-CoV-2 isolated from a natural source that has not been modified in the labor atory. It also encompasses SARS-CoV-2 polynucleotides or polypeptides that have not been modified by deletion, substitution or insertion of nucleotides or amino acid residues.

The term “mutation” as used herein indicates any genetic modification of a nucleic acid and/or polypeptide which results in an altered nucleic acid or polypeptide. Mutations include, but are not limited to point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, a nonsense mutation, an insertion, or a deletion of part or all of a gene. In addition, in sonic embodiments of the modified microorganism, a portion of the microorganism genome has been replaced with a heterologous polynucleotide. In some embodiments, the mutations are naturally-occurring. In other embodiments, the mutations are identified and/or enriched through artificial selection pressure.

A mutation or variation may affect the ability of a mutated SARS-CoV-2 to alter the host cell epigenome compared to a wild-type isolate or compared to a different SARS-CoV-2 strain. Such mutations, including RNA modifications involving m6A, m6Am, and 2′-O-me, can affect the structure of the virus, its replication, and the host innate immune responses and innate sensing pathways.

Unless otherwise indicated, the terms “mutated”, “mutant”, “modified”; “altered”, “variant”, and “engineered” are used interchangeably in the present invention as adjectives describing a nucleotide sequence, a nucleic acid, a protein or antigen sequence that is modified to be different from the wild-type nucleotide sequence encoding, for an SARS-COV-2 polynucleotide or polypeptide, By way of example, as used herein in the context of molecules, e.g., nucleic acids, proteins, or small molecules, the term “variant” refers to a molecule that shows significant structural identity with a reference molecule but differs structurally from the reference molecule, e.g. in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function, a nucleic acid may :have a characteristic sequence element comprised of a plurality of nucleotide residues haying designated positions relative to on another in linear or three-dimensional space. In some embodiments, a variant polypeptide or nucleic acid may differ from a reference polypeptide or nucleic acid as a result of one or more differences in amino acid or nucleotide sequence in some embodiments, a variant polypeptide or nucleic acid shows an overall sequence identity with a reference polypeptide or nucleic acid that is at least 80%, 85%, 86%, 87%, 88%, 89%, 90%, 92%, 93%, 94%, 95%, 96%, 97%, or 99%. In some embodiments, a variant polypeptide or nucleic acid does not: share at least one characteristic sequence element with a reference polypeptide or nucleic acid in some embodiments, a reference polypeptide or nucleic acid has one or more biological activities. In some embodiments, a variant polypeptide or nucleic acid shares one or more of the biological activities of the reference polypeptide or nucleic acid.

As used herein, the term “recombinant” is intended to refer to polypeptides that are designed, engineered, prepared, expressed, created, manufactured, and/or or isolated by, recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more to component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.),

An “epitope”, also known as antigenic determinant, is the part of an antigen or immunogen that is recognized by the immune system, such as by antibodies (e.g., IgM, IgG, IgE, IgA, etc), B cells, or T cells. The epitopes of protein antigens are divided into two categories, conformational epitopes and linear epitopes, based on their structure and interaction with the paratope. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontinuous amino acid residues. In contrast, a linear epitope is formed by the 3-D conformation adopted by the interaction of contiguous amino acid residues. T cell epitopes are presented on the surface of an antigen-presenting cell, where they are bound to major histocompatibility complex (MHC) molecules. In humans, professional antigen-presenting cells are specialized to present MHC class II peptides, whereas most nucleated somatic cells present MHC class 1 peptides. T cell epitopes presented by MTC class molecules are typically peptides between 8 and amino acids in length, whereas MHC class II molecules present longer peptides, 13-17 amino acids in length, and non-classical MHC molecules also present non-peptidic epitopes such as glycolipids.

As used herein, the term “vector” refers to a nucleic acid molecule capable of transporting another nucleic acid to which it has been linked, Non-limiting examples of expression vectors include plasmid vectors, transposon vectors, cosmid vectors, and viral derived vectors (e.g., any adenoviral derived vectors (AV), cytomegaloviral derived (CMV) vectors, simian viral derived (SV40) vectors, adeno-associated virus (AAV vectors, lentivirus vectors, and retroviral vectors). In some embodiments, the expression vector is a to viral vector, One type of preferred vector is a “plasmid”, which refers to a circular double stranded DNA loop into which additional DNA segments may be ligated. Another type of preferred vector is a viral vector, wherein additional DNA segments may be ligated into the viral genome. Certain vectors are capable of autonomous replication in a host cell into which they are introduced (cg, bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression rectors.”

mRNA and alphtmfrus platforms. In other embodiments, a sequence encoding a SARS-CoV-2 peptide or polypeptide antigen may be incorpor ated into a mRNA vaccine or into a self-replicating RNA platform, such as into an alpha-virus ‘platform’, see Wikipedia “mRNA vaccine” hypertext transfer protocol secure://en.wikipediaorewiki/MRNA_vaccine, last accessed Nov. 13, 2022; Park KS, et al., Non-vitral COVID-19 vaccine delivery system; ADAVANCED DRUG D_{ELIVERY} REVIEWS2020, 169; 137-5; and Maria Cristina Ballesteros-Briones, et al, A new generation of vaccines based on alphavirus self-amplifying RNA, CURR. OPIN VIROL.2020, 44: 145-153, both incorpor ated by reference;

Codon modification. This polynucleotides encoding SARA -COV-2 peptides or polypeptides (e.g., immunogenic or antigenic, peptides or polypeptides) may further comprise replacing at least one degenerate codon in the nucleic acid sequence which encodes the SARS-COV-2 polypeptide or an immunogenic fragment or variant thereof, with a different codon encoding the same amino acid, which different codon increases GC content. reduces RNA hairpin formation, increases expression SARS-CoV-2 polypeptides, polypeptide fragments or variants, or increases the stability of the platform, vector or coding mRNA or DNA. In one embodiment the nucleic acid sequences of a vector or other platform encoding a SARS-COV-2 peptide or polypeptide are codon modified based on the codon usage in the host cell a human cell or other animal vector or carrier or animal susceptible to infection including cats, dogs, tigers, liars, pangolins, minks, camels, dromedary camels, and non-human primates; see Mahdy, et al., An Overview, of SARS-CoV-2 and Animal Infection, FRONT VET Sci, 202, 7:596391, incorpotated by reference) or based on decreasing or increasing their GC content. Various functions and methods for codon modification may be used including those described by, and incorpor ated by reference to Hanson, G., Coller, J. Codon optimality, bias and usage in transletion and mRNA deay. NAT MOL CELL BIOL 19, 20-30 (2018). https://doi .org/10.1038/mm.2017.91.; hypertext transfer protocol secure://en.wkipedia.org/wiki/Codon_usage_bias#Effect_on_transcription_or_gene_expressi on (last accessed Jun. 7, 2022) and by the references cited therein.

Molecular biological techniques. Standard techniques may be used for production or handling of recombinant DNA, oligonucleotide synthesis, and tissue culture and transformation (e.g., electropor ation, lipofection). Enzymatic reactions and purification techniques may be performed according to manuacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et ed., Molecular Cloning; A Labor atory Manual 2nd ed., Cold Spring Harbor Labor atory Press, Cold Spring Harbor, N. Y, (1989)), which is incorpor ated. herein by reference for any purpose.

As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting, or determining degree of association between the binding agent and its partner; in some embodiments, specific binding is assessed by detecting or determining degree of dissociation of a binding agent-partner complex; in some 3(i embodiments, specific binding is assessed by detecting or determining ability of the binding agent to compete an alternative interaction between its partner and another entity. In some embodiments, specific binding is assessed by performing such detections or determinations across a range of concentrations.

As used herein ELISA refers to enzyme-linked immunoabsorbent assay. Various kinds of ELISA may he performed including, but not limited to, direct ELISA, sandwich ELBA, competitive ELISA, or reverse ELISA. An ELISA may employ enzymatic markers such as OPD (o-phenvienediamine dihydrochloride), TMB (3,3′,5,5′-tetramethylbenzidine), ARTS (2,2′-Azinobis [3-ethylbenzothiazoline-6-sulfonic acid]-diammonium salt), or PNPP (p-Nitrophenyl Phosphate, Disodium Salt). Various types of ELISA and ELISA materials are known are the art and incorpor ated by reference to Engvall, E (1972, Nov. 22), Enzyme-linked immunosorbent assay, Elisa. JOURNALOF IMMUNOLOGY. 109(1): 129-135. ISSN 0022-1767. PAID 4113792; Crowther. J. R. (1995). Chapter 2: “Basic Principles of ELISA”, ELISA: Theory and Practice. METHODS IN MOLECULAR BIOLOGY. Vol. 43. Humana Press, pp. 35-62. Doi 10.1385/0-89603-279-5:1. ISBN 0896032795, PMID 7655571; and to hypertext transfer protocol secute.//en.wikipedia,org/wiki/ELISA (last accessed Nov. 18, 2022). The peptide and polypeptide antigens disclosed herein may also be used in radioimmunoassays, ELISpot, lateral flow, or multiplex assays and other types of immunoassays for detecting antibodies to SARS-CoV-2.

As used herein, the term “subject” refers an organism typically a mammal (e.g., a human, in some embodiments including prenatal human forms). In some embodiments, subject is suffering from a relevant disease, disorder or condition. In some embodiments, a subject is susceptible to a disease, disorder, or condition, in some embodiments a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In sonic embodiments, a subject is a patient, in some embodiments, a subject is an individual to whom diagnosis and/or therapy is and/or has been administered.

Subjects include humans as well as other animals susceptible to infection with SARS-CoV-2 or subject to being vectors or earners of this virus.

As used herein, the term “allministration” typically refers to the administration of a composition to a subject or system to achieve delivery of an agent that is, or is included in, 3(i the composition. Those of ordinary skill in the art will be aware of a variety of routes that may, in appropriate circumstances, be utilized for administration to a subject, for example a human. For example, in some embodiments, administration may be ocular, or al, parenteral, topical, injection, aerosol spray, aqueous mist, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, oculo-nasal administration using aerosol, intranasal, intraperitoneal, intrathecal, intravenous, intraventricular, within a specific organ (e. intrahepatic), mucosal, nasal, or al, rectal, subcutaneous, sublingual, topical, tracheal (e.g., by intratracheal instillation), vaginal, vitreal, etc. In a preferred embodiment, administration is by injection (e.g, intravenous, subcutaneous, intramuscular, intraorbital, intraocular, intradermal, and/or intraperitoneal). in some embodiments, administration may involve only a single dose. In some embodiments, administration may involve application of a fixed number of doses. In to some embodiments, administration may involve dosing that is intermittent (e.g., a plurality of doses separated in time) and/or periodic (e.g., individual doses separated by a common period of time) dosing., in some embodiments, administration may involve continuous dosing (cg., perfusion) for at least a selected period of time. Also, administration can be made at any age depending on the conditions, for instance to newborns to 4 year olds, to 5 to 11 year olds, to 12 to 17 year olds, to 18 year olds to 4year olds, and/or to over 65 years olds. Administration can be to anatomical females and/or anatomical mates. Administration can be to subjects with a weakened immune system, that are moderately to severely immunocompromised, and/or suffering from an underlying medical condition. Underlying medical conditions that have been associated with increased severity of the progression and severity of SARS-CoV-2 mediated conditions or symptoms include heart disease, lung disease, and diabetes.

As used. herein, the terms “treat”, “treatment”, and “treating” refer to both therapeutic and prophylactic treatments. For example, therapeutic treatments includes the reduction or amelior ation of the progression of SARS-CoV-2 or symptoms associated therewith, reduction or amelior ation of the severity of SARS-CoV-2 or symptoms associated therewith, and/or reduction or amelior ation or the duration of SARS-CoV-2 mediated conditions or symptoms associated therewith, or the amelior ation of one or more symptoms of exposure to SARS-CoV-2 mediated conditions, resulting from the administration of one or more therapies (e,g., one or more biological therapeutic agents such as SARS-CoV-2 immunogens, antibodies to SARS-CoV-2 antigens, or T cells or, other immunocytes recognizing SARS-CoV-2 antigens or epitopes). On average it takes 5-6 days from when someone is infected with the virus for symptoms to show, however it can take up to 14 days.

The terms “proplodaxis” or “prophylactic use” and “prophylactic treatment” used herein, refer to any procedure whose purpose is to prevent, rather than treat or cure a disease. Although the terms “prevent”, “prevention”, and “preventing” may include complete eradication of conditions and/or symptoms associated with SARS-CoV-2, as used herein, the terms “prevent”, “prevention”, and “preventing” refer to the reduction in the risk of acquiring or developing a given condition and/or symptoms associated with SARS-CoV-2, or the reduction or inhibition of the recurrence or said condition in a subject or symptoms associated therewith, such as a human, who is not previously afflicted with SARS-COV-2, but who has been or may be near or exposed to SARS-CoV-2.

In some embodiments, the terms “prevent”, “prevention”, and “preventing” refer to the reduction in the risk of acquiring or developing new or ongoing symptoms that can last in weeks or months after first being infected with SARS-CoV-2 (referred to as post-COVID conditions), reduction in the risk of acquiring or developing multi-organ effects or autoimmune conditions that can last for weeks or months after being infected with SARS-CoV-2, and/or reduction in the risk of acquiring or developing post-intensive care syndrome (PICS). Multi-organ effects can affect many, if not all, body systems, including heart, lung, kidney, skin, and brain functions, Autoimmune conditions occur when the immune system attacks healthy cells in your body by mistake, causing inflammation (swelling) or tissue damage in the affected parts of the body, A serious multiorgan effect, especially amonast children, is multisystem inflammatory syndrome (MIS). MIS typically manifests during or immediately after a COVID-19 infection. PICS refers to health effects that begin when a person is in an intensive care unit (ICU) and can remain after a person returns home. These effects can include severe weakness, problems with thinking and judgment, and post-traumatic stress disorder (PTSD),

The U.S. Center for Disease Control (hypertext transfer protocol secure://www.cdc.gov/coronavirus/2019-ncov/symptoms-testing/symptoms.html, last accessed Nov. 9, 2021) recognize the following symptoms associated with SARS-CoV-2 infection, but it is understood that the present invention is not limited to these symptoms and includes other symptoms hereafter identified as associated with SARS-CoV-2: fever, chills, cough or dry cough, tiredness (i.e., fatigue), muscle or body aches and pains, sore throat, diarrhea, nausea, vomiting, loss of taste and/or smell, and/or headache, congestion or runny nose. Other symptoms include conjunctivitis, a rash on skin, and/or discolor ation of fingers and/or toes. Serious symptoms include: difficulty breathing and/or shortness of breath, chest lain and/or pressure, and loss of speech of movement.

The U.S. Center for Disease Control (hypertext transfer protocol secure://www.cdc.gov/coronavirus/2019-ncov/long-term-effects/index.html, last accessed Nov. 9, 2021) recognize the following, symptoms associated with SARS-CoV-2 infection as post-COVID conditions, hat it is understood that the present invention is not limited to these symptoms and includes other symptoms hereafter identified as post-COVII) conditions: difficulty breathing or shortness of breath, tiredness or fatigue, symptoms that get worse after physical or mental activities (also known as post-exertional malaise), difficulty thinking or concentrating (sometimes referred to as “brain fog”), cough, chest. or stomach pain, headache, fast-beating or pounding heart (also known as. heart palpitations), joint or muscle pain, pins-and-needles feeling, diarrhea, sleep problems, fever, dizziness on standing (lightheadedness), rash, mood changes, change in smell or taste, and/or changes in menstrual period cycles

As used herein, an “effective amount” refers to an amount sufficient to elicit the desired biological response. In the present invention the desired biological response is to inhibit the replication of SARS-CoV-2, to reduce the amount of, or viability of, SARS-CoV-2 or to reduce or amelior ate the severity, duration, progression, or onset of an SARS-CoV-2 infection, prevent the advancement of an SARS-CoV-2 infection, prevent the recurrence, development, onset or progression of a symptom associated with an SARS-CoV-2 virus infection, or enhance or improve the prophylactic or therapeutic effect(s) of another therapy used against SARS-CoV-2 infections. The precise amount of compound administered to a subject, such as a human, will depend on the mode of administration, the type and severity of the infection and an the characteristics of the subject, such as general health, age, sex, body weight, genetic and immunological background, and tolerance to biological agents or drugs,

The skilled artisan will he able to determine appropriate dosages of a biological agent (“biologic”), such a SARS-CoV-2 protein or immunogen, a SARS-CoV-2 polynucleotide, a SARS-CoV-2 mRNA antigen, antibodies recognizing SARS-CoV-2 antigens, and immune cells directed against SARS-CoV-2, depending on these and other factors. Those of ordinary skill in the art will appreciate that, in some embodiments, an effective amount may be formulated and/or administered in a single dose. In some embodiments, a therapeutically effective amount may be formulated and/or administered in a plurality of doses, for example, as pan of a dosing regimen.

As used herein, the term “pharmaceutical composition” or “thentpeutic agent” in general refers to any agent that elicits a desired pharmacological effect when administered to an organism. In particular, these terms refer to a composition in which an active agent (e.g., as SARS-CoV-2 biologic as described herein) is formulated together with one or more pharmaceutically acceptable carriers, in some embodiments, the composition is suitable for administration to a human or animal subject. hi some embodiments, an agent is considered. to be a therapeutic if it demonstrates a statistically significant effect across an. appropriate population. In some embodiments, the appropriate population may be a population of model organisms. In some embodiments, an appropriate population may be defined by various criteria., such as a certain age group, gender, genetic background, preexisting clinical conditions, ex. In some embodiments, a therapeutic agent is a substance that can be used to alleviate, amelior ate, relieve, inhibit, prevent, delay onset of, reduce severity of, and/or reduce incidence of one or more symptoms or features of a disease, disorder, and/or condition. In some embodiments, the active agent is present in unit dose amount appropriate for administration. in a therapeutic regimen that shows a statistically significant probability of achieving a predetermined therapeutic effect when administered to a relevant population. A SARS-COV-2 vaccine may be formulated using an effective amount of one or more peptide, polypeptide, or nucleic acid products or constructions disclosed herein. In one embodiment, the ability to confer protective immunity against a SARS-CoV-2 serotype would indicate that at least 75% of subjects vaccinated with the vaccine prepared in accordance. with the present invention would be protected against disease caused by at least one strain of such serotype.

An objective of such a vaccine is to protect or reduce the severity of SARS-CoV-2 infection or its symptoms by at least 50, 60, 70, 88, 90 or 95% or to provide complete resistance to reinfection or challenge with the same or a different strain of SARS-CoV-2. In same embodiments of the present invention, the vaccine protects or reduces the severity of SARS-CoV-2 infection or its symptoms by at least 50, 60, 70, 88, 90 or 95% or to provide complete resistance to reinfection or challenge regardless of the SARS-CoV-2 genetic variations.

Moreover, a vaccine advantageously can provide potent cross-protective immunity and conkr unique advantages to vaccinated subjects as disclosed herein.

The proteolytic activation of the S spike protein is instrumental for coronaviridae to gain host-cell entry, For SARS-CoV-2 the S protein display particular sequences containing a Furin and a TMPRSS2 cut sites as well as a membrane fusion peptide {FP}. These sequences located at the S1/S2/S′2 Junction subunits are involved in the proteolytic activation associated to the virus enhanced target cells invasion and infectivity.

These sequences were not expiated so :tar in immunity studies or for designing prophylactic vaccines to COVID19.

The patient-centered study of 500 PCR-confirmed COVID19 individuals we performed showed specific IgG antibody response against a number of epitopes present at the S1/S2 junction sequence.

This response is highly associated (p=0.0001) to the asymptomatic and mild forms of COVID19 and persists several months', which reveals a protective immunity.

Mice immunization with various combination of peptides bearing the aforementioned epitopes yielded specific immune responses.

The term “phartmucentically acceptable cattier”, as used herein, generally has its art-recognized meaning of a pharmaceutically acceptable material, composition or carrier, such as a liquid or solid filler, stabilizer, dispersing agent, suspending agent, diluent, excipient, thickening agent, solvent or encapsulating material, involved w carrying or transporting a compound useful within the invention within or to the patient such that it may perform its intended function. A pharmaceutically acceptable carrier, which may be admixed or compounded with a SARS-CoV-2 biologic as described herein, may contain inert ingredients which do not unduly inhibit the biological activity of the compounds. The pharmaceutically acceptable carriers should be biocompatible, e.g., non-toxic, non-inflammatory, non immunogenic or devoid of other undesired reactions or side-effects upon the administration to a subject Standard pharmaceutical formulation techniques can be employed,

Typically, such polypeptide antigens are carried or transported from One organ or portion of the body, to another organ, or portion of the body. Each carrier must be “acceptable” in the sense of being compatible with the other ingredients of the formulation, including the compound useful within the invention, and not injurious to the patient. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. The term “pharmaceutically acceptable carrier” may, further include a pharmaceutically acceptable salt of the compound useful within the invention. Other additional ingredients that may be included in the pharmaceutical compositions used in the practice of the invention, are described, for example, in Remington's Pharmaceutical Sciences (Genaro, Ed., Mack Publishing Co., 1985, Easton, Pa.), which is incorpor ated herein by reference, The “pharmaceutically acceptable carrier” is useful for the preparation of a pharmaceutical composition that is: generally compatible with the other ingredients of the composition, not deleterious to the recipient, and neither biologically nor otherwise undesirable, “A pharmaceutically acceptable carrier” includes one or more than one carrier. Embodiments include carriers for topical, ocular, parenteral, intravenous, intraperitoneal intramuscular, sublingual, nasal or or al administration. “Pharmaceutically acceptable carrier” also includes agents for preparation of aqueous dispersions and sterile powders for injection or dispersions.

The pharmaceutically acceptable carrier, adjuvant, or vehicle, as used herein, includes any and all solvents, diluents, or other liquid vehicle, dispersion or suspension aids, surface active agents, isotonic agents thickening or emulsifying agents, preservatives solid binders, lubricants and the like, as suited to the particular dosage form desired. Multiple oils and combinations thereof are suitable for use for administering an immunogen or vaccine as disclosed herein. These oils include, without limitations, animal oils, vegetable oils, as well as non-metabolizable oils, Non-limiting examples of vegetable oils suitable in the instant to invention are corn oil, peanut oil, soybean oil, coconut oil, olive oil, and phytosqualane. Non-limiting exam of animal oils is squalane. Suitable non-limiting examples of non-metabolizable oils include light mineral oil, straight chained or branched saturated oils, ramified oils, and the like.

Emulsifiers suitable for use in a composition or vaccine (e.g., as an emulsion) as disclosed herein include natural biologically compatible emulsifiers and non-natural synthetic surfactants. Biologically compatible emulsifiers include phospholipid compounds or a mixture of phospholipids, Preferred phospholipids are phosphatidylcholines (lecithin), such as soy or egg lecithin. Lecithin can be obtained as a mixture of phosphatides and triglycerides by water-washing crude vegetable oils, and separating. and drying the resulting hydrated gums. A refined product can be obtained by fractionating the mixture for acetone insoluble phospholipids and glycolipids remaining after removal of the triglycerides and vegetable oil by acetone washing. Alternatively, lecithin can be obtained from various commercial sources. Other suitable phospholipids include phosphatidylglycerol, phosphatidylinositol, phosphatidylserine, phosphatidic acid, cardiolipin, phosphatiylethanolamine, lysophosdylcholine, lysophosphatidylserine, sophosphatidylinositol, and hysophosphatidylethanolamine. The phospholipids may be isolated from natural sources or conventionally synthesized.

Remington's Pharmaceutical Sciences, Twenty third Edition. Academic Press, 2020) discloses various carriers used in formulating pharmaceutically acceptable compositions and in known techniques for the preparation thereof. Except insofar as any conventional carrier medium is incompatible with the compounds described herein, such as by producing any undesirable biological effect or otherwise interacting. in a deleterious manner with any other component(s) of the pharmaceutically acceptable composition, its use is contemplated to be within the scope of this invention.

Some examples of materials which can serve as pharmaceutically acceptable caraiers include, but are not limited to, ion exchangers, alumina, aluminum stearate, lecithin, serum proteins (such as human serum albumin), buffer substances (such as tween 80, phosphates, glycine, sorbic acid, or potassium so/bate), partial glyceride mixtures of saturated vegetable fatty acids, water, salts or electrolytes (such as protamine saltine, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylceilulose, hydroxypropyl methylcellulose, wool fat, sugars such as lactose, glucose and sucrose; starches such as corn starch and potato to starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidains can also be present in the composition according to the judgment of the formulator.

As used herein, the term “pharmaceutically (weep able salt” generally has its part-recognized meaning and refers to derivatives of the compounds provided herein wherein the parent compound is modified by converting an existing acid or :rase moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds provided herein include the conventional non-toxic salts of the parent compound formed, for example, from non-toxic inorganic or organic acids. The pharmaceutically acceptable salts of the compounds provided herein can be synthesized from the parent compound which contains a basic or acidic moiety by conventional chemical methods.

Generally, such salts can be prepared by combining the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile may be used. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorpor ated herein by reference in its entirety,

As used herein, the term “formulation” generally has its art-recognized caning and refers to the combination of all the substances that go into its composition. In the formulation, at least two kinds of compounds are distinguished; the active principle and the excipients.

As used herein. the “active component,” “active sub8tance,” or “active principle” generally has its art-recognized meaning and refers to each of the components of a composition or formulation which has a therapeutic effect. This substance is often in very low proportion in the drug compared to the excipients. This can be a chemically defined pure substance (more or less wrongly qualified as a “molecule”) or a mixture of several chemically similar substances (isomers, for example) or even a substance defined by its mode of production. Of course, it in the context of the present invention the “drug” need not he small molecule, but rather refers to the immunogenic peptides, polypeptides,-stein subunits, and nucleic acids as set forth herein.

As used herein, the term “excipient” generally has its art recognized meaning and refers to any substance other than the active principle in a drug, cosmetic or food. Its addition is intended to confer a given consistency, or other particular physical or taste characteristics, to the final product, while avoiding any interaction, particularly chemical, with the active principle. An excipient is therefore not defined by a particular chemical composition but by its use, which results from its physicochemical properties which make it suitable for fulfilling its role of excipient. Thus, an excipient includes physiologically compatible additives useful in preparation of a pharmaceutical composition. Examples of pharmaceutically acceptable carriers and excipients can, for example, be found in Remington Pharmaceutical Science, 16^{th}Ed.

The term “about”, when used herein in reference to a value, refers to a value that is similar, in context to the referenced value. in general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 2%, 1%, 0.5% or less of the referred value.

Unless otherwise defined, all technical and scientific terms used herein have the same meaning as commonly understood by one of ordinary skill in the art to which this disclosure belongs. In case of conflict, the present specification, including definitions, will control.

Unless stated otherwise, all percentages, parts, ratios, etc;, are by weight.

When an amount, concentration, or other value or parameter is given as a range, or a list of upper and lower values, this is to be understood as specifically disclosing all ranges formed from any pair of any upper and lower range limits, regardless of whether ranges are separately disclosed. Where a range of numerical values is recited herein, unless otherwise stated, the range is intended to include the endpoints thereof, and all integers and fractions within the range. Also, all values and subranges within a numerical limit or range are specifically included as if explicitly written out.

Further, unless otherwise explicitly stated to the contrary, when one or multiple ranges or lists of items are provided, this is to be understood as explicitly disclosing any single stated value or item in such range or list, and any combination thereof with any other individual value or item in the same or any other list.

As used herein, the terms “comprises,” “comprising,” “includes,” “including,” “has,” “having” or any other variation thereof, are intended to cover a non-exclusive inclusion. For example, a process, method, article, or apparatus that comprises a. list of elements is not necessarily limited to only those elements but can include other elements not expressly listed or inherent to such process, method, article, or apparatus.

The transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause:, other elements are not excluded from the claim as a whole.

The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Optional additives as defined herein, at a level that is appropriate for such additives, and minor impurities are not excluded from a composition by the term “consisting essentially of”.

As used herein, the Phrases “selected from the group consisting of,” “chosen from,” and the like include mixtures of the specified materials.

Further, unless expressly stated to the contrary, “or” and “and/or” refers to an inclusive and not to an exclusive. For example, a condition A or B, or A and/or B, is satisfied by any one of the following: A is true (Or present) and B is false (or not present), A is false (or not present) and B is true (or present), and both A and B are true (or present).

The use of “a” or “an” to describe the various elements and components herein is merely for convenience and to give a general sense of the disclosure. This description should be read to include one or at least one and the singular also includes the plural unless it is obvious that it is meant otherwise.

In the context of the present description, all publications, patent applications, patents and other references mentioned herein, if not otherwise indicated, are explicitly incorpor ated by reference herein in their entirety for all purposes as if fully set forth, and shall be in considered part of the present disclosure in their entirety.

In this disclosure, we disclose immunoactive peptides, polypeptides and protein subunits spanning the S protein S1/S2/S′2 junction, These products are suitable for use in precision vaccines against SARS-COV-2 as well as an ELISA assay for the tracking of protective immunity or infection by a variety of different SARS-COV-2 strains,

Immittiottetive Peptides. In the design of novel vaccines for SARS-CoV-2 a number immunoactive peptides (herein also referred to as antigenic peptides) were designed. For example, using a mix of rational and computational design that predicts the proteasome/immunoproteasome generated peptides putatively binding to MUIC antigen presenting molecules and/or potential B cell epitopes, a first set of peptides were identified, which are derived from the S protein S1/S2 subunits junction sequence which are exclusive to SARS-CoV-2.

The first set of antigenic peptides are based an P1, P2, P3, P4, P5, P9, P19, P22, P23, P24 (SEQ ID NOs: 1-10) as shown below. Underlined sequences show residues of SARS-2 COV-2 S protein located at the junction of S1/S2. These specific sequences contain epitopes surrounding and/or overlapping the furin proteolytic cut cite PRRAR.

| | | SEQ |
|---|---|---|
| Peptide | | ID |
| Name | Peptide Sequence | NO: |
| P1 | *GGGGSQTNSPRRARSVASQSI | 1 |
| P2 | *GGGGSQTNSPRRARSVASQSIGGGGSQTNSPRRA | 2 |
| | RSVASQSI | |
| P3 | *GGGGSQINSPRRARSVASQSIGGGGSQTNSPRRA | 3 |
| | RSVASQSIGGGGSQTNSPRRARSVASQSI | |
| P4 | *GGGGSQCNSPRRARSVASQCI | 4 |
| P5 | *GGGGSQCNSPRRARSVASQCIGGGGS* | 5 |
| P19 | *GGGGSSPRRARSVAS | 6 |
| P21 | *GGGGSSPRRARSVASGGGGSSPRRARSVASGGGG | 7 |
| | SSPRRARSVAS | |
| P22 | *GGGGSQTQTNSPRR | 8 |
| P23 | *GGGGSQTNSPRRAR | 9 |
| P24 | *GGGGSNSPRRARSV | 10 |

*designates an optional biotin functionality

In some embodiments, the antigenic peptides of SEQ ID NO: 1-10 can be used without the presence of the GGGGS linker sequence(s), Specifically, the following SEQ ID NOs: 11-15 can be mentioned:

| Peptide Sequence | SEQ ID NO: |
|---|---|
| QTNSPRRARSVASQSI | 11 |
| SPRRARSVAS | 12 |
| QTQINSPRR | 13 |
| QTNSPRRAR | 14 |
| NSPRRARSV | 15 |

Thus, a second set of antigenic-peptides for use in the preparation of a vaccine of the present invention can include one or more polypeptides of SEQ ID NOs; 1-15,

A third set of antigenic peptides for use in the preparation of a vaccine of the present invention can include one or more polypeptides shown below. Underlined sequences show residues of SARS-CoV-2. S protein located at the junction of S1/S2. These specific sequences contain epitopes surrounding and/or overlapping the furin proteolytic cut cite PRRAR.

| SEQ | | |
|---|---|---|
| ID | Peptide | |
| NO: | Name | Peptide Sequence |
| 1 | P1 | *GGGGSQTNSPRRARSVASQSI |
| 16 | P1-B7 | SPRRARSVASQSI |
| 2 | P2 | *GGGGSQTNSPRRARSVASQSIGGGGSQTNSPRRAR |
| | | SVASQSI |
| 3 | P3 | *GGGGSQTNSPRRARSVASQSIGGGGSQTNSPRRAR |
| | | SVASQSIGGGGSQTNSPRRARSVASQSI |
| 4 | P4 | *GGGGSQCNSPRRARSVASQCI |
| 5 | P5 | *GGGGSQCNSPRRARSVASQCIGGGGS* |
| 6 | P19 | *GGGGSSPRRARSVAS |
| 17 | P19-B8 | SPRRARSV |
| 18 | P19-B7 | SPRRARSVA |
| 7 | P21 | *GGGGSSPRRARSVASGGGGSSPRRARSVASGGGGS |
| | | SPRRARSVAS |
| 8 | P22/e | *GGGGSQTQTNSERR |
| 13 | P22/v | QTQTNSPRR |
| 9 | P23/e | *GGGGSQTNSPRRAR |
| 14 | P23/v | QTNSPRRAR |
| 15 | P24 | NSPRRARSV |
| 19 | PepF25 | IAIPTNFTI |
| 20 | PepF24 | NSIAIPTNF |
| 21 | PepF23 | SVAYSNNSI |
| 22 | FepF22 | SIIAYTMSL |
| 23 | PepF21 | SQSIIAYTM |
| 24 | PepF20 | VASQSIIAY |
| 25 | PepF20-/ | SVASQSIIAY |
| | A26- | |
| | B15 | |
| 26 | PepF19 | RSVASQSII |
| 27 | PepF10 | IPINFTI |
| 28 | PepF7 | IGAGICASY |
| 29 | PepF6 | CDIPIGAGI |
| 30 | PepF5 | ECDIPIGAG |

*designates an optional biotin functionality

The polypeptides of SEQ ID NOs: 11, 12, and/or 15 may also be included in the third set of antigenic peptides.

In an embodiment of the present invention are immunoactive peptides that comprise at least a peptidic farin cleavage site sequence with RRAR tSEQ ID NO 1061 motif each of which can be used in the preparation of a vaccine in accordance with the present invention. The peptides include (*represents an optional biotin group):

A peptide of SEQ ID NOS: 1-7, 9-12, 14-15 above

Variants of Peptide 3:
(SEQ ID NO: 31)
SQTNSPRRARSVASQSI
(SEQ ID NO: 1)
GGGGSQTNSPRRARSVASQSI
(SEQ ID NO: 108)
GGGGSQTNSPRRARSVASQSIGGGG
(SEQ ID NO: 2)
GGGGSQTNSPRRARSVASQSIGGGGSQTNSPRRARSVASQSIGGGGSQTN
SPRRARSVASQSI
Variants of Peptide 21
(SEQ ID NO: 18)
SPRRARSVA
(SEQ ID NO: 32)
GGGGSPRRARSVA
(SEQ ID NO: 33)
GGGGSPRRARSVAGGGG
(SEQ ID NO: 7)
GGGGSSPRRARSVASGGGGSSPRRARSVASGGGGSSPRRARSVAS
Variants of Peptide 19
(SEQ ID NO: 34)
SSPRRARSVAS
(SEQ ID NO: 6)
GGGGSSPRRARSVAS
Variants of Peptide 1
(SEQ ID NO: 31)
SQTNSPRRARSVASQSI
(SEQ ID NO: 1)
GGGGSQTNSPRRARSVASQSI
(SEQ ID NO: 1)
Biotin-GGGGSQTNSPRRARSVASQSI
Variants of Peptide 2
(SEQ ID NO: 35)
SQTNSPRRARSVASQSIGGGGSQTNSPRRARSVASQSI
(SEQ ID NO: 2)
GGGGSQTNSPRRARSVASQSIGGGGSQTNSPRRARSVASQSI
(SEQ ID NO: 2)
Biotin-GGGGSQTNSPRRARSVASQSIGGGGSQTNSPRRARSVASQSI
Variants of Peptide 3
(SEQ ID NO: 3)
GGGGSQTNSPRRARSVASQSIGGGGSQTNSPRRARSVASQSIGGGGSQTN
SPRRARSVASQSI
(SEQ ID NO: 3)
Biotin-GGGGSQTNSPRRARSVASQSIGGGGSQTNSPRRARSVASQSIG
GGGSQTNSPRRARSVASQSI
Variants of Peptide 4
(SEQ ID NO: 36)
SQCNSPRRARSVASQCI
(SEQ ID NO: 4)
GGGGSQCNSPRRARSVASQCI
(SEQ ID NO: 4)
Biotin-GGGGSQCNSPRRARSVASQCI
Variants of Peptide 5
(SEQ ID NO: 38)
SQCNSPRRARSVASQCIGGGGS
(SEQ ID NO: 5)
Biotin-GGGGSQCNSPRRARSVASQCIGGGGS-Biotin
Variants of Peptide 19
(SEQ ID NO: 34)
SSPRRARSVAS
(SEQ ID NO: 6)
GGGGSSPRRARSVAS
(SEQ ID NO: 6)
Biotin-GGGGSSPRRARSVAS
Variants of Peptide 21
(SEQ ID NO: 34)
SSPRRARSVAS
(SEQ ID NO: 7)
GGGGSSPRRARSVASGGGGSSPRRARSVASGGGGSSPRRARSVAS
(SEQ ID NO: 7)
Biotin-GGGGSSPRRARSVASGGGGSSPRRARSVASGGGGSSPRRARSV
AS
Variants of Peptide AGU-P1
(SEQ ID NO: 11)
QTNSPRRARSVASQSI
(SEQ ID NO: 1)
*GGGGSQTNSPRRARSVASQSI
Variants of Peptide AGU-P2
(SEQ ID NO: 11)
QTNSPRRARSVASQSI
(SEQ ID NO: 2)
*GGGGSQTNSPRRARSVASQSIGGGGSQTNSPRRARSVASQSI
Variants of Peptide AGU-P3
(SEQ ID NO: 11)
QTNSPRRARSVASQSI
(SEQ ID NO: 3)
*GGGGSQTNSPRRARSVASQSIGGGGSQTNSPRRARSVASQSIGGGGSQT
NSPRRARSVASQSI
Variants of Peptide AGU-P4
(SEQ ID NO: 37)
QCNSPRRARSVASQCI
(SEQ ID NO: 4)
*GGGGSQCNSPRRARSVASQCI
Variants of AGU-P5
(SEQ ID NO: 37)
QCNSPRRARSVASQCI
(SEQ ID NO: 5)
*GGGGSQCNSPRRARSVASQCIGGGGS*
Variants of AGU-P19
(SEQ ID NO: 12)
SPRRARSVAS
(SEQ ID NO: 6)
GGGGSSPRRARSVAS
Variants of AGU-P21
(SEQ ID NO: 12)
SPRRARSVAS
(SEQ ID NO: 7)
GGGGSSPRRARSVASGGGGSSPRRARSVASGGGGSSPRRARSVAS

In an embodiment of the present invention are antigenic peptides that are free of the RRAR motif (SEQ ID NO: 106) but incorpor ates into or overlaps the motif KPSKRSFIED (SEQ LD NO: 114) each of which can be used in the preparation of a vaccine in accordance with the present invention. The GGGGS residues are underlined. These are used as linkers to engineer peptides/polypeptides where epitopes retain their tertiary structures and immunogenicities. Such sequences can be homopolymer red and appear more than once or is in a given peptide/polypeptide, for example, as shown for P3 (SEQ ID NO: 39) and P3-L AD (SEQ ID NO: 47).

In an einbodiment of the present invention are antigenic peptides selected from:

| SEQ ID NO: 113 | |
|---|---|
| P4/TMP | PDPSKPSKR |
| SEQ ID NO: 115 | |
| P4/TMP bn | *GGGGSPDPSKPSKR |
| SEQ ID NO: 116 | |
| P4/TMP-Pol3 | GGGGSPDPSKPSKRGGGGSPDP |
| | SKPSKRGGGGSPDPSKPSKR |
| SEQ ID NO: 117 | |
| P4/TMP-Pol3- | *GGGGSPDPSKPSKRGGGGSPDP |
| bn | SKPSKRGGGGSPDPSKPSKR |
| SEQ ID NO: 112 | |
| P3/TMP | IEDLLFNKV |
| SEQ ID NO: 118 | |
| P3/TMP -bn | *GGGGSIEDLLFNKV |
| SEQ ID NO: 111 | |
| P2/TMP | RDLICAQKF |
| SEQ ID NO: 119 | |
| P2/TMP -bn | *GGGGSRDLICAQKF |
| SEQ ID NO: 110 | |
| P1/TMP | ICAQKFNGL |
| SEQ ID NO: 120 | |
| P1/TMP-bn | *GGGGSICAQKFNGL |

(*represents an optional biotin group)

In an embodiment of the present .invention are inuminoactive peptides that comprise die peptidic sequence SOINSPRRARSVASQS1 (SEQ ID NO: 31).

In an embodiment of the present invention are immunoactive polypeptides identified below and the corresponding cDNA sequences that are optimized for expression in mammalian cells, in particular Homo sapiens:

Peptide P3
(SEQ ID NO: 39)
MGGGGSQTNSPRRARSVASQSIGGGGSQTNSPRRARSVASQSIGGGGSQT
NSPRRARSVASQSI.
P3 Homo sapiens (Mammalian Cells) optimized cDNA
sequences
(SEQ ID NO: 40)
ATGGGCGGCGGCGGCAGCCAGACCAACAGCCCCAGGAGGGCCAGGAGCGT
GGCCAGCCAGAGCATCGGCGGCGGCGGCAGCCAGACCAACAGCCCCAGGA
GGGCCAGGAGCGTGGCCAGCCAGAGCATCGGCGGCGGCGGCAGCCAGACC
AACAGCCCCAGGAGGGCCAGGAGCGTGGCCAGCCAGAGCATC

See FIGS. 20C-20D for optimized cDNA sequences for E. coli K12 (SEQ ID NO: 41) and Saccharomyces Cerevisiae (SEQ ID NO: 42).

P3-L/AD
(SEQ ID NO: 47)
MASYQTQTNSPRRARSVASQSIIAYTMSLGAENGGGGSASYQTQTNSPRR
ARSVASQSIIAYTMSLGAENGGGSASYQTQTNSPRRARSVASQSIIAYTM
SLGAENGGGGSRTSYLLLFTLCLLLSEMASGGNFLTGLGHRSDHYNCVSS
GGQCLYSACPIFTKIQGTCYRGKAKCCK
P3-L/AD Homo sapiens (Mammalian Cells) cDNA
optimized sequence
(SEQ ID NO: 48)
ATGGCCAGCTACCAGACCCAGACCAACAGCCCCAGGAGGGCCAGGAGCGT
GGCCAGCCAGAGCATCATCGCCTACACCATGAGCCTGGGCGCCGAGAACG
GCGGCGGCGGCAGCGCCAGCTACCAGACCCAGACCAACAGCCCCAGGAGG
GCCAGGAGCGTGGCCAGCCAGAGCATCATCGCCTACACCATGAGCCTGGG
CGCCGAGAACGGCGGCGGCAGCGCCAGCTACCAGACCCAGACCAACAGCC
CCAGGAGGGCCAGGAGCGTGGCCAGCCAGAGCATCATCGCCTACACCATG
AGCCTGGGCGCCGAGAACGGCGGCGGCGGCAGCAGGACCAGCTACCTGCT
GCTGTTCACCCTGTGCCTGCTGCTGAGCGAGATGGCCAGCGGCGGCAACT
TCCTGACCGGCCTGGGCCACAGGAGCGACCACTACAACTGCGTGAGCAGC
GGCGGCCAGTGCCTGTACAGCGCCTGCCCCATCTTCACCAAGATCCAGGG
CACCTGCTACAGGGGCAAGGCCAAGTGCTGCAAG

See FIGS. 21C-21D for optimized cDNA sequences for E. coli (SEQ ID NO: 49) and Saccharomyces Cerevisiae (SEQ ID NO: 50).

SJ-80
(SEQ ID NO: 51)
MNVFQTRAGCLIGAEHVNNSYECDIPIGAGICASYQTQTNSPRRARSVA
SQSIIAYTMSLGAENSVAYSNN SIAIPTNFTI
SJ-80 Homo sapiens (Mammalian Cells) optimized
sequence
(SEQ ID NO: 52)
ATGAACGTGTTCCAGACCAGGGCCGGCTGCCTGATCGGCGCCGAGCACG
TGAACAACAGCTACGAGTGCGACATCCCCATCGGCGCCGGCATCTGCGC
CAGCTACCAGACCCAGACCAACAGCCCCAGGAGGGCCAGGAGCGTGGCC
AGCCAGAGCATCATCGCCTACACCATGAGCCTGGGCGCCGAGAACAGCG
TGGCCTACAGCAACAACAGCATCGCCATCCCCACCAACTTCACCATC

See FIGS. 22C-22D for optimized cDNA sequences for E. coli (SEQ ID NO: 53) and Saecharomyves Cereviseae (SEQ ID NO: 54).

SJ-100
(SEQ ID NO: 55)
MPVAIHADQLTPTWRVYSTGSNVFQTRAGCLIGAEHVNNSECDIPIGAG
ICASYQTQTNSPRRARSVASQ SIIAYTMSLGAENSVAYSNNSIAIPTN
FTI
SJ-100 Homo sapiens (Mammalian Cells) cDNA
optimized sequence
(SEQ ID NO: 56)
ATGCCCGTGGCCATCCACGCCGACCAGCTGACCCCCACCTGGAGGGTGT
ACAGCACCGGCAGCAACGTGTTCCAGACCAGGGCCGGCTGCCTGATCGG
CGCCGAGCACGTGAACAACAGCTACGAGTGCGACATCCCCATCGGCGCC
GGCATCTGCGCCAGCTACCAGACCCAGACCAACAGCCCCAGGAGGGCCA
GGAGCGTGGCCAGCCAGAGCATCATCGCCTACACCATGAGCCTGGGCGC
CGAGAACAGCGTGGCCTACAGCAACAACAGCATCGCCATCCCCACCAAC
TTCACCATC

See FIGS. 23C-23D for optimized cDNA sequences for E. coli K12 (SEQ ID NO: 57) and Saecheavinyces Cerevisiae (SEQ ID NO: 58).

SJ-120
(SEQ ID NO: 59)
MGTNTSNQVAVLYQDVNCTEVPVAIHADQLTPTWRVYSTGSNVFQTRAG
CLIGEHVNNSYECDIPIGAGICASYQTQTNSPRRARSVASQSIIAYTMS
LGAENSVAYSNNSIAIPTNFTI
SJ-120 Homo sapiens (Mammalian Cells) optimized
cDNA sequence
(SEQ ID NO: 60)
ATGGGCACCAACACCAGCAACCAGGTGGCCGTGCTGTACCAGGACGTGA
ACTGCACCGAGGTGCCCGTGGCCATCCACGCCGACCAGCTGACCCCCAC
CTGGAGGGTGTACAGCACCGGCAGCAACGTGTTCCAGACCAGGGCCGGC
TGCCTGATCGGCGCCGAGCACGTGAACAACAGCTACGAGTGCGACATCC
CCATCGGCGCCGGCATCTGCGCCAGCTACCAGACCCAGACCAACAGCCC
CAGGAGGGCCAGGAGCGTGGCCAGCCAGAGCATCATCGCCTACACCATG
AGCCTGGGCGCCGAGAACAGCGTGGCCTACAGCAACAACAGCATCGCCA
TCCCCACCAACTTCACCATC

See FIGS. 24C-24D for optimized eDNA sequences for E.coli K12 (SEQ ID NO: 61) ac Sacccharmyces Cerevisiae (SEQ ID NO: 62).

In an embodiment of the present invention is the immunonetive polypeptides identified below and the corresponding (DNA sequences, that are optimized for expression in mammalian cells, in particular Homo sapiens:

P3-L
(SEQ ID NO: 43)
MASYQTQTNSPRRARSVASQSIIAYTMSLGAENGGGGSASYQTQTNSPR
RARSVASQSIIAYTMSLGAENGGGGSASYQTQTNSPRRARSVASQSIIA
YTMSLGAEN
P3-L Homo sapiens (Mammalian Cells) cDNA
optimized sequence
(SEQ ID NO: 44)
ATGGCCAGCTACCAGACCCAGACCAACAGCCCCAGGAGGGCCAGGAGCG
TGGCCAGCCAGAGCATCATCGCCTACACCATGAGCCTGGGCGCCGAGAA
CGGCGGCGGCGGCAGCGCCAGCTACCAGACCCAGACCAACAGCCCCAGG
AGGGCCAGGAGCGTGGCCAGCCAGAGCATCATCGCCTACACCATGAGCC
TGGGCGCCGAGAACGGCGGCGGCGGCAGCGCCAGCTACCAGACCCAGAC
CAACAGCCCCAGGAGGGCCAGGAGCGTGGCCAGCCAGAGCATCATCGCC
TACACCATGAGCCTGGGCGCCGAGAAC

See FIGS. 20C-20D for optimized cDNA sequences for E. coli K12 (SEQ ID NO: 45) and Saccharomyces Cerevisiae (SEQ ID NO: 46)

In an enibothment of the present. invention is the immunoactive polypeptides identified below and the corresponding cDNA sequences that are optimized for expression in mammalian cells, in particular Homo saphiens:

SJ-FT
(SEQ ID NO: 144)
ECDIPIGAGICASYQTQTNSPRRARSVASQSIIAYTMSLGAENSVAYSNNSIAIP
TNFTISVTTEILPVSMTKTSVDCTMYICGDSTECSNLLLQYGSFCTQLNRALTGIAVEQ
DKNTQEVFAQVKQIYK TPPIKDFGGF NFSQILPDPS KPSKRSFIED LLFNKVTLAD
AGFIKQYGDCLGDIAARDLI CAQKFNGLTV LPPLLTDEMI
AQYTSALLAGTITSGWTFGA
SJ-FT Homo sapiens (Mammalian Cells) cDNA optimized sequence
(SEQ ID NO: 145)
GAGTGCGACATCCCCATCGGCGCCGGCATCTGCGCCAGCTACCAGACCCA
GACCAACAGCCCCAGGAGGGCCAGGAGCGTGGCCAGCCAGAGCATCATCGCCTA
CACCATGAGCCTGGGCGCCGAGAACAGCGTGGCCTACAGCAACAACAGCATCGC
CATCCCCACCAACTTCACCATCAGCGTGACCACCGAGATCCTGCCCGTGAGCATG
ACCAAGACCAGCGTGGACTGCACCATGTACATCTGCGGCGACAGCACCGAGTGC
AGCAACCTGCTGCTGCAGTACGGCAGCTTCTGCACCCAGCTGAACAGGGCCCTG
ACCGGCATCGCCGTGGAGCAGGACAAGAACACCCAGGAGGTGTTCGCCCAGGTG
AAGCAGATCTACAAGACCCCCCCCATCAAGGACTTCGGCGGCTTCAACTTCAGCC
AGATCCTGCCCGACCCCAGCAAGCCCAGCAAGAGGAGCTTCATCGAGGACCTGC
TGTTCAACAAGGTGACCCTGGCCGACGCCGGCTTCATCAAGCAGTACGGCGACT
GCCTGGGCGACATCGCCGCCAGGGACCTGATCTGCGCCCAGAAGTTCAACGGCC
TGACCGTGCTGCCCCCCCTGCTGACCGACGAGATGATCGCCCAGTACACCAGCGC
CCTGCTGGCCGGCACCATCACCAGCGGCTGGACCTTCGGCGCC

See FIGS. 25C-25D for optimized cDNA sequences for E. coli K12 (SEQ ID NO: 146) and Saccharomyces Cerevisiae (SEQ ID NO: 147).

SJ-S2′/FP1
(SEQ ID NO: 148)
TQLNRALTGIAVEQDKNTQEVFAQVKQIYKTPPIKDFGGFNFSQILPDP
SKPSKRSFIEDLLFNKVTLADAGFIKQYGDCLGDIAARDLICAQKFNGL
TVLPPLLTDEMIAQYTSALLAGTITSGWTFGAGAALQIPFAM
SJ-S2′/FP1 Homo sapiens (Mammalian Cells)
optimized cDNA sequences
(SEQ ID NO: 149)
ACCCAGCTGAACAGGGCCCTGACCGGCATCGCCGTGGAGCAGGACAAGA
ACACCCAGGAGGTGTTCGCCCAGGTGAAGCAGATCTACAAGACCCCCCC
CATCAAGGACTTCGGCGGCTTCAACTTCAGCCAGATCCTGCCCGACCCC
AGCAAGCCCAGCAAGAGGAGCTTCATCGAGGACCTGCTGTTCAACAAGG
TGACCCTGGCCGACGCCGGCTTCATCAAGCAGTACGGCGACTGCCTGGG
CGACATCGCCGCCAGGGACCTGATCTGCGCCCAGAAGTTCAACGGCCTG
ACCGTGCTGCCCCCCCTGCTGACCGACGAGATGATCGCCCAGTACACCA
GCGCCCTGCTGGCCGGCACCATCACCAGCGGCTGGACCTTCGGCGCCGG
CGCCGCCCTGCAGATCCCCTTCGCCATG

See FIGS. 26C-26D for optimized cDNA sequences for E. coli K12 (SEQ ID NO: 150) and Saccharomyces Cerevisiae (SEQ ID NO: 151).

SJ-S2′/FP1-2
(SEQ ID NO: 152)
SIIAYTMSLGAENSVAYSNNSIAIPTNFTISVTTEILPVSMTKTSVDCT
MYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGW
TFGAGAALQIPFAMQMAYRFNGIG
SJ-S2′/FP1-2 Homo sapiens (Mammalian Celis)
optimized cDNA sequences
(SEQ ID NO: 153)
AGCATCATCGCCTACACCATGAGCCTGGGCGCCGAGAACAGCGTGGCCT
ACAGCAACAACAGCATCGCCATCCCCACCAACTTCACCATCAGCGTGAC
CACCGAGATCCTGCCCGTGAGCATGACCAAGACCAGCGTGGACTGCACC
ATGTACATCTGCGGCGACAGCACCGAGTGCAGCAACCTGCTGCTGCAGT
ACGGCAGCTTCTGCACCCAGCTGAACAGGGCCCTGACCGGCATCGCCGT
GGAGCAGGACAAGAACACCCAGGAGGTGTTCGCCCAGGTGAAGCAGATC
TACAAGACCCCCCCCATCAAGGACTTCGGCGGCTTCAACTTCAGCCAGA
TCCTGCCCGACCCCAGCAAGCCCAGCAAGAGGAGCTTCATCGAGGACCT
GCTGTTCAACAAGGTGACCCTGGCCGACGCCGGCTTCATCAAGCAGTAC
GGCGACTGCCTGGGCGACATCGCCGCCAGGGACCTGATCTGCGCCCAGA
AGTTCAACGGCCTGACCGTGCTGCCCCCCCTGCTGACCGACGAGATGAT
CGCCCAGTACACCAGCGCCCTGCTGGCCGGCACCATCACCAGCGGCTGG
ACCTTCGGCGCCGGCGCCGCCCTGCAGATCCCCTTCGCCATGCAGATGG
CCTACAGGTTCAACGGCATCGGC

See FIGS. 27C-27D for optimized cDNA sequences for E.coli K12 (SEQ ID NO: 154) and Saccharomyces Cerevisiae (SEQ ID NO: 1.55),

SJ-FT/AD
(SEQ ID NO: 156)
ECDIPIGAGICASYQTQTNSPRRARSVASQSIIAYTMSLGAENSVAYSN
NSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFN
GLTVLPPLLTDEMIAQYTSALLAGTITSGWTFGAGGGGSGNFLTGLGHR
SDHYNCVSSGGQCLYSACPIFTKIQGTCYRGKAKCCK
SJ-FT/AD Homo sapiens (Mammalian Cells)
optimized cDNA sequences
(SEQ ID NO: 157)
GAGTGCGACATCCCCATCGGCGCCGGCATCTGCGCCAGCTACCAGACCC
AGACCAACAGCCCCAGGAGGGCCAGGAGCGTGGCCAGCCAGAGCATCAT
CGCCTACACCATGAGCCTGGGCGCCGAGAACAGCGTGGCCTACAGCAAC
AACAGCATCGCCATCCCCACCAACTTCACCATCAGCGTGACCACCGAGA
TCCTGCCCGTGAGCATGACCAAGACCAGCGTGGACTGCACCATGTACAT
CTGCGGCGACAGCACCGAGTGCAGCAACCTGCTGCTGCAGTACGGCAGC
TTCTGCACCCAGCTGAACAGGGCCCTGACCGGCATCGCCGTGGAGCAGG
ACAAGAACACCCAGGAGGTGTTCGCCCAGGTGAAGCAGATCTACAAGAC
CCCCCCCATCAAGGACTTCGGCGGCTTCAACTTCAGCCAGATCCTGCCC
GACCCCAGCAAGCCCAGCAAGAGGAGCTTCATCGAGGACCTGCTGTTCA
ACAAGGTGACCCTGGCCGACGCCGGCTTCATCAAGCAGTACGGCGACTG
CCTGGGCGACATCGCCGCCAGGGACCTGATCTGCGCCCAGAAGTTCAAC
GGCCTGACCGTGCTGCCCCCCCTGCTGACCGACGAGATGATCGCCCAGT
ACACCAGCGCCCTGCTGGCCGGCACCATCACCAGCGGCTGGACCTTCGG
CGCCGGCGGCGGCGGCAGCGGCAACTTCCTGACCGGCCTGGGCCACAGG
AGCGACCACTACAACTGCGTGAGCAGCGGCGGCCAGTGCCTGTACAGCG
CCTGCCCCATCTTCACCAAGATCCA

See FIGS. 28C-27D for optimized (DNA sequences for E. coli K12 (SEQ ID NO: 2158) and Saccharomyces Cerevisive (SEQ ID NO: 159),

SJ-S2′/FP1/AD
(SEQ ID NO: 160)
TQLNRALTGIAVEQDKNTQEVFAQVKQIYKTPPIKDFGGFNFSQILPDP
SKPSKRSFIEDLLFNKVTLADAGFIKQYGDCLGDIAARDLICAQKFNGL
TVLPPLLTDEMIAQYTSALLAGTITSGWTFGAGAALQIPFAMGGGGSGN
FLTGLGHRSDHYNCVSSGGQCLYSACPIFTKIQGTCYRGKAKCCK
SJ-S2′/FP1/AD Homo sapiens (Mammalian Cells)
optimized cDNA sequences
(SEQ ID NO: 161)
ACCCAGCTGAACAGGGCCCTGACCGGCATCGCCGTGGAGCAGGACAAGA
ACACCCAGGAGGTGTTCGCCCAGGTGAAGCAGATCTACAAGACCCCCCC
CATCAAGGACTTCGGCGGCTTCAACTTCAGCCAGATCCTGCCCGACCCC
AGCAAGCCCAGCAAGAGGAGCTTCATCGAGGACCTGCTGTTCAACAAGG
TGACCCTGGCCGACGCCGGCTTCATCAAGCAGTACGGCGACTGCCTGGG
CGACATCGCCGCCAGGGACCTGATCTGCGCCCAGAAGTTCAACGGCCTG
ACCGTGCTGCCCCCCCTGCTGACCGACGAGATGATCGCCCAGTACACCA
GCGCCCTGCTGGCCGGCACCATCACCAGCGGCTGGACCTTCGGCGCCGG
CGCCGCCCTGCAGATCCCCTTCGCCATGGGCGGCGGCGGCAGCGGCAAC
TTCCTGACCGGCCTGGGCCACAGGAGCGACCACTACAACTGCGTGAGCA
GCGGCGGCCAGTGCCTGTACAGCGCCTGCCCCATCTTCACCAAGATCCA
GGGCACCTGCTACAGGGGCAAGGCCAAGTGCTGCAAG

See FIGS. 29C-29D for optimized cDNA sequences for E. coli K12 (SEQ ID NO: 162) and Saccharomyces Cerevisiae (SEQ ID NO: 163).

SJ-S2′/FP1-2/AD
(SEQ ID NO: 164)
SIIAYTMSLGAENSVAYSNNSIAIPTNFTISVTTEILPVSMTKTSVDCT
MYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGW
TFGAGAALQIPFAMQMAYRFNGIGGGGGSGNFLTGLGHRSDHYNCVSSG
GQCLYSACPIFTKIQGTCYRGKAKCCK
SJ-S2′/FP1-2/AD Homo sapiens (Mammalian Cells)
optimized cDNA sequences
(SEQ ID NO: 165)
AGCATCATCGCCTACACCATGAGCCTGGGCGCCGAGAACAGCGTGGCCT
ACAGCAACAACAGCATCGCCATCCCCACCAACTTCACCATCAGCGTGAC
CACCGAGATCCTGCCCGTGAGCATGACCAAGACCAGCGTGGACTGCACC
ATGTACATCTGCGGCGACAGCACCGAGTGCAGCAACCTGCTGCTGCAGT
ACGGCAGCTTCTGCACCCAGCTGAACAGGGCCCTGACCGGCATCGCCGT
GGAGCAGGACAAGAACACCCAGGAGGTGTTCGCCCAGGTGAAGCAGATC
TACAAGACCCCCCCCATCAAGGACTTCGGCGGCTTCAACTTCAGCCAGA
TCCTGCCCGACCCCAGCAAGCCCAGCAAGAGGAGCTTCATCGAGGACCT
GCTGTTCAACAAGGTGACCCTGGCCGACGCCGGCTTCATCAAGCAGTAC
GGCGACTGCCTGGGCGACATCGCCGCCAGGGACCTGATCTGCGCCCAGA
AGTTCAACGGCCTGACCGTGCTGCCCCCCCTGCTGACCGACGAGATGAT
CGCCCAGTACACCAGCGCCCTGCTGGCCGGCACCATCACCAGCGGCTGG
ACCTTCGGCGCCGGCGCCGCCCTGCAGATCCCCTTCGCCATGCAGATGG
CCTACAGGTTCAACGGCATCGGCGGCGGCGGCGGCAGCGGCAACTTCCT
GACCGGCCTGGGCCACAGGAGCGACCACTACAACTGCGTGAGCAGCGGC
GGCCAGTGCCTGTACAGCGCCTGCCCCATCTTCACCAAGATCCAGGGCA
CCTGCTACAGGGGCAAGGCCAAGTGCTGCAAG

See FIGS. 30C and 30D for optimized cDNA sequences for E. coli (SEC) ID NO: 166) and Saccharomyces Cerevisiae (SEQ. ID NO: 167).

The present invention is not limited to the use of the foregoing peptides (or DNA or RNA encoding the same) individually or as mixtures of discrete peptides. indeed, it is within the scope of the present invention for one or more of the aminenic peptides to he covalendy linked to form a single-chain polypeptide. The single-chain polypeptide can be a recombinant or synthetic variant. The single-chain polypeptide further comprises a linker sequence. In some embodiments, the linker sequence can be a flexible linker sequence. In some embodiments, the linker sequence is a synthetic linker sequence, Although any linker may be used, it is preferred that the linker be of the (G₄S)n type wherein n is an integer of 1, 2, or 3, with 1 being most preferred. It is also possible to use a 218S linker.

Included amongst the antigenic peptides of the present invention are multi-epitope subunits defined herein above as P3-L P3-L/AD, SJ80, SJI00, and SJ120. Also included in the present invention is the adjuvanted ham (AD) of P3, SJ80, SJ100, and SJ120. The adjuvanted form has the human β1 defensin sequence added to the C-terminus of these multi-epitope subunits. This sequence appears as residues 108-178 of P3-L/AD (SEQ ID NO: 47).

Variants of these multi-epitope subunits haying at least 90% sequence identity, at least 92.5% sequence identity, at least 95% sequence identity, or at least 97.5% sequence identity are permitted so long as the immunogenicity is maintained.

Any mixture of the foregoing antigenic peptides is envisioned in the present invention. However, the following vaccine peptide mixtures are preferred:
Vaccine Peptides mix 1:

P22/v-P231v-P24-3epF25-PepF24-PepF23-PepF22-PepF21-PepF20-PepF19-PepF10-PepF7-PepF6-PepF5
Vaccine Peptides mix 2:

PepF7-PepF6-Pt,F5-P -B7lsv-PI 9-B8/sv-P19-117/sv-P22/v-P23/-v-PepF20-A26-B15

Although certain preferred or optimized nucleic acid sequences are specifically identified above, the present invention further entails DNA and RNA encoding each of the foregoing polypeptide sequences. The full scope of these sequences would be readily apparent to the skilled artisan. Moreover, the polypeptides and nucleic acids of the present invention may be purified, recombinant, or synthetic

Compositions and Vaccines. The invention also concerns a composition or vaccine comprising a SARS-CoV-2 nucleic acid, vector or peptide of the invention and, optionally, a suitable excipient and or adjuvant.

The peptides for use in the composition and vaccines of the present invention include one or more peptides as described above, as well as the Examples and the Figures. In some embodiments of the present invention, the peptides are used as a mixture of individual peptides or are covalently linked:

The excipient and adjuvants for use in the composition and vaccines of the present invention include those set forth above for which additional examples are provided below,

If desired the peptides may be admixed or coated with an intiminoadjuvant. Examples immunoadjuvants include aluminum (e.g., one or more of the following: amorphous aluminum hydroxyphosphate sulfate (AAHS), aluminum hydroxide, aluminum phosphate, potassium aluminum sulfate (Alum)), monophosphoryl lipid A (MPL)+aluminum salt (AS04), oil-in-water emulsion composed of squalene (MF59), monophosphoryl lipid A (MPL) and QS-21, a natural compound extracted from the Chilean soapbark tree, combined in a liposomal formulation (AS01_{B}), Cytosine phosphoguanine (CpG), a synthetic form of DNA that mimics bacterial and viral genetic material (e.g., CpG 1018), calcium phosphate, etc.

For the vaccines of the present invention, a number of SARS-CoV-2 antigenic peptides have been identified, which can be used in combination with each other in a peptide vaccine or in a nucleic acid vaccine (DNA or RNA).

In order to increase the immunogenicity of the composition, in some embodiments, the compositions/vaccine of the present invention preferably comprises one or more adjuvants and cytokines. In addition to the adjuvants and other materials that may be added to the compositions/vaccine of the present invention the following additional examples may be mentioned.

A suitable adjuvant based on route of administration may be selected by, one skilled in the art. Adjuvants which may be used with the attenuated virus, virus nucleic acids, or viral proteins include complete Freund's adjuvant (CFA), meomplete Freund's adjuvant (IFA), alumina adjuvants such as aluminum hydroxide or aluminum phosphate, microinanoparticles, antigen/peptide depot administration, immunopotentiators, adjuvants targeting toll-like receptors including TLR3, TLR 4, TLF7/8 and TLR9 agonists, STING agonists, cytokines such as IL-2, GM C: and interferons, liposomes, detergents such as Quil A or saponin, and bacterial products such as lipopolysaccharide, killed B. pertussis or mycobacterium, or toxoids, An adjuvant may also comprise an immunostimulatory Oligoflucleotide including ODN (DNA-based), ORN (RNA-based) oligonucleotides, or chimeric ODN-ORN structures, which may have modified backbones including, without phosphorothioate modifications, halogenations, alkylation (e.g ethyl- or methyl-modifications), and phosphodiester modifications. In some embodiments, poly inosinic-cytidylic acid or derivative thereof (poly LC) may be used,

These and other adjuvants are incorpor ated by reference to Khong, H. & Overwijk, W. W, Adjuvants for peptide-base cancer Vaccines, J.Immunotherapy of Cancer, 2016, 4, 56 and to those used by commercial adjuvant services, such as those described by Creative Biolabs hypertext transfer protocol secur://www.creative-biolabs.com/vaccine/vaccine-technology.htm (last accessed Nov. 9, 2021, incorpor ated by reference).

The amount of the added adjuvant or cytokine is not particularly limited but can generally be in an amount of about 0.01 lug to about 10 mg per dose, about 0:1 mg to about 5 mg, per dose, about 0.5 mg to about 2.5 mg per dose, or about 1 mg to 2 mg per dose, Alternatively, the adjuvant or cytokine may be at a concentration of about 0.01 to 50 wt %, 0.1 to 40 wt %, 1 to 30 wt %, or 2 to 25 wt %.

A particular composition or vaccine of the invention comprises a SARS-CoV-2 nucleic acid, vector or peptide as described herein and, optionally, a suitable excipient and/or adjuvant.

Typically, vaccines of the invention comprise an effective amount of at least one SARS-CoV-2 nucleic acid (including mRNA), vector or peptide. For example, the dosage volume ranges from 0.1 mL to 1 mL, preferably 0.25 mL to 0.75 mL, more preferably 0.3 to 0.7 mL, and most preferably 0.4 mL to 0.6 mL inclusive of the following exemplary dosage volumes 0.1 mL, 0.2 mL, 0.25 mL, 0.3 mL, 0.4 mL, 0.5 mL, 0.6 mL, 0.7 mL, 0.75 mL, 0.8 mL, 0.9 mL, and 1.0 mL. Further, nucleic acids (including mRNA), vectors or peptides are typically administered in the range of 1 pg to 1 mg, 10 pg to 500 μg, 100 pg to 250 μg, 500 pg to 150 μg, or 1 μg to 125 μg for particle mediated delivery and 1 μg to 1 mg, 1 μg to 500 μg, 5 μg to 250 μg, 10 μg to 125 μg, or 50 μg to 100 μg for other routes.

The vaccines and compositions according to the present invention may comprise any suitable excipient such as a solvent, diluent, carrier, stabilizer or the like. Examples of excipients include, for example, an aqueous buffer or a phosphate buffer, a physiological satine (0.85%), phosphate-buffered saline (PBS), citrate buffers, tris(hydroxymethyl anunomethane (TRIS), Tris-buffered saline and the like.

Preferably, the vaccines also comprise a further adjuvant or immunogenic carrier. Additional adjuvants may be obtained from any source including proteins and peptides derived from animals (e.g., hormones, cytokines, co-stimulatory factors), nucleic acids derived from viruses and other sources single-stranded RNA (e.g., mRNA) double-stranded RNA CpG), aluminum hydroxide, plant extracts, and the like, and are administered with the vaccine in an amount sufficient to enhance the immune response.

The vaccine of the present invention is preferably prepared under sterile conditions and/or sterilized after preparation in accordance with well-known techniques in the art.

As described herein, the vaccine of the present invention may be a nucleic acid vaccine (DNA or RNA vaccine).

Wherein the nucleic acid vaccine is a DNA vaccine, the DNA may encode the antigenic peptide in a plasmid/vector with a promoter and appropriate transcription and translation control elements, in some embodiments, the plasmid/vector may also include sequences to enhance, for example, expression levels, intracellular targeting, or proteasomal processing. In some embodiments, DNA vaccines comprise a viral vector containing a nucleic acid sequence encoding one or more polypeptides of the disclosure.

In additional aspects, the compositions disclosed herein comprise one or more nucleic Bio acids encoding peptides determined to have immunoreactivity with a biological sample. For example, in some embodiments, the compositions comprise one or more nucleotide sequences encoding peptides comprising a fragment that is a T cell epitope capable of binding to HLA class I molecules and/or HLA class II molecules of the subject. In some embodiments, the DNA vaccine also encodes inummomoduiatoty molecules to manipulate the resulting immune responses, such as enhancing the potency of the vaccine, stimulating the immune system or reducing immunosuppression. Strategies for enhancing the immunogenicity of DNA vaccines include encoding of xenogeneic versions of antigens, fusion of antigens to molecules that activate T cells or trigger associative recognition, priming with DNA vectors followed by boosting with viral vector, and utilization of immunomodulatory molecules.

In some forms the DNA vaccine is incorpor ated into liposomes or other forms of nanobodies. In some embodiments, the DNA vaccine includes a delivery system selected from the group consisting of a transfection agent; protamine; a protamine liposome; a polysaccharide particle; a cationic nanoemulsion; a cationic polymer; a cationic polymer liposome; a cationic nanoparticle; a cationic lipid and cholesterol nanoparticle; a cationic lipid, cholesterol, and PEG nanoparticle; a dendrimer nartoparticle.

Wherein the nucleic acid vaccine is an RNA vaccine, the RNA is non-replicating mRNA or virally derived, self-amplifying RNA. In some embodiments, the non-replicating mRNA encodes the peptides disclosed herein and contains 5′ and 3′ untranslated regions (UTRs). In some embodiments, the virally derived, self-amplifying RNA encodes nut only the peptides disclosed herein but also the viral replication machinery that enables intracellular RNA amplification and abundant protein expression,

In some embodiments, the RNA is directly introduced into the subject. In some embodiments, the RNA is chemically synthesized or transcribed in vitro. In some embodiments, the mRNA is produced from a linear DNA template using a T7, a T3, or a Sp6 phage RNA polymerase, and the resulting product contains an open reading frame that encodes the peptides disclosed herein, flanking UTRs, as 5′ cap, and a poly(k) tail. In Some embodiments, various versions of 5′ caps are added during or after the transcription reaction using a vaccinia virus capping enzyme or by incorpor ating synthetic cap or anti-reverse cap analogues. In some embodiments, an optimal length of the poly(A) tail is added to mRNA either directly from the encoding DNA template or by using poly(A) polymerase. The RNA may encode one or more -peptides comprising a fragment that is a T cell epitope capable of binding to HLA class I and/or HLA class II molecules of a patient. In some entbodiments, the RNA includes signals to enhance stability and translation, in some embodiments, the RNA also includes unnatural nucleotides, to increase the half-life or modified nucleosides to change the immunostimulatory profile.

In some embodiments, the RNA vaccine is incorpor ated into liposomes o.r other forms of nanobodies that facilitate cellular uptake of RNA and. protect it from degradation. in some embodiments, the RNA vaccine includes a delivery system selected from the group consisting of a transfection agent; protamine; a protamine hposome; a polysaccharide particle; a cationic nanoemulsion; a cationic polymer; a eat iOnie polymer liposorne; a cationic nanoparticle; a cationic lipid and. cholesterol nanopartiele; a cationic lipid, cholesterol, and PEG nanoparticle; a dendrimer nanoparticle; and/or naked mRNA; naked mRNA with in vivo electropor ation; protamine-complexed mRNA; mRNA associated with a positively charged oil-in-water cationic minoemuision; raRNA associated with a chemically modified dendrimer and complexed with polyethylene glycol (PEG)-lipid; protamine-complexed mRNA in a PEG-lipid nanoparticie; mRNA associated with a cationic polymer such as polyethylenimine (PEI); mRNA associated with a cationic polymer such as PEI and a lipid component mRNA associated with a polysaccharide (for example, chitosan) particle or gel; mRNA in a cationic lipid nanopaincle (for example, 1,2 dioleoyloxy 3 trimethylammoniumpropane (DOTAP) or dioleoylphosphatidylethanolamine (DOPE) lipids); mRNA complexed with cationic lipids and cholesterol; or mRNA complexed with cationic lipids, cholesterol and PEG-lipid.

Uptake of polynuelccitide or ollgonucleotide constructs may be enhanced by several known transfection techniques, for example those including the use of transfection agents. Examples of these agents include cationic: agents, for example, calcium phosphate and DEAE-Dextran and lipofectants, for example, lipofeetam. and transfix:tam. The dosage of the polynucleotide or oligonucleotide to he administered can be altered.

As opposed to a vaccine that provides an active immunization against SARS-CoV-2 infection andfor amelior ates symptoms associated therewith, the composition of the present invention need not result in immunization. instead, the composition of the present invention. may be a pharmaceutical immunogenic composition useful for preventing or curing a coronavirus SARS-CoV-2 infection. To this end, the dosage of the active peptide in the composition of the present invention can be controlled such that it brings about the desired effect including stimulating an immune response. Further, the composition of the present invention may be used M combination with other SARS-CoV-2 treatment methods and/Or administered to a subject that is already positive for SARS-CoV-2 infection.

The Compositions or vaccines of the invention may further comprise or may be used in combination with other vaccines or antigens, for example an influenza vaccine;

The compositions or vaccines can be stored under appropriate conditions to preserve at least 75%, at least 80%, rat. least 85% at least 90%, at least 91%, at least 92%, at least 93%, at least 94%, at least 95%, at least 96%, at least 97%, at least 98%, at least 99%; or 100% of the immimogenicity of the composition or vaccine relative to the immunogenicity upon initial preparation. This can be achieved, for example, by adding preservatives or other components that are safe For administration to the subject (e.g., human), techniques such as lyophilization or by stor age with freezing with our without protection from light. With respect to stor age form, it is preferred that the 4-10 dosages be stored in each stor age vial to ensure that the composition or vaccine can be reconstituted or thawed immediately before use. Generally, the composition or vaccine should be stored frozen at between −50° to −15° C. (−58° to 5° F.). Once thawed, the composition or vaccine should not be refrozen and should be stored between 2° to 8° C. (36° to 46° F.) for up to 30 days:

Methods of Stimulating an Immune Response. The compositions or vaccines of the present invention as defined above can be used to stimulate an immune response against a SARS-Cov2 infection in, a subject. In this method, administered. to the subject (e.g., a human) is a composition or vaccine that contains an effective amount to stimulate the subject to produce antibodies to the antigenic peptide(s) contained therein or encoded by the nucleic acids in said composition or vaccine.

Vaccination Method, The SARS-CoV-2 vaccines of the present invention may be used to induce protective immunity m subjects, particularly humans, or to treat SARS-CoV-2-infected subjects. The invention is particularly suited to vaccinate subjects, in particular humans, to SARS-CoV-2 infection, in a preventive or prophylactic setting

The invention thus also relates to a SARS-CoV-2 vaccine as defined above, for use to immunize or vaccinate subjects (e.g., humans).

The invention relates to a method for inducing a SARS-CoV-2 immunity in a subject (e.g., human) comprising administering to said subject an SARS-CoV-2 vaccine as disclosed herein.

The methods of administration and dosage forms for the vaccine of the present invention are as described herein above.

In a preferred embodiment, the vaccination method is repeated two or more times where the subsequent doses are the same or different effective amounts.

In an embodiment of the present invention, following the administration of a first dose of the vaccine of the present invention a second dose of the vaccine is administered after a period of time has elapsed. Exemplary time periods between the first and second dose range from 3 days to 12 weeks, from 1 week to 10 weeks, from 2 weeks to 8 weeks, from 3 weeks to 6 weeks, from 4 weeks to 5 weeks, inclusive of 3 days, 1 week, 2 weeks, 3 weeks, 4 weeks, 5 weeks, 6 weeks, 7 weeks, 8 weeks, 9 weeks, 10 weeks, 11 weeks, and 12 weeks with the understanding that these intervals include 2-3 days on either side of the define number of weeks: The same time periods may be observed for third and subsequent doses.

In an embodiment or the present invention, the vaccination method (whether single dose administration or multi-dose administration) includes administration of a subsequent booster dosage. The booster dosage may be at the same or a different effective amount, Preferably, the effective amount of the booster dosage is less than the previously administered dosages. For example, in the case of a single dose administration or two-dose administration, the booster dosage is 75% or less, 67% or less, or 50% or less of the effective amount of the previous effective amount. The booster dosage may be administered 4 months to 4 years, 5 months to 3 years, 6 months to 2 years, or 9 months to 1 year after the completion of the primary vaccination method.

Composition of Purified Antibodies. In an embodiment of the present invention is a composition comprising antibodies that specifically bind to one or more antigenic peptides described herein, The antibodies are Obtained by (1) administering to a subject an effective amount of a composition or vaccine of the present invention, (2) allowing the subject to generate antibodies to the antigenic peptides contained in or produced in vivo by the composition or vaccine, (3) obtaining a blood or serum sample from the subject, and (4) isolating or purifying the antibodies from the blood or serum. Methods of purifying antibodies from blood or serum are well known in the art. The isolated or purified antibodies can be mixed with a pharmaceutically acceptable carrier, diluent, excipient or adjuvant or as defined herein to form a neutralizing antibody composition. The neutralizing antibody compositions are formulated .for different routes of administration (e.g, intravenous, subcutaneous) and to achieve the desired effect. Further, the antibodies can be liposomally packaged for delivery to prevent undesired degradation.

The isolated or purified antibodies should be validated for reaction with one or more of antigenic peptide of the present invention. The isolated or purified neutralizing antibodies can be subsequently used to inhibit SARS-CoV-2 virus multiplication in a subject in need thereof.

In some embodiments, the antibodies wherein may be used for treating a subject in need thereof. In some embodiments, a neutralizing antibody composition can be administered to a subject diagnosed to be infected with SARS-CoV-2.

Single or multiple administrations of neutralizing antibody compositions can be given to a subject in need thereof depending on for example: the dosage and frequency as required and tolerated by the subject. The formulation should provide a sufficient quantity of active agent to effectively treat, prevent, or amelior ate conditions, diseases, or symptoms associated with SARS-CoV-2.

Serlogical Diagnostic Assay.

The present invention provides a serological diaimostic assay for the detection of specific anti-SARS-CoV-2 IgG antibodies in human serum sample by detecting the presence of neutralizing antibodies against SARS-CoV-2 virus. This method entails:

A method for determining whether a subiect is positive for SARS-CoV-2 vats infection comprising:

a) preparing a serum, from said subject:;

b) contacting the serum with one or more purified antigenic peptide, polypeptide, or immunologically ftinctional analogue as defined herein;

c) detecting an immunologic reaction by adding labelled anti-human antibodies;

d) comparing the result of step (c) with a standard reference curve; and

e) classifying the subject as being positive for SARS-CoV-2 virus infection if the presence of SARS CoV-2 antibodies is detected in said serum.

In this method, prior to (a) is a step of obtaining a blood sample from a human subject:

The subject for the present method is a human that has been exposed to the SARS-CoV-2 virus, a human that has mild symptoms consistent with S— RS-infection, a human that has moderate symptoms consistent with SARS-CoV-2 infection, or a human that as severe symptoms consistent with SRS-COV-2 infection.

The antiaenic peptide, polypeptide, or immunologically functional analogue for use in the present method may be any as defined herein. The antigenic peptide, polypeptide, or immunologically functional analogue may be used individually or in a mixture. M an embodiment of the present invention the step (b) uses one or more of the antigenic peptides of SEQ ID NOs; 1-39, 43, 47, 51, 55, 59, 64-74, 108, 110-120, 144, 148, 152, 156, 160, and 164. In an embodiment of the present invention the step (b) uses one or more of the antigenic peptides of SEQ NOs: 1-30. In an embodiment of the present invention the step (b) uses one or more the antigenic peptides of SEQ ID NOs: 1-15. In an embodiment of the present invention the step (b) uses one or more of the antigenic peptides of SEQ ID NOs: 1-10 in an embodiment of the present invention the step (b) uses one or more of the antigenic peptides of SEQ ID NOs: 39, 43, 47, 51, 59, 116, 144, 148, 152, 156, 160, and 164. In an embodiment of the present invention, step (b) uses each of the antigenic peptides of SEQ ID NOs: 1-10. In an embodiment of the present invention, step (b) uses the antigenic peptide of SEQ ID NO: 3 alone or in combination with one or more other antigenic peptides described herein.

In an embodiment of the present invention, the antigenic peptides are synthetic or recombinant. In an embodiment of the present invention, the antigenic peptides are synthetic and have an N-terminal biotin group.

in an embodiment of this method of the present invention, said contacting is in a multi-well plate. In an embodiment of the present invention, the multi-well plate is a 96-well enzyme-linked immunosorbent assay (ELISA) plate. In an embodiment of the present invention, each well of the mild-well plate is coated with streptavidin, in an embodiment of the present invention, the streptavidin coated wells are blocked with non-fat milk powder and the plate is dried.

In an embodiment of the present invention, said contacting is by adding serum to streptavidin coated wells. In this embodiment, the serum samples are diluted, preferably 1:100 in a dilution buffer. in this embodiment, the serum samples are incubated in the streptavidin coated wells. In (his embodiment, the incubation is preferably for 1 hour at room temperature.

In an embodiment of they present invention, following said contacting the serum is removed from the streptavidin coated wells and the wells are washed.

In an embodiment of the present invention, the labelled anti-human antibodies are horse radish peroxidase (HRP)-conjugated goat anti-human IgG antibodies. In this embodiment, HRP-conjugated goat anti-human IgG antibodies are incubated in the wells.

In this embodiment, the incubation is preferably for 1 hour at room temperature. In this embodiment, following incubation the HRP-conjugated goat anti human IgG antibodies are removed by washing the wells. In this embodiment, following washing, horse radish peroxidase (HRP) substrate is added to each well. In this embodiment, the detection reaction is quenched by adding a H₂SO₄ solution. The plates are then analyzed by determining the absorbance (i.e., optical density) at 450 rim wavelength.

In an embodiment of the present invention, a standard reference curve is prepared representing SARS-CoV-2 virus negative. To establish the SARS-CoV-2 virus negative standard reference curve, (Cut off value) an OD 450 nm curve is prepared with historical sera obtained from healthy subjects long before the SARS-CoV-2 virus emerged. Optical density values [OD] at 450 nm ate transformed from numeric (float) type to factor type (categorical variable) using the following rule (The OD value threshold): OD<0.45 immune (−) and OD>0.45=Immune (+), for the epitopes displayed by peptide P3. A subject is defined as IgG positive (i.e., positive for SARS-CoV-2 virus infection) when the OD value is 2 standard deviations (SD) above the mean of the negative controls (n=100).

Method of Tracking anti SARS-CoV-2 Protective Immunity, The serological assay defined above showed that COVID19 recovered patients who had the asymptomatic or mild form of the disease developed high levels of specific antibodies to P3 (SEQ ID NO: 3), P3L (SEQ ID 43), P4/TMP-Pol3 (SEQ ID NO: 116), and/or SJ/FT (SEQ ID NO: 144) a protective immunity (i.e., a very high association between antibody response and disease asymptomatic/Mild forms). Thus, the use of the foregoing serological assay with peptide P3 (SEQ ID NO: 3) P3L, (SEQ ID 433), P4/TMP-Pol3 (SEQ ID NO: 116) and/or SJ/FT (SEQ ID NO:144) can reveal whether a human suspected to be infected or to have been infected by a SARS-COV-2 virus has generated an anti SARS-CoV-2 protective immunity. Further, the serological assay of the present invention can be used to track protective immunity from COVID19/SARS-CoV-2 infection by ascertaining whether a subject has generated levels of a specific antibodies for peptide P3 (SEQ ID 3), P3L (SEQ ID 43), P4/TMP-Pol3 (SEQ ID 116) and/or SPFT (SEQ ID 144) that exceed an OD value of 2 standard deviations (SD) above the mean of the negative controls (n=100). These peptides may

Kits. Also provided herein are kits that include any of the antigenic peptides, nucleic acids, compositions, vaccines or antibodies described herein. In some embodiments, the kits can include instructions for performing any of the methods described herein, in some embodiments, the kits can include at least one dose of any of the compositions or vaccines described herein. The present invention also provides a kit for detecting the presence of or for quantifying the antibodies recognizing SARS-CoV-2 in a human serum suspected to have been infected with SARS-CoV-2, or those in a vaccinated patient. The kit of this embodiment comprises controls consisting of positive and negative reagents and a set of reagents or active products comprising one or more of the antigenic peptides described herein in any of a purified, recombinant, or synthetic form whether a polypeptide, peptide, nucleic acid (-DNA or RNA) encoding the antigenic peptide, or antigenically or immunologically functional analogues.

Vaccination is important in combatting the COVID19 pandemic and. for saving human lives, However, despite an overall good efficiency, the currently existing vaccines have a number of pitfalls and might not be enough to combat efficiently SARS-Cov-2 virus infection. Through the identification of a SARS-COV-2 epitopes that elicit ptotective immunity to humans, this invention provides an opportunity for the rapid development of an alternative safe and efficient precision-vaccine against all SARS-CoV-2 virus genetic variants. It provides also a serological tool for studying and tracking protective immunity against SARS-CoV-2 infection.

The present invention as described herein and supported by the Examples below can be better understood by reference to the following illustrative and exemplary description of embodiments.

### Description of Exemplary Embodimenis

(1) An immunogenic peptide or nucleic acid encoding the same selected from the group consisting of SEQ ID NOs: 1-39, 43, 47, 51, 55, 59, 64-74, 108, 110.120, 144, 148, 152, 156, 160, and 164.

(2) The immunogenic peptide or nucleic acid of (1), which is an immunogenic peptide.

(3) The immunogenic peptide or nucleic acid of (2), wherein the immunogenic peptide has an N-terminal biotin group.

(4) The immunogenic peptide or nucleic acid of any of (1) to (3), wherein the immunogenic peptide is selected from the group consisting of SEQ ID NOs: 1-30, 64-74, and 110-120.

(5) The immunogenic peptide or nucleic acid of any of (1) to (4), wherein the immunogenic peptide is selected from the group consisting of SEQ ID NOs; 1-15.

(6) The immunogenic peptide or nucleic acid of any of (1) to (5), wherein the immunogenic peptide is selected from the group consisting of SEQ ID NOs: 1-10 and 110-120.

(7) The immunogenic peptide or nucleic acid of any of (1) to (6), wherein the immunogenic peptide is SEQ NO: 3

(8) The immunogenic peptide or nucleic acid of any of (1) to (3), wherein the immunogenic peptide is selected from the group consisting of SEQ ID NOs: 39, 43, 47, 51, 55, 59, 144, 148, 152, 156, 160, and 164;

(9) The immunogenic peptide or nucleic acid of any of (1) to (3), wherein the immunogenic peptide is one or more immunogenic peptides selected from the group consisting of SEQ ID NOs.; 1-7, 9-12, 14-15, 18, 31-38, and 108.

(10) The immunogenic peptide or nucleic acid of any of (1) to (3), wherein the immunogenic peptide is one or more immunogenic peptides selected from the group consisting of 8 and 13.

(11) The immunogenic peptide or nucleic acid of any of (1) to (10), wherein the immunogenic peptide is recombinant

(12) The immunogenic peptide or nucleic acid of any of (1) to (11), wherein the immunogenic peptide is synthetic.

(13) The immunogenic peptide or nucleic acid of any of (1) to (11), wherein the immunogenic peptide is purified.

(14) The immunogenic peptide or nucleic acid of any of (1) to (13), which is a in nucleic acid encoding said immunogenic peptide.

(15) The immunogenic peptide or nucleic. acid of (14, wherein the nucleic acid is DNA.

(16) The immunogenic peptide or nucleic acid of (14), wherein the nucleic acid is RNA.

(17) The immunogenic peptide or nucleic acid of (16), wherein the nucleic acid is mRNA.

(18) A set of immunogenic peptides comprising one or more immunogenic peptides selected from the group consisting of SEQ ID NOs: 1-39, 43, 47, 51, 55, 59, 64-74, 108, 110-120, 144, 148, 152, 156, 160, and 164.

(19) The set of (18), wherein the one or more immunoeenic peptides have an N-terminal biotin group.

(20) The set of any of (18) to (19), wherein the immunogenic peptide is one or more immunogenic peptides selected from the group consisting of SEQ ID NOs: 1-30, 64-74, and 110-120.

(21) The set of any of (18) to (20), wherein the immunogenic peptide is one more immunogenic peptides selected from the group consisting of SEQ ID NOs: 1-15.

(22) The set of any of (18) to (21), wherein the immunogenic peptide is one or more immunogenic peptides selected from the group consisting of SEQ ID NOs: 1-10 and 110-120.

(23) The set of any of (18) to (22), wherein the immunogenic peptide is at least the immunogenic peptide of SEQ ID NO: 3.

(24) The set of any of (18) to (19), wherein the immunogenic peptide is one or more immunogenic peptides selected from the group consisting of SEQ ID NOs: 1-7, 9-12, 14-15, 18, 31-38, and 108.

(25) The set of any of (18) to (19), wherein the immunogenic peptide is one or more immunogenic peptides selected from the group consisting of 8 and 13.

(26) The set of any of (18) to (19), wherein the immunogenic peptide is one or more immunogenic peptides selected from the group consisting of SEQ ID NOs: 39, 39, 43, 47, 51, 55, 59, 144, 148 152, 156, 160, and 164.

(27) The set of any of (18) to (26), wherein the immunogenic peptide is recombinant.

(28) The set of any of (18) to (26), wherein the immunogenic peptide is synthetic.

(29) The set of any of (18) to (28), wherein the immunogenic peptide is purified.

(30) A set of polynucleotides encoding the set of immunogenic peptides of any of (18) to (29).

(31) The set of polynucleotides of (30), wherein said polynucleotides are DNA.

(32) The set of polynucleotides of (30), wherein. said polynucleotides are RNA.

(33) The set of polynucleotides of (30), wherein the nucleic acid is mRNA.

(34) A composition comprising one or more immunogenic peptides or nucleic acid encoding the same as set forth in any of (1) to (17) and a pharmaceutically acceptable carrier, excipient and or adjuvant.

(35) A composition comprising set of immunogenic peptides as set forth in any of (18) to (28) or set of polynucleotides set forth in any of (30) to (33) and a pharmaceutically acceptable carrier, excipient and/or adjuvant.

(36) The composition as set forth in (34) or (35), wherein said composition contains an effective amount of the immunogenic peptide(s) or nucleic acid(s) encoding the same to induce synthesis of neutralizing antibodies inhibiting SARS-CoV-2 virus multiplication,

(37) A COVID19 vaccine comprising an effective amount of one or more immunogenic peptides or nucleic acid encoding the same as set forth in any of (1) to (17) and a pharmaceutically acceptable carrier, excipient and/or adjuvant.

(38) A COVID19 vaccine comprising an effective amount of the set of immunogenic peptides as set forth in any of (18) to (28) or set of polynucleotides as set forth in any of (30) to (33) and a pharmaceutically acceptable carrier, excipient and/or adjuvant,

(39) The COVID19 vaccine of (37) or (38), wherein said vaccine is a peptide vaccine.

(40) The COVID.I9 vaccine of (37) or (38), wherein said vaccine is a DNA vaccine.

(41) The COVID19 vaccine of (37) or (38), wherein said vaccine is an RNA vaccine.

(42) The COVID19 vaccine of (37) or (38), wherein said vaccine comprises one or more adjuvants and/or cytokines,

(43) The COVID19 vaccine of any of (37) to (42), wherein said effective amount. ranges from 1 pg to 1 mg.

(44) The COVID19 vaccine of any of (37) to (43), wherein the active component is incorpor ated into liposomes or other forms of nanobody.

(45) A method of inducing a protective immunity against SARS-CoV-2-infection in a subject in need thereof comprising administering to said subject a COVID19 vaccine of any of (37) to (44).

(46) The method of (45), wherein the said vaccine is administered in a single dose.

(47) The method of (45), wherein the said vaccine is administered in two or more doses.

(48) A composition of purified antibodies against immunogenic peptides as set forth in any of (1) to (17).

(49) A method for determining whether a subject is positive for SARS-CoV-2 virus infection comprising:

a) preparing a serum from said subject;

b) contacting the serum with one or more immunogenic peptides or nucleic acid encoding the same as set forth in any of (1) to (17), the set of immunogenic peptides as set forth in any of (18) to (28) or the set of polynucleotides as set forth in any of (30) to (33);

c) detecting an immunologic reaction by adding labelled anti-human antibodies;

d) comparing the result of step (c) with a standard reference curve: and

e) classifying the subject as being positive for SARS-CoV-2 virus infection if the presence of SARS CoV-2 antibodies is detected in said serum.

(50) The method of (49), wherein prior to (a) is a step of obtaining a blood sample from a human subject:

(51) The method of (49) or (50), wherein the subject is a human that has been exposed to the SARS-COV-2 virus.

(52) The method of any of (49) to (51), wherein the subject is a human that has mild symptoms consistent with SARS-CoV-2 infection.

(53) The method of any of (49) to (51), wherein the subject is a human that has moderate symptoms consistent with SARS-(,os'2 infection.

(54) The method of any of (49) to (51), wherein the subject is a human that has severe symptoms consistent with. SARS-CoV-2 infection.

(55) The method of any of (49) to (54), wherein the immunogenic peptide is one or more selected from the group consisting of SEQ ID NOs: 1-39, 43, 47, 51, 55, 59, 64-74, 108, 110-120, 144, 148, 152, 156, 160, and 164.

(56) The method of any of (49) to (55), wherein the immunogenic peptide has an N-terminal biotin group.

(57) The method of any of (49) to (56), wherein the immunogenic peptide is selected from the group consisting of SEQ ID NOs: 1-30, 64-74, and 110-120.

(58) The method of any of (49) to (56), wherein the immunogenic peptide is selected from the group consisting of SEQ ID NOs: 1-15.

(59) The method of any of (49) to (56), wherein the immunogenic: peptide is selected from the group consisting of SEQ NOs: 1-10 and 110-120.

(60) The method of any of (49) to (56), wherein the immunogenic peptide is SEQ ID NO; 3.

(61) The method of any of (49) to (56), wherein the immunogenic peptide is selected from the group consisting of SEQ ID NOs: 39, 43, 47, 1.51, 55, 59, 144, 148, 152, 156, 160, aid 164.

(62) The method of any of (49) to (61), wherein the immunogenic peptide is recombinant.

(63) The method of any of (49) to (61), wherein the immunogenic peptide is synthetic.

(64) The method of any of (49) to (43), wherein the immunogenic peptide is purified.

(65) The method of any of (49) to (64), wherein said contacting is in a multi-well plate.

(66) The method of (65), wherein each well of the multi-well plate is coated with streptavidin.

(67) The method of (66), wherein said contacting: is by adding serum to streptavidin coated wells.

(68) The method of (67), wherein said serum is diluted 1:100.

(69) The method of any of (49) to (68), wherein said labelled anti-human antibodies are horse radish peroxidase (HRP)-conjugated goat anti-human IgG antibodies and the detectable signal is the result of a reaction between the HRP-conjugated goat anti-human IgG antibodies and horse radish peroxidase (HRP) substrate.

(70) The method of (69), wherein detecting is by analyzing the optical density at a wavelength of 450 nm.

(71) The method of any of (49) to (70), whereina subject is classified is being positive for SARS-CoV-2 virus infection when the OD value at 450 nm is 3 standard deviations (SD) above the mean of the negative controls.

(72) A method of tracking protective immunity from COVID19/SARS-Co infection by ascertaining whether a subject has generated levels of a specific antibodies for peptide P3 (SEQ ID NO: 3) that exceed an OD value at 450 nm of 3 standard deviations (SD) above, the mean of the negative controls (n=100) by a method of any of (49) to (71).

(73) A kit for detecting the presence of or for quantifying the antibodies of a human serum suspected to have been infected or a vaccinated patient, said kit comprising positive and negative control reagents and one or more immunogenic peptides or nucleic acid encoding the same as set forth in any of (1) to (17), the set of immunogenic peptides as set forth in any of (18) to (7) or the set of polynucleotides as set fourth in any of (30) to (33).

(74) A synthetic or purified immunopeptide P3 (SEQ ID NO: 3).

(75) An immunogenic protective composition comprising immunopeptide P3 (SEQ ID NO: 3)

(76) A composition comprising, as a component, purified antibodies protecting a subject against a SARS CoV-2 infection said purified antibodies forming an immunocomplex with at least one immunogenic peptide of (1)-(17).

### Complementary Embodiments

(77) A polypeptide antigen comprising a SARS-CoV-2 S protein S1/S2 cut site or a S1/S2 cut site, including the P3 peptide (SEQ ID NO: 3), a variant thereof, or a chemically modified form thereof.

78) The polypeptide antigen of embodiment 77 that further comprises an N-terminal biotin group.

(79) The polypeptide antigen of embodiment further comprising a furin cut site or a Transmembrane Protease Serine 2 (INTPRSS2) cut site.

(80) The polypeptide antigen of embodiment 77 comprising a peptide selected from the group consisting of P3 (SEQ ID NO: 3), P3 L (SEQ ID NO: 43), SJ-FT (SEQ ID NO: 144) and. P4ITNIP-Pol3 (SEQ., ID NO: 116), SEQ ID NOs: 1, 2, 4-39, 47, 51.55, 59, 64-74, 108, 110-115, 117-120, 148, 152, 156, 160, and 164; or a chemically modified form thereof.

(81) The polypeptide antigen of embodiment 77 comprising a peptide selected from the group consisting of SEQ ID NOs: 3, 3L , 116 and 144 or a variant thereof or a recombinant or an engineered or chemically modified form thereof.

(82) The polypeptide antigen of embodiment 77 that comprises a peptide selected IU from the group consisting of SEQ ID NOs: 1-30, 64-74, and 110-120.

(83) Thepolypeptide antigen embodiment 77, wherein the peptide is selected from the group consisting of SEQ. ID NOs: 1-15.

(84) The polypeptide antigen of embodiment 77, wherein the peptide is selected from the group consisting of SEQ ID NOs: 1-10 and 110-120.

(85) The polypeptide antigen of embodiment 77, wherein the peptide is SEQ ID NO: 3.

(86) The polypeptide antigen of embodiment 77, wherein the peptide is selected from the group consisting of SEQ ID NOs: 39, 43.47, 51, 55, 59, 144, 148, 152, 156, 160, and 164.

(87) The polypeptide antigen of embodiment 77, wherein the peptide is one or more peptides selected from the group consisting, of SEQ ID NOs: 1-79-12, 14-15, 18, 31-38, and 108.

(88) The polypeptide antigen of embodiment 77, wherein the peptide is one or more peptides selected from the group consisting of SEQ ID NO: 8 and SEQ ID NO: 13.

(89) The polypeptide antigen of embodiment 77 that is a wild-type peptide that comprises 6 to 20 contiguous amino acid residue of a SARS-CoV-2 antigen.

(90) The polypeptide antigen of embodiment 77 that is a chimeric antigen or peptide or a peptide or antigen conjugate comprising the at least one peptide or antigen of embodiment 1.

(91) The polypeptide antigen of embodiment 77 that has been chemically modified to comprise an N-terminal biotin group, or at least one other exogenous chemical group or chemical modification.

(92) A composition comprising the poly-peptide antigen of embodiment or an engineered or chemically modified form thereof or a chemically modified form thereof and a carrier, excipient, and/or adjuvant.

(93) The composition of embodiment 92 that comprises two or more of said, polypeptide antigens.

(94) The composition of embodiment 92 that is immunogenic and which induces humoral or cellular immunity against SARS-CoV-2 when administered to a subject.

(95) A method for preventing or reducing the severity of an infection by SARS-CoV-2 comprising administering the-polypeptide antigen of embodiment 77, or a chemically modified form thereof, to a subject in need thereof.

(96) A method for detecting antibodies to SARS-COV-2 in a biological sample comprising contacting the sample with at least one antigen or a peptide, chimeric antigen or peptide, or peptide conjugate of embodiment 77, or a chemically modified thereof.

(97) A nucleic acid encoding at least one polypeptide antigen of embodiment 77 or a peptide, a chimeric antigen or a peptide, or an antigen or a polypeptide antigen conjugate or an engineered or chemically modified form thereof.

(98) A composition comprising the nucleic acid of embodiment 97 and a pharmaceutically acceptable carrier, excipient and/or adjuvant.

(99) A method for preventing or reducing the severity of an infection by SARS-CoV-2 comprising administering to a subject in need thereof a nucleic acid encoding the polypeptide antigen of embodiment 77.

(100) A vaccine against SARS-CoV-2 infection comprising the polypeptide antigen of embodiment 77.

The above written description of the invention provides a manner and process of making and using it such that any person skilled in this art is enabled to make and use the it same, this enablement being provided in particular for the subject matter of the appended claims, which make up a part of the original description,

The above description is presented to enable a person skilled in the art to make and use the invention, and is provided in the context of a particular application and its requirements, Various modifications to the preferred embodiments will be readily apparent to those skilled in the art, and the generic principles defined herein may be applied to other embodiments and applications without departing from the spirit and scope of the invention, Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLE 1

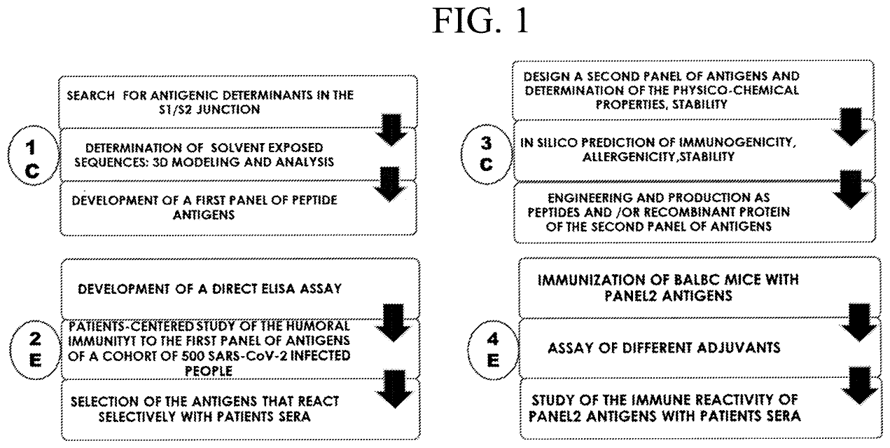

Materials and Methods. A four-phase strategy that combined empirical, computational and experimental approaches was developed. FIG. 1 describes the workflow followed to implement this strategy.

1. Cohort collection & Sera Preparation: A cohort of 492 volunteers who were COVID19 affected persons and who tested positive for SARS-CoV-2 using specific PCR assay was recruited. All the volunteers involved in this study were workers of south Asian origin. Comprehensive data (biometric, clinical and some genetic data) of each participant was collected. 81 healthy persons with no suspected COVID19 symptoms or SARS-CoV-2 PCR confirmed infection were recruited as controls. The members of this control group were collected in the same geographic area (Bahrain) at the peak of the pandemic. The sera were prepared and conserved at −20° C. 100 pre-pandemic sera of healthy individuals (Historical sera, collected well before the emergence of SARS-CoV-2 epidemics) were also retrieved. Thirty-seven WHO international reference sera, Anti-SARSCoV-2 Verification Panel for Serology Assays NIBSC code: 20/B770 (14 negative and 23 positive sera) were also acquired from the NIBSC The National institute for Biological Standards and Control, UK). The SARS-CoV-2 PCR test data corresponding to all these reference sera was not available. The data are compiled in Table 1.

TABLE 1
SARS-CoV-2 INFECTED AND HEALTHY DONORS:
BAHRAIN COHORT CHARACTERISTICS

| | | | INFECTED/ |
|---|---|---|---|
| | | | COVID-19 |
| | | HEALTHY | PATIENTS |
| AGE (years) | | | |
| Average | | 40.7 | 43.0 |
| Range | | 21-66 | 21-68 |
| GENDER | | | |
| Male | | 69 | 478 |
| Female | | 12 | 14 |
| ETHNICITY | | | |
| Caucasian | | 50 | 0 |
| Asian | | 31 | 492 |
| PCR POSITIVITY | | NA | 492 |
| Range of sampling time from | | NA | 2.5 to 55 weeks |
| date of positive PCR testing | | | |
| code | DISEASE STATUS | | |
| 0 | Asymptomatic | NA | 310 |
| 1 | Mild | NA | 122 |
| 2 | Moderate | NA | 23 |
| 3 | Severe/Critical | NA | 34 |

| | |
|---|---|
| SAMPLE COLLECTION PERIOD | January to June 2020 |

2. Analysis of SARS-CoV-2 S protein S1/S2 junction sequence Pr solvent exposure: The BepiPred-2.0: Sequential B-Cell Epitope Predictor server (hypertext transfer protoeol://www.cbs.dtudk/services/BepiPredlcite.pirp) was used to study the secondary, structure, determine residue exposure and identify B-cell epitopes on the SARS-CoV-2 S is spike protein S1/S2 junction sequence (residues positions 588-764). BepiPred-2.0 is based on a random forest algorithm trained on epitopes annotated from antibody-antigen protein structures (Jespersen el al. 2017). The PDB structure's of the complex formed between the Covid-19 Spike protein Receptor Binding Domain (RBD) and the human ACE2 receptor (PDB ID 6LZG), and the Spike Protein complex (PDB ID 6VXX) were downloaded from the RCSB database (hypertext transfer protocol secure://www.resb.org). The structure was cleaned of water and heteroatoms and the complex split it into separate PDB files using Pymol software (Schrodinger) for sequence visualization ant annotation of solvent-exposed areas of the protein target region.

3. Prediction of immunogenic peptides: For the prediction of potential candidate for B-cell response, the Spike junction sequence (residues positions 588-764) containing the proteolysis site and the sequence (residues positions 778-809) containing the TMPRSS2 proteolysis site were submitted to Prosper to select the immunogenic determinants. Peptides of inter st including P3, P3L, P4/TMP and SJ/FT have been selected or engineered taking into account these proteolysis sites. Prosper is an integrated feature-based server for in silico identification of protease substrates and their cleavage sites for twenty-four different proteases (hypertext transfer protocol://lightning.med.monash.edu.au/PROSPER/). The sequence was also run on the improved Proteasome Cleavage Prediction Server 2 (hypertext transfer protocol://imed.med.ucm.es/Tools/pcps/). This server determines the cleavage sites within a protein generated by the constitutive proteasome or the immunoproteasome using different Ngram models (Garcia-Boronat, et al. 2008: Song el al., 2012). 4. 3D Modeling of immunogenic peptides: For structure ab initio modelng, i-Tasser (hypertext transfer protocol secure://ibanglab,dcmb.med,umich,edu) was employed. Modeling alproack I-TASSER (Iterative Threading Assembly Refinement) is a hierarchical approach to protein structure prediction and structure-based function annotation, It first identifies structural templates from the PDB by multiple threading approach LOMETS, with full-length atomic models constructed. by iterative template-based fragment assembly simulations. Function insights of the target are then derived by, re-threading the 3D models through protein function database

5. Prediction of peptide binding to AMC class I and Class II alleles: The NetMHCpan 2.3 Server (hypertext transfer protocol://www.cbs.dtu.dk/servicestiNetMHCpan-2.3/) was used to determine binding of antieenic: peptides to MHC class I. molecules of known sequence using artificial neural networks (ANNs). The method is trained on a combination of more than 180,000 quantitative binding data and MS derived MHC eluted ligands. Binding affinity is predicted based on hunting data covering 172 MHC molecules from human (HLA-A, B, C, E), mouse (H-2), cattle (BoLA), primates (Patr, Martin, Gogo) and swine (SEA), in addition, the server Propred1 (hypertext transfer protocol://www.imtech.res.in/raghava/propred1/) was used to analyze peptides binding to MHC class-I alleles. The method is a .matrix-based method that allows the prediction of MTC binding sites in an antigenic sequence for 47 MHC class-I NetNHCII 2.3 server (hypertext transfer protocol://www.cbs.dtu.dk/services/NetMECII) was used to predict the binding of peptides to MHC class-II alleles HLA-DR, HLA-DQ, HLA-DP. For all predictions, the S protein S1/S2/S′2 junction (residue 588-764) and (residues 778-809) sequences were analyzed, the peptides showing binding affinity for the different MHC molecules were selected.

6. Peptides sequences and syntheis: Peptides lengths varied from 9 to 64 amino acid residues with three peptides: P2, P3 and P21 displaying respectively 2 [P2] and 3 repetitions [P-3, P-21] of the core antigenic sequence 16 and 10 AA respectively, Peptides were synthetized chemically by GeneCust (Geneeust, Boynes, France) using solid-phase organic synthesis. Peptides were synthesized at a 5 mg scale at 85% minimum purity. Peptides purity to and integrity were checked using Mass-Spectrometry analysis and High-Performance Liquid Chromatography (HPLC). Peptides were produced and lyophilized until further use.

7. Indirect ELISA assay developmeni: 96-well enzyme-linked immunosorbent assay (ELISA) plates were coated (Nunc MaxiSorp, Thermo Fisher Scientific) overnight with 200 per well of streptavidin in PBS buffer (see subpart (a) below). Then the coated plate was washed and blocked With PBS containing 5% non-fat milk powder at room temperature for 1 hours. After blocking, the plates were washed three times with 200 μl Wash Buffer. The plates were then dried on absorbent paper and proceeded immediately with peptide coating (see subpart (b) below). All serum samples were tested at a dilation of L100 in dilution buffer with incubation at. room temperature liar 1-hour. After 3 times of extensive washing with PBS containing 0.05% Tween 20, 100 UL of HRP-conjugated goat anti-human IgG (1:10000, abeam) was added and incubated for 1 hour at room temperature. The ELISA plates were then washed 3 times with PBS containing 0.05% Tweet 20. Subsequently, 100 μL, of HRP substrate (TMB, Sigma-Aldrich) was added into each well. After 20 minutes of incubation the reaction was stopped by adding 100 μL of 2N H₂SO₄ solution and analyzed on a FLUOstar Omega (BMG LABTECH) absorbance microplate reader at 450 nm wavelength (see subpart (c) below). A sample was defined as IgG positive when the OD value was 3 standard. deviations (SD) above the mean of the negative controls (n=100).

The use of peptides in solid-phase immunoassays requires an efficient method for immobilization of the peptides on the solid phase, i.e. a method which does not depend on the amino acid sequence of the peptide being tested. For this purpose, bionnylation of multiple synthetic peptides, and snbsequent attachment of the peptides to a plastic surface coated with streptavidin is used. Subparts (a)-(c) describe the employed method:

a. Streptavidin Plate-Coaling
Reagents:
Streptavidin (5 mg, SA101, Merck)
Tween 20 (P2287-500 ML, Sigma-Aldrich)
Sodium chloride (S3014-1 KG, Sigma-Aldrich)
Nonfat skimmed milk “Regilait” powder
Sodium phosphate monobasic (S0751. Sigma-Aldrich)
Sodium phosphate dibasic (S0876, Sigma-Aldrich)
Milli-Q grade Water

Buffers
Phosphate, 0.01 M, pH 7.2

TABLE 2
Required components

| Component | Amount | Concentration |
|---|---|---|
| Na₂HPO₄ (mw: 141.96 g/mol) | 9.45 g | 0.0666M |
| NaH₂PO₄ (mw: 119.98 g/mol) | 4 g | 0.0334M |

a) Prepare 800 mL of distilled water in a suitable container.
b) Add 9.45 g of Na₂HPO₄ to the solution.
c) Add 4 g of Na₂HPO₄ to the solution.
d) Adjust solution to final desired pH using HCl or NaOH
e) Add distilled water until volume is 1 L
Wash Si.dkr: 0.01M phosphate. 0.15 M sodium chloride, pH 7.2, 0.05% Tween 20
Blocking Buffer: Wash Buffer with 5% skimmed milk (w/v)

Methods

Streptavidin Coating (4steps)
a) A solution of streptavidin in phosphate buffer 0.01M, pH 7.2 was prepared at the desired concentration (200 ng of streptavidin by well).
b) 100 μl of streptavidin solution was incubated into the wells at 4° C. overnight.
c) The wells were washed three times with 200 μl Wash Buffer and blocked with 100 μl Blocking Buffer for 1 hr at room temperature (RT).
d) The wells were subsequently washed three times with 200 μl Wash Buffer. Plates are then dried on Absorbent paper and used immediately for peptide coating.

b. Peptide coating for SARS COPT-2 ELLS:4 Optimization Reagents:
Biotinylated synthetic peptides (5 mg, parity >75%)
DMSO (dimethyl sulfoxide) (41639, Sigma-Aldrich)
Tween 20 (P2287, Sigma-Aldrich)
Sodium phosphate monobasic (S075 Simla-Aldrich)
Sodium phosphate dibasic (S0876, Sigma-Aldrich)
Sodium chloride tS3014, Sigma-Aldrich)
Nonfat skimmed milk “Regilait” powder
Milli-Q grade Water

Buffers
Phosphate, 0.01M, pH 7.2 (see 7.a, above)
Wash Buffer; 0.01M phosphate. 0.15 M sodium chloride, pH 7.2, 005% Tween 20
Blocking Buffer: Wash Buffer with 5% skimmed milk (w/v)
Diluent Buffer: Wash Buffer with 5% skimmed milk (w/v)

Method

Peptide resuspension and coating (4 steps)
a) The biotinylated peptides were supplied as a dry powder (5 mg/tube). The identity of the peptide in each tube was given in the information supplied with the peptides. The peptides were reconstituted in 500 ul (mother solution 10 mg/ml) of DMSO (dimethyl sulfoxide)
b) Before coating, the peptide was diluted to a final concentration of 2 μg/ml and with Diluent Buffer (2 μl from peptide mother solution in 10 ml diluent buffer).
c) To react the streptavidin coated, milk-blocked plate prepared as described in 6a. above with the biotinylated peptides, 100 μl of the diluted peptide solutions were transferred into the corresponding wells, the plate was placed on an orbital shaker at 100 rpm and the reaction allowed to proceed for 1 hr at mom temperature.
d) The wells were washed three times with 200 μl Wash Buffer. After the washings, the. est of washing buffer was removed from the wells by vigorously “slapping” the plates, wells down, on a bench top covered with an absorbent material (paper towels).

c. Indirect SARS COV-2 IgG Peptide Elisa Protocol Reagents:
Tween 20 (P2287, Sigma-Aldrich)
Sodium phosphate monobasic (S0751, Sigma-Aldrich)
Sodium phosphate dibasic (S0876, Sigma-Aldrich)
Sodium chloride (S3014, Sigma-Aldrich)
nonfat skimmed milk “Regilait” powder
Goat Anti-Human IgG Fc (HRP) (ab97225, abeam)
3,3′,5,5′-tetramethylbenzidine (IMB) (CL07, Sigma-Aldrich)
Sulfuric acid (2N) (1603131000, SIGMA)
Milli-Q grade Water

Buffers
Phosphate, 0.01M, pH 7.2 (see 7.a. above)
Wash Buffer; 0.01M phosphate, 0.15 M sodium chloride, pH 7.2, 0.05% Tween 20
Diluent Buffer: Wash Buffer with 5% skimmed milk (w/v)
Stop solution: H₂SO₄ (2N)

Methods

Indirect ELISA Protocol (9 steps)
a) The serum to be tested according to the plate plan using Diluent Buffer (Use serum dilution 1/100) was diluted. The optimum dilution of the serum depends to some extent on the source and the level of antibodies present in the sample.
b) 100 μl of the diluted serum was added to each of the wells of the plate containing captured peptides prepared as described in 7.b. above The plate was placed on a shaker at .100 rpm and incubated for thr at Room Temperature.
c) The wells were washed three times with 200 μl Wash Buffer. After washing, the rest of wash buffer was removed from the wells by vigorously “slapping” the plates, wells down, on a bench top covered with an absorbent material (paper towels).
d) 100 μl of horseradish peroxidase labeled anti-IgG (1/1000 of 1 mg/ml solution) was added to each well and the plate was incubated for 1 hr at Room Temperature.
e) The wells were washed three times with 200 μl Wash Buffer (as in step c)
f) 100 μl of TMB substrate was added to each
g) The plate was then incubated. for 20 mins at room temperature, protected from light.
h) The reaction was stopped with 100 μl of 2 N H₂SO₄
i) The plate was then read at 450 mu using an ELISA plate-reader.

8. Construction of the multi-epitope subunits: (FIG. 8A-8N4) in addition to the P3 polypeptide, the P3-AD, the P3-L, and the P3-LSAD subunits were designed for immunization by connecting three copies of the antigenic peptide encompassing the B cell epitopes, core motif 16 amino acid for P3-AD and 32 residues for P3-1. and P3-L/Ad using the short linker GGGGS. The ILR agonist β delensin 1 was also added at the COOH terminal to serve as an adjuvant for enhancing the immunogenicity (Yang et at., 1999). Six additional subunits (SJ 80, SJ100, SJ120, SS-FT, SJ-S-2/FP and SJ-S′2/FP1-2) were also designed, as well as their β defensin 1-adjuvated (AD) forms. The cDNAs nucleotide sequence encoding for these multi-epitope subunits were synthetized chemically by GeneCust (Genecust, Boynes, France),

9. Prediction of the vaccine subunits antigenicity and allergenicity: The Vaxiien server (hypertext transfer protocol://wwl,v,d4g-phannfachetivaxljeniVaxiJen/VaxiJen.html 1.s. was used to evaluate the ithmunogenicity of. he multi-epitope subunits that were designed.

To check for potential allergenicity of these subunits, the AllerTOP v2.0 (hypertext transfer protocol//www.ddg-pharmfac.oet/AllerTOP/and AlgPred Server (hypertext transfer protocol://crdd.osdd.net/raghava/algpred/) (Saha & Raghava, 2006) were used.

10. Prediction of the vaccine subunits physiological characteristics: The ProtParam tool of the ExPASy database server (hypertext transfer protocol://web.expasy.org/protparam/ was used to determine the physicochemical parameters of the vaccine protein subunits.

11. Production of Polypeptide Antigens PL3 and SJ-FT recombinant protein 1-Protein Name: P L 3

Protein Sequence:

(SEQ ID NO: 170)
MASYQTQTNS PRRARSVASQ SIIAYTMSLG AENGGGGSAS
YQTQTNSPRR ARSVASQSII AYTMSLGAEN GGGGSASYQT
QTNSPRRARS VASQSIIAYT MSLGAENLEHHHHHH
(same as SEQ ID NO: 43 + His tag)

Poly H tag
Molecutar weight 12.2 KDa
PI: 10.25
Molar Extinction coefficient:
Extinction coefficients are in units of M-1 cm-1, at 280 nm measured in water.
Ext. coefficient 8940
Abs 0.1% (=1 g/l) 0.735
Expression Vector's name: pET-22b (+)
Resistance: Ampicillin
Gene insertion site: NdeI-XhoI
Expression Host: E. coli BL21 (DE3)
Purity: 90%
Quantity: 4 mg
Storage Buffer: 50 mM Hepes, 300 mM NaCl, 0.1% SKL, pH8.0
Tag removal: no
Endotoxin: <1EU/ug
Experimental materials
LB, liquid medium, Ampicillin
IPTG
Protein purification reagent
1) Lysis Buffer (Hepes+Urea
2) Washing Buffer (Hepes+Urea+imidazole)
3) Elution Buffer (Hepes+Urea+imidazole)
4) SDS-PAGE related reagents (Sin harm)
5) Protein concentration quantitation kit (Nanodrop Method, Thermo)
6) Storage Buffer (50 mM Hepes, 300 mM NaCl 0.1%, SKL, pH8.0)
Main experimental equipment

Electrophoresis System (Talton)

Gel Documentation and Analysis System (Tanon)

A half-dry rotary (VE386)

spectrophotometer (Shanghai ShunYuHengPing)
Experimental methods

(A) Transformation

1) The recombinant expression Vector was transformed into competent cell.
BL21 (DE3). Add 1 μL plasmid into 100 ul competent cell (ice bath 20 min);

2) Heat shock 90s at 42° C., put in to ice bath 5 m in quickly, add 6001 μL LB medium;

3) Shook 1h with 220 rpm at 37°C., dip-coating on 50 μg/mL, A+LB plate after centrifuged, the inversion cultures at 37° C. overnight,

(B) The identification of target protein

1) Pick up the single clone, inoculated into the test tube of 4 mL LB medium (50 μg/mL A+), 220 rpm shook over night at 37° C.;

2) Next day, inoculated into 30 mL LB (50 μg/mL A+) with 1: 100, 37° C. 220 rpm shook. until the OD 600 of bacteria is 0.6-0.8 (4 h)

3) Draw out 1 mL culture, centrifuge 2 min with 10000 g, (room temperature), prepared the Un-induced Sample;

4) Inoculated several single colonies into 4 mL LB medium (50 μg/mL A+), Add IPTG into these tithes, the final concentration is 0.5 mM, shook 4 h (37° C. 220 rpm), induce the expression of target protein. Prepared the induced sample and analysis by.SDS-PAGE. FIG. 10A and 10B respectively show the stained SDS-PAGE gel and Western Blot, M: marker proteins, Lane 1: Uninduced sample, Lands 2-6 induced samples,

(C) The analysis og sohthle target protein. Take out 2 mL culture, centrifuge 2 min with 10000 g (room, temperature), throw away supernatant, resuspended the precipitate with 1000 μL Storage Buffer, and sonicate (Φ3, 15%, 2s on/8s off, 5 min). Analysis supernatant and precipitate by SDS-PAGE. FIG. 11, Results of Optimization and solubility analysis.

M: Protein Marker

| | |
|---|---|
| 1: | Induced with 0.2 mM IPTG at 15° C. |
| 2: | Induced with 1.0 mM IPTG at 15° C. |
| 3: | Induced with 0.2 mM IPTG at 37° C. |
| 4: | Induced with 1.0 mM IPTG at 37° C. |
| 5: | Un-induced sample |
| 6: | Precipitate induced with 1.0 mM IPTG at 37° C. |
| 7: | Supernatant induced with 1.0 mM IPTG at 37° C. |
| 8: | Precipitate induced with 0.2 mM IPTG at 37° C. |
| 9: | Supernatant induced with 0.2 mM IPTG at 37° C. |
| 10: | Precipitate induced with 1.0 mM IPTG at 15° C. |
| 11: | Supernatant induced with 1.0 mM IPTG at 15° C. |
| 12: | Precipitate induced with 0.2 mM IPTG at 15° C. |
| 13: | Supernatant induced with 0.2 mM IPTG at 15° C. |

(D) Affinity purification 600 of bacteria is 0.6-0.8. Add IPTG (the final concentration is 1.0 mM) into culture, shook 4 h with 220 rpm at 37° C., then collect bacteria with 8000 rpm at room temperature.

2) Throw away medium, resuspended the cells with 100 ml Lysis Buffer, sonicate (Φ15, 50%, 3s on 6s off,6 min). Then centrifuge 30 min with 16000 rpm at 4° C.,

3) Add 2 mL Ni-NTA into supernatant, incubate at 4° C. for 1h.

4) Take out all of the reaction product into column, collect flow through;

5) Wash the Ni-NTA with 10 CV Washing Buffer.

6) Elute the Ni-NTA with Elution Buffer. FIG. 12 shows results of SDS-PAGE of affinity purified materials.

(E) Dialysis: Dialyze target protein into Storage Buffer (50 mM Hepes, 300 mM NaCl, 0.1% SKL, pH8.0) at 4° C.

(F) The Endotoxin removal and Endotoxin Level test

1) Wash the Endotoxin Removal Beads with Regeneration Buffer in 2 CV, and then equilibrate the Beads with the Storage Buffer in SCV;

2) Load the target protein onto the equilibrated Beads, collect the flow tough;

3) Pool the fractions after SDS-PAGE, Endotoxin Level was less than 1 EU/μg as determined by TAL test.

4) Split the protein and then perform freezing.

(G) SDS-PAGE analysis. FIG. 13 shows an SDS-PAGE of the purified protein. M corresponds to the Protein Marker, S corresponds to the target protein, the lower right arrow indicates the position of the protein.

(H) Concentration determination. The protein concentration was determined by the Bradford method. FIG. 14 depicts a Bradford Standard Curve.

1-Standard curve, see FIG. 14.

2-Protein concentration calculation. Repeat three tests, take the average value of OD595, substitute into the equation, get the concentration value: 1 mg/mL.

3-Storage Store at −80° C.

2-Pmtein Name:-S1-FT

Molecular weight: 26 KDa

PI: 5.63

Extinction. coefficient 17920

Abs 0.1% (=1)g/l 0.688, assuming all pairs of Cys residues form cystines

Ext. coefficient 17420

Abs 0.1% (=1g/l) 0.669, assuming all Cys residues are reduced

Protein sequence:

(SEQ ID NO: 171
MECDIPIGAG ICASYQTQTN SPRRARSVAS QSIIAYTMSL
GAENSVAYSN NSIAIPTNFT
ISVTTEILPVSMTKTSVDCTMYICGDSTECSNLLLQYGSFCTQLNRALT
GIAVEQDKNTQ EVFAQVKQIYKTPPIKDFGG FNFSQILPDP
SKPSKRSFIE DLLFNKVTLA DAGFIKQYGD
CLGDIAARDLICAQKFNGLTVLPPLLTDEMIAQYTSALLA
GTITSGWTFG ALEHHHHHH;

similar to SEQ ID NO: 1.44 but has N terminal M and C terminal LEHHHHHHH).

Expression vector information. Vector's name: pET-22b (+) Resistance: Ampicillin Gene insertion site: NdeI-Xhol

Experimmtal methods

(A) Transformation

1) The recombinant expression vector was transformed into E. coli competent cell BL21 (DE3). Add 1 μL plasmid into 100 μl competent cell (ice bath 20 min),

2) Heat shock 90 s at 42° C., put in to ice bath 5 min quickly, add 600 μl LB medium;

3) Shook 1 h with 220 rpm at 37° C. dip-coating on 50 μg/mL A LB plate after centrifuged, the inversion cultures at 37° C. overnight.

(B)The expression identification of target protein

1) Pick up the single clone, inoculated into the test tube of 4 mL LB medium (50 μg/mL A), 220 rpm shook over night at 37° C.;

2) Next day, inoculated into 30 mL: LB (50 μg/mL A) with 1: 100 37° C. 200 rpm shook until the OD₆₀₀ of bacteria is 06-0.8 (4 h);

3) Draw out 1 mL culture,centrifuge with 10000 g (root temperature), prepared the Un-induced Sample;

4) Inoculated several single colony into LB medium (50 μg/mL A), Add IPTG into the these tubes, the final concentration is 0.5 mM, shook 4 h (37° C. 220 rpm), induce the expression of target protein, Prepared the induced Sample and analysis by SDS-PAGE, FIG. 15A and 15B show the expression evaluation of SDS -PAGE and Western Blotting, M: Protein Marker; Lane 1: tin-inducing sample; Lanes 2-6: Inducing sample

(C) The soluble analysis of target protein. Take out 2 mL culture, centrifuge 2 min with 10000 g (room temperature), throw away supernatant, resuspended the precipitate with 1000 μL Storage Buffer, and sonicate Φ3, 15%, 2 s of/8 s off, 5 min), Analysis supernatant and precipitate by SDS-PAGE, FIG. 16 shows SOS-PAGE results of optimization and solubility analysis.

M: Protein Marker

| | |
|---|---|
| 1: | Induced with 1.0 mM IPTG at 15° C. |
| 2: | Induced with 0.2 mM IPTG at 15° C. |
| 3: | Induced with 1.0 mM IPTG at 37° C. |
| 4: | Induced with 0.2 mM IPTG at 37° C. |
| 5: | Un-inducing sample |
| 6: | Precipitate induced with 0.2 mM IPTG at 37° C. |
| 7: | Supernatant induced with 0.2 mM IPTG at 37° C. |
| 8: | Precipitate induced with 1.0 mM IPTG at 37° C. |
| 9: | Supernatant induced with 1.0 mM IPTG at 37° C. |
| 10: | Precipitate induced with 0.2 mM IPTG at 15° C. |
| 11: | Supernatant induced with 0.2 mM IPIG at 15° C. |
| 12: | Precipitate induced with 1.0 mM IPTG at 15° C. |
| 13: | Supernatant induced with 1.0 mM IPTG at 15° C. |

(D) Affinity Purification

1) Inoculated into 3L LB, 37° C. 220 rpm shook until OD₆₀₀ of bacteria is 0.6-0.8 Add IPTG (the final concentration is 0.2 mM) into culture, shook 16 h with 220 rpm at 15° C., then collect bacteria with 5000 rpm at room temperature.

2) Throw away medium, resuspended the cells with 150 ml Lysis Buffer, sonicate (Φ10, 25%, 2s on 8s off 10 min). Then centrifuge 10 min with 16000 rpm at VC,

3) Add 2 mL Ni-NTA into supernatant, incubate at 4° C. for 1 h.

4) Take out all of the reaction prduct into column, collect flow through;

5) Wash the Ni-NTA with 5CV Washing Buffer.

6) Elute the Ni-NTA with Elution Buffer. See FIG. 17,

(E) Dialysis

1) Dialyze target protein into Storage Buffer (20 mM Tris, 4 M Urea, pH8.0) at 4° C.

2) Split the protein.

(F) Endotoxin Removal

1) Equilibrium 5 mL Endotoxin Removal Beads.

2) Take out all of protein sample into column, collect flow through:,

3) Split the protein and perform the Endotoxin test.

SDS-PAGE, Take the protein sample, run SDS PAGE, as shown in the figure below. FIG. 18 shows the. SDS-PAGE result of the purified protein, M. corresponds to the Protein Marker, S corresponds the target protein, the red arrow indicates the position of the protein. Concentration determination. The protein concentration was determined by the Nanodrop Method: (A) Protein concentration calculation was 0.6 mg/mL, (B) Storage: Stored at −80°C.

### EXAMPLE 2

### Immunization of Mice

Mice immunization: For this study: Female Balb/c. mice at 6-8 weeks of age were used. The experiments were conducted at the animal facility of AGU; animal experimentation was conducted according to national and international guidelines such as those issued by OLAW (Office of Laboratory Animal Welfare). The Ethics Committee of the Arabian Gulf University approved all protocols. Each group of mice was composed of 5 animals. The animals were immunized subcutaneously. Two protocols were evaluated using respectively, 200 μgr of peptide such as P3 (SEQ ID 43) or P4/TMP-Pol3 (SEQ ID 116) emulsified in complete Freund's adjuvant and with 30 μgr of CpG (ODN 2395 VaceiGrade, InvivoGen, USA). PBS was used for the immunization of the control group and administered a boost shot with 200 μgr of peptide emulsified in incomplete Freund's adjuvant on days 14. Blood samples were collected before immunization, and at 14, 28- and 42-days post-injection. The sera samples were tested for specific IgG against P3 or P4/TMP-Pol3 using the indirect ELISA test described in section 7 above (see specifically 7.a.-7.c.), Briefly, mice sera at appropriate dilutions were added to streptavidin plates coated with the peptide P3. After incubation under adequate conditions, horseradish peroxidase (HRP) conjugated goat anti-mouse IgG was used as secondary antibodies.

Data analysis: The data was analyzed using the R (v 4.0.5) environment and the readxl (v1.3.1), dpiyr (v 1.0.6) and stringi (v 1.5.3) libraries software packages for data handling, The “Disease Status” was considered variable as a factor type (categorical variable). We transformed the optical density values [OD] obtained from the MASA experiments from numeric (float) type to factor type (categorical variable) using the following rule (The 0D value threshold); OD<0.45 immune (−) and OD>=0.45=Immune (+), for the epitopes displayed by the antigen. To measure the eventual association between the two variables, a Chi-square test (chisq.test function from the Stats Package) was performed. Because some oaf the expected values were below 5 when considering the variables as independent, the Fisher's exact test (fisher test function from the Stats package) was used to compute the p value.

### Results

I-Analysis of the SARS-CoV-2 S protein S1/S2 Subunit Junction for Solvent-Exposed Region:

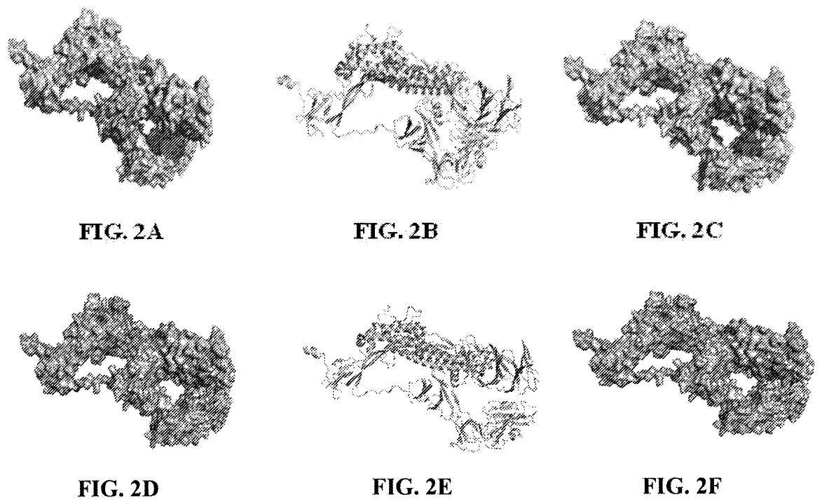

To identify the solvent accessible regions in the SARSCoV-2 S protein sequence, the two available SARS-CoV-2 spike glycoprotein Cryo-EM structures (PDB; 6VSB and 6VXX) were used. However, the two structures are incomplete, display several gaps, and are missing the structure of the furin cleavage sites [FCS] (Walls, et at. 2020; Wrapp et. al. 2020). Therefore, we considered the regions surrounding the Furin and the TMPRSS2 cut sites to be accessible and thus the site should be solvent exposed. Indeed, the cleavage of the S protein into S1 and S2 moiety by the cellular fruit and the fusion. with the virus host-cell receptor would otherwise not happen. As shown in FIG. 2, the prediction confirmed that the S1/S2 Junction region is surface exposed and contains several potential epitopes as suggested by the estimated epitopes probabilities (Table 3).

TABLE 3
Prediction of the solvent accessibility by BepiPred-
2.0 (Sequential B-Cell Epitope Predictor) of the SARS-
CoV-2 S protein S1/S2/S′2 subunits junction.

A- The region surrounding the furin cut motif contain 3 solvent exposed
sequences predicted to contain B-Cell linear epitopes, shown in purple
(E648-G654), in blue (Q662-S676) and in yellow (T683-N696). The furin
cut site motif, PRRAR sequence (SEQ ID NO: 107) is displayed in red.
B- The region surrounding the TMPRSS2 cut motif contain exposed
residues and antigenic epitopes sequences, shown in yellow
(P794-R802), in blue (I805-V813) and in purple (R834-L845).

The probabilities of the corresponding sequences to contain antigenic epitopes are highlighted in light Green, AA=amino acid, E=exposed, B=Burried, RSA=Relative Surface Accessibility, Prob.=Probability. The sequence or the S1/S2 subunits junction read down the column labeled AA corresponds to residues 1-59 of SEQ ID NO: 63.

TABLE 3A

| Position | AA | E/B | RSA | Helix Prob. | Sheet Prob. | Coil Pro | Epitope Prob. |
|---|---|---|---|---|---|---|---|
| 648 | E | E | 0.55 | 0.022 | 0.552 | 0.426 | 0.56256 |
| 649 | C | B | 0.072 | 0.022 | 0.359 | 0.619 | 0.55878 |
| 650 | D | E | 0.608 | 0.021 | 0.279 | 0.699 | 0.53844 |
| 651 | I | E | 0.34 | 0.056 | 0.142 | 0.802 | 0.516 |
| 652 | P | E | 0.304 | 0.052 | 0.084 | 0.864 | 0.49978 |
| 653 | I | B | 0.226 | 0.056 | 0.142 | 0.802 | 0.47911 |
| 654 | G | E | 0.385 | 0.056 | 0.142 | 0.802 | 0.474 |
| 655 | A | B | 0.21 | 0.056 | 0.142 | 0.802 | 0.46133 |
| 656 | G | B | 0.085 | 0.069 | 0.386 | 0.545 | 0.44933 |
| 657 | I | B | 0.081 | 0.079 | 0.592 | 0.329 | 0.45411 |
| 658 | C | B | 0.028 | 0.074 | 0.484 | 0.442 | 0.46522 |
| 659 | A | B | 0.28 | 0.064 | 0.216 | 0.721 | 0.48833 |
| 660 | S | E | 0.406 | 0.052 | 0.084 | 0.864 | 0.52478 |
| 661 | Y | B | 0.311 | 0.053 | 0.043 | 0.903 | 0.55044 |
| 662 | Q | E | 0.365 | 0.018 | 0.088 | 0.893 | 0.57533 |
| 663 | T | E | 0.361 | 0.02 | 0.205 | 0.775 | 0.604 |
| 664 | Q | E | 0.449 | 0.022 | 0.359 | 0.619 | 0.61522 |
| 665 | T | E | 0.409 | 0.021 | 0.451 | 0.528 | 0.63322 |
| 666 | N | B | 0.266 | 0.022 | 0.552 | 0.426 | 0.634 |
| 667 | S | E | 0.372 | 0.022 | 0.552 | 0.426 | 0.62044 |
| 668 | P | B | 0.265 | 0.022 | 0.552 | 0.426 | 0.63311 |
| 669 | R | E | 0.345 | 0.021 | 0.451 | 0.528 | 0.62922 |
| 670 | R | E | 0.389 | 0.021 | 0.451 | 0.528 | 0.63478 |
| 671 | A | B | 0.235 | 0.022 | 0.359 | 0.619 | 0.63556 |
| 672 | R | E | 0.416 | 0.022 | 0.359 | 0.619 | 0.637 |
| 673 | S | E | 0.559 | 0.02 | 0.205 | 0.775 | 0.62756 |
| 674 | V | E | 0.537 | 0.019 | 0.141 | 0.84 | 0.60311 |
| 675 | A | E | 0.364 | 0.02 | 0.205 | 0.775 | 0.57 |
| 676 | S | E | 0.411 | 0.021 | 0.279 | 0.699 | 0.55611 |
| 677 | Q | B | 0.199 | 0.004 | 0.514 | 0.481 | 0.52 |
| 678 | S | B | 0.14 | 0.021 | 0.756 | 0.223 | 0.49722 |
| 679 | I | B | 0.118 | 0.018 | 0.846 | 0.136 | 0.48333 |
| 680 | I | B | 0.142 | 0.018 | 0.848 | 0.136 | 0.49383 |
| 681 | A | B | 0.113 | 0.021 | 0.756 | 0.223 | 0.49744 |
| 682 | Y | B | 0.167 | 0.021 | 0.451 | 0.528 | 0.52367 |
| 683 | T | E | 0.308 | 0.021 | 0.279 | 0.699 | 0.55233 |
| 684 | M | E | 0.318 | 0.019 | 0.141 | 0.84 | 0.58244 |
| 685 | S | E | 0.412 | 0.018 | 0.088 | 0.893 | 0.61144 |
| 686 | L | E | 0.386 | 0.052 | 0.084 | 0.864 | 0.64167 |
| 687 | G | E | 0.407 | 0.056 | 0.142 | 0.802 | 0.65711 |
| 688 | A | E | 0.398 | 0.056 | 0.142 | 0.802 | 0.67133 |
| 689 | E | E | 0.332 | 0.064 | 0.216 | 0.721 | 0.66878 |
| 690 | N | E | 0.378 | 0.021 | 0.279 | 0.699 | 0.676 |
| 691 | S | E | 0.334 | 0.021 | 0.279 | 0.699 | 0.66289 |
| 692 | V | B | 0.238 | 0.022 | 0.359 | 0.619 | 0.64889 |
| 693 | A | E | 0.395 | 0.021 | 0.279 | 0.699 | 0.63422 |
| 694 | Y | E | 0.329 | 0.019 | 0.141 | 0.84 | 0.61322 |
| 695 | S | E | 0.382 | 0.02 | 0.205 | 0.775 | 0.55722 |
| 696 | N | E | 0.352 | 0.005 | 0.336 | 0.66 | 0.53678 |
| 697 | N | B | 0.195 | 0.004 | 0.514 | 0.481 | 0.48589 |
| 698 | S | B | 0.241 | 0.002 | 0.816 | 0.182 | 0.45978 |
| 699 | I | B | 0.044 | 0.001 | 0.9 | 0.099 | 0.44489 |
| 700 | A | B | 0.233 | 0.001 | 0.9 | 0.099 | 0.43922 |
| 701 | I | B | 0.078 | 0.023 | 0.655 | 0.322 | 0.43211 |
| 702 | P | B | 0.109 | 0.005 | 0.336 | 0.66 | 0.43644 |
| 703 | T | B | 0.283 | 0.019 | 0.141 | 0.84 | 0.43644 |
| 704 | N | E | 0.459 | 0.019 | 0.141 | 0.84 | 0.46111 |
| 705 | F | B | 0.149 | 0.022 | 0.359 | 0.619 | 0.46067 |
| 706 | T | E | 0.33 | 0.021 | 0.756 | 0.223 | 6.48211 |

TABLE 3B

| Position | AA | E/B | RSA | Helix Prob. | Sheet Prob. | Coil Pro | Epitope Prob. |
|---|---|---|---|---|---|---|---|
| 778 | T | B | 0.161 | 0.056 | 0.142 | 0.802 | 0.58544 |
| 779 | P | E | 0.375 | 0.019 | 0.141 | 0.84 | 0.58478 |
| 780 | P | E | 0.436 | 0.02 | 0.205 | 0.775 | 0.58744 |
| 781 | I | B | 0.208 | 0.021 | 0.279 | 0.699 | 0.60178 |
| 782 | K | E | 0.459 | 0.02 | 0.205 | 0.775 | 0.59756 |
| 783 | O | E | 0.417 | 0.019 | 0.141 | 0.84 | 0.60378 |
| 784 | F | B | 0.21 | 0.018 | 0.088 | 0.893 | 0.60444 |
| 785 | G | E | 0.441 | 0.018 | 0.047 | 0.935 | 0.58767 |
| 786 | G | E | 0.456 | 0.018 | 0.088 | 0.893 | 0.58433 |
| 787 | F | B | 0.156 | 0.021 | 0.279 | 0.699 | 0.57089 |
| 788 | N | E | 0.312 | 0.022 | 0.359 | 0.619 | 0.55678 |
| 789 | F | B | 0.087 | 0.021 | 0.451 | 0.528 | 0.54833 |
| 790 | S | E | 0.333 | 0.022 | 0.359 | 0.619 | 0.54411 |
| 791 | Q | B | 0.261 | 0.022 | 0.359 | 0.619 | 0.54778 |
| 792 | I | B | 0.107 | 0.021 | 0.451 | 0.528 | 0.55589 |
| 793 | L | B | 0.169 | 0.022 | 0.359 | 0.619 | 0.56489 |
| 794 | P | E | 0.319 | 0.019 | 0.141 | 0.84 | 0.59156 |
| 795 | O | E | 0.486 | 0.018 | 0.047 | 0.935 | 0.61078 |
| 796 | P | E | 0.467 | 0.018 | 0.019 | 0.964 | 0.61300 |
| 797 | S | E | 0.484 | 0.018 | 0.047 | 0.935 | 0.62111 |
| 798 | K | E | 0.441 | 0.018 | 0.047 | 0.935 | 0.61456 |
| 799 | P | E | 0.406 | 0.053 | 0.043 | 0.903 | 0.59856 |
| 800 | S | E | 0.525 | 0.053 | 0.043 | 0.903 | 0.57011 |
| 801 | K | E | 0.293 | 0.191 | 0.086 | 0.723 | 0.53356 |
| 802 | R | B | 0.177 | 0.307 | 0.165 | 0.527 | 0.49856 |
| 803 | S | B | 0.138 | 0.321 | 0.252 | 0.427 | 0.47244 |
| 804 | F | B | 0.092 | 0.453 | 0.248 | 0.299 | 0.44833 |
| 805 | I | B | 0.052 | 0.578 | 0.229 | 0.194 | 0.42511 |
| 806 | E | B | 0.174 | 0.538 | 0.173 | 0.289 | 0.41711 |
| 807 | D | B | 0.271 | 0.502 | 0.102 | 0.396 | 0.42489 |
| 808 | L | B | 0.127 | 0.502 | 0.102 | 0.396 | 0.42433 |
| 809 | L | B | 0.11 | 0.385 | 0.097 | 0.517 | 0.42278 |
| 810 | F | B | 0.157 | 0.307 | 0.165 | 0.527 | 0.44522 |
| 811 | N | E | 0.514 | 0.191 | 0.086 | 0.723 | 0.45000 |
| 812 | K | E | 0.352 | 0.125 | 0.227 | 0.648 | 0.46144 |
| 813 | V | B | 0.09 | 0.159 | 0.516 | 0.325 | 0.47522 |
| 814 | T | E | 0.377 | 0.087 | 0.683 | 0.23 | 0.49078 |
| 815 | L | B | 0.239 | 0.079 | 0.992 | 0.329 | 0.50433 |
| 816 | A | B | 0.28 | 0.019 | 0.141 | 0.84 | 0.50867 |
| 817 | D | E | 0.485 | 0.018 | 0.047 | 0.935 | 0.51411 |
| 818 | A | B | 0.263 | 0.018 | 0.019 | 0.964 | 0.53700 |
| 819 | G | E | 0.397 | 0.058 | 0.017 | 0.925 | 0.54322 |
| 820 | F | B | 0.27 | 0.268 | 0.043 | 0.689 | 0.55489 |
| 821 | I | B | 0.192 | 0.622 | 0.015 | 0.363 | 0.55344 |
| 822 | K | E | 0.701 | 0.717 | 0.014 | 0.269 | 0.54511 |
| 823 | Q | E | 0.38 | 0.717 | 0.014 | 0.269 | 0.52911 |
| 824 | Y | B | 0.121 | 0.717 | 0.014 | 0.269 | 0.52900 |
| 825 | G | E | 0.511 | 0.717 | 0.014 | 0.269 | 0.52711 |
| 826 | D | E | 0.606 | 0.622 | 0.015 | 0.363 | 0.52833 |
| 827 | C | B | 0.061 | 0.43 | 0.016 | 0.555 | 0.52756 |
| 828 | L | E | 0.358 | 0.181 | 0.016 | 0.803 | 0.52267 |
| 829 | G | E | 0.648 | 0.058 | 0.017 | 0.925 | 0.50356 |
| 830 | D | E | 0.513 | 0.058 | 0.017 | 0.925 | 0.50156 |
| 831 | I | E | 0.44 | 0.257 | 0.016 | 0.727 | 0.49167 |
| 832 | A | E | 0.316 | 0.354 | 0.048 | 0.598 | 0.47578 |
| 833 | A | B | 0.057 | 0.455 | 0.046 | 0.498 | 0.44889 |
| 834 | R | E | 0.279 | 0.502 | 0.102 | 0.396 | 0.41822 |
| 835 | D | B | 0.229 | 0.502 | 0.102 | 0.396 | 0.38722 |
| 836 | L | B | 0.065 | 0.538 | 0.173 | 0.289 | 0.37256 |
| 837 | I | B | 0.102 | 0.578 | 0.229 | 0.194 | 0.36889 |
| 838 | C | B | 0.045 | 0.578 | 0.229 | 0.194 | 0.38056 |
| 839 | A | B | 0.064 | 0.453 | 0.248 | 0.299 | 0.37622 |
| 840 | Q | B | 0.255 | 0.478 | 0.309 | 0.214 | 0.36656 |
| 841 | K | E | 0.289 | 0.231 | 0.33 | 0.439 | 0.36333 |
| 842 | F | B | 0.136 | 0.125 | 0.227 | 0.648 | 0.35889 |
| 843 | N | B | 0.261 | 0.052 | 0.084 | 0.864 | 0.35533 |
| 844 | G | B | 0.107 | 0.02 | 0.205 | 0.775 | 0.35711 |
| 845 | L | B | 0.076 | 0.023 | 0.655 | 0.322 | 0.34956 |
| 846 | T | B | 0.09 | 0.021 | 0.756 | 0.223 | 0.35111 |
| 847 | V | B | 0.05 | 0.021 | 0.756 | 0.223 | 0.35578 |
| 848 | L | B | 0.063 | 0.021 | 0.451 | 0.528 | 0.35978 |
| 849 | P | B | 0.099 | 0.019 | 0.141 | 0.84 | 0.37844 |
| 850 | P | B | 0.187 | 0.018 | 0.088 | 0.893 | 0.41011 |
| 851 | L | B | 0.16 | 0.019 | 0.141 | 0.84 | 0.42733 |
| 852 | L | B | 0.122 | 0.019 | 0.141 | 0.84 | 0.44978 |
| 853 | T | E | 0.283 | 0.053 | 0.043 | 0.903 | 0.46611 |
| 854 | D | E | 0.463 | 0.782 | 0.003 | 0.216 | 0.46478 |
| 855 | E | E | 0.516 | 0.782 | 0.003 | 0.216 | 0.44367 |
| 856 | M | B | 0.189 | 0.858 | 0.002 | 0.139 | 0.41467 |
| 857 | I | B | 0.103 | 0.923 | 0.002 | 0.076 | 0.38522 |

2-In silica prediction and selection of candidate immunogenic epitopes:

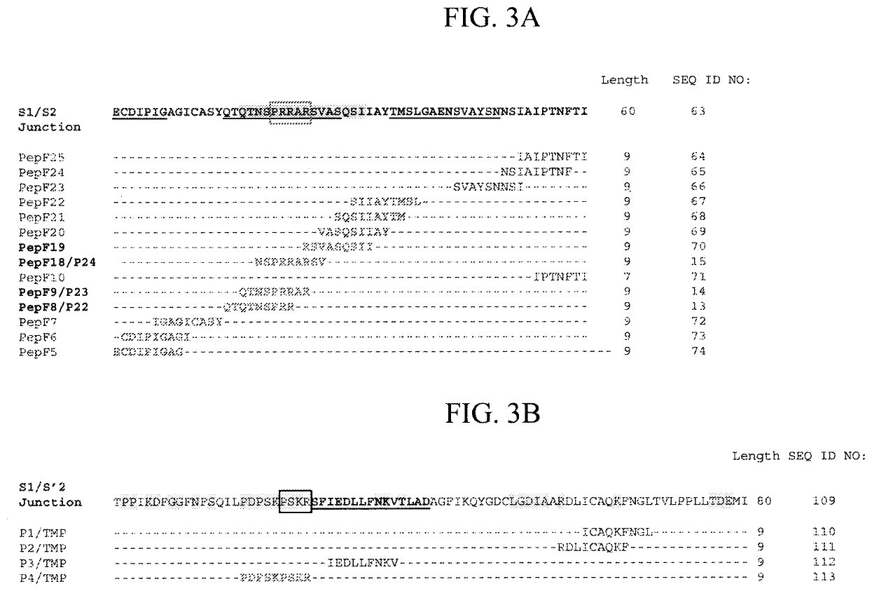

FIG. 3A—shows fourteen peptides predicted to be generated bye antigen presenting cells (APC) Proteasome/Immunoproteasome's upon the processing of the SARS-COV-2 S protein S1/S2 junction sequence. Interestingly, the sequences of seven peptides PepF8/P22, PepF9/P23, PepF18/P24, PepF19, PepF20, PepF21 and PepF22 (FIG. 3), appear, overlaps or surround the solvent exposed sequence Q662 to S676 (Table 3), four peptides PepE8/P22, PepF9/P23, PepF18/P24, PepF19 display or overlap with the PRRAR (SEQ ID NO:107) motif. In addition, the sequences of the antigenic peptides PepF21 and PepF22 overlaps the solvent exposed sequence spanning residues T683 to N696 (FIG. 3), The sequences of the antigenic peptides Pepf25, PepF6 and PepF7 also appears in or overlaps the solvent exposed sequence spanning residues E648 to H654 (FIG. 3). Meanwhile FIG. 3B shows four peptides predicted to be generated by antigen presenting, cells (APC) Proteasomellmmunoproteasome's upon the processing of the SARS-CoV-2 S protein S1/S′2 junction sequence. Peptide P4/TMP (SEQ ID NO: 113) overlaps with the TMPRSS2 cut site.

Based on these observations, the sequences at the spike protein S1/S2 and S1/S′2 junction spanning the 14 (S1/S2) and the 4 (S1/S′2) peptides were analyzed to design multivalent protein subunits for vaccination against SARS-CoV-2. [See results section 8]

3-Design of polypeptides for the of the humoral immunity to SARS-CoV-2.

To investigate whether the predicted peptides from the SARS-Cov-2 S1/S2 junction region are effectively targeted by the COVID19 patient's humoral immunity, linear homo-polymerization was used to design a set of polypeptides using two core sequences of 16 and 10 residues that encompasses the sequences of the predicted antigenic peptides. These peptides were designed to include repetitions. of the core sequences that span the PRRAR (SEQ ID NO: 107) furin cut site (FCS) motif [Table 4]. The previously determined antigenic peptides and the designed polypeptides were used to monitor the cohort of 500 PCR+ SARS-CoV-2 patients for specific antibody response. Furthermore, these peptides/polypeptides can be structurally modified to introduce subtle conformational changes, including modifications of WIC-anchor substitutions, which increase their stability, protease resistance and immunogenicity and enhance their vaccinating potential.

TABLE 4
Design and Sequence of a set of peptides with a core sequence of 16 amino
acid containing the FCS motif PRRA (peptide P1 to P5) and a shorter sequence of 10 amino
acids including the core PRRA sequence (peptides P19 & P21).
Peptides P2, P3 and P21 were designed by linear polymerization. They display
respectively two and three repetitions of the core peptide motif. To allow for a good
and independent folding of the core sequence in these 3 peptides, we added a GGGS linker
sequence to the NH₂ terminal of the peptides sequences. We also added 2 cysteine
residues C respectively to the NH and COOH terminus of the peptide sequence in peptides
P4 and P5 to create a disulfide bridge and for cyclization of the peptide. We also
added an NH₂ Biotin Tag (*) to allow antigen binding to the streptavidin-coated plate
used in the indirect ELISA assay.
Peptides P1, P2, P3, P4/TMP are from the S1/S′2 junction region. Underlined sequences
show residues of SARS-CoV-2 S protein located at the junction of S1/S2 These specific
sequences contain epitopes surrounding and/or overlapping the furin proteolytic cut
cite PRRAR.

| | | # Core | |
|---|---|---|---|
| | | Sequence | |
| Peptide Name | Peptide Sequence | Repeats | SEQ ID NO: |
| P1 | *GGGGSQTNSPRRARSVASQSI | 1 | 1 |
| P2 | *GGGGSQTNSPRRARSVASQSIGGGGSQTNSPRRARS | 2 | 2 |
| | VASQSI | | |
| P3 | *GGGGSQTNSPRRARSVASQSIGGGGSQTNSPRRARS | 3 | 3 |
| | VASQSIGGGGSQTNSPRRARSVASQSI | | |
| P4 | *GGGGSQCNSPRRARSVASQCI | 1 | 4 |
| P5 | *GGGGSQCNSPRRARSVASQCIGGGGS* | 1 | 5 |
| P19 | *GGGGSSPRRARSVAS | 1 | 6 |
| P21 | *GGGGSSPRRARSVASGGGGSSPRRARSVASGGGGSS | 3 | 7 |
| | PRRARSVAS | | |
| F22 | *GGGGSQTQTNSPRR | 1 | 8 |
| P23 | *GGGGSQTNSPRRAR | 1 | 9 |
| 224 | *GGGGSNSPRRARSV | 1 | 10 |
| P4/TMP | PDPSKPSKR | 1 | 113 |
| P4/TMP bn | *GGGGSPDPSKPSKR | 1 | 115 |
| P4/TMP- | GGGGSPDPSKPSKRGGGGSPDPSKPSKRGGGGSPDPS | 3 | 116 |
| Pol3 | KPSKR | | |
| P4/TMP- | *GGGGSPDPSKPSKRGGGGSPDPSKPSKRGGGGSPDP | 3 | 117 |
| Pol3-bn | SKPSKR | | |
| P3/TMP | IEDLLFNKV | 1 | 112 |
| P3/TMP- | *GGGGSIEDLLFNKV | 1 | 118 |
| bn | | | |
| P2/TMP | RDLICAQKF | 1 | 111 |
| P2/TM- | *GGGSRDLICAQKF | 1 | 119 |
| bn | | | |
| P1/TMP | ICAQKFNGL | 1 | 110 |
| P1/TMP- | *GGGGSICAQKFNGL | 1 | 120 |
| bn | | | |

The GGGGS residues are underlined. These are used as linkers to engineer peptides/polypeptides where epitopes retain their to structures and immunogenicities.

Developinent of an indirect ELISA assay: A standard indirect ELISA assay was designed for the detection of specific anti-SARS-CoV-2 laG antibodies in human serum samples from 492 people tested positive to SAR-CoV-2 using a PCR assay. This group included asymptomatic individuals and others mildly, moderately and severely covid19 affected people. For this assay, as target antigens the engineered peptides P1 to P21 [see Table 4] and the peptides from the S protein S1/S2 junction [P22, P23 and P24] predicted to in be generated. by the proteasome/immunoproteasome of an antigen-presenting cell of an affected person and presented to the effector immune cell as antigenic determinants were used. The synthetiad peptides were used with added Biotin to their termini and 96-microwell plates coated with purified streptavidin for this indirect ELISA assay. Checkerboard titration curves of serum samples from 50 SARS-CoV-2 with COVID-19 PCR (+) individuals and 100 pre-pandemic human serum samples led to the determination that a dilution of 1:100 was of optimal sensitivity and specificity, and then this dilution was used in the serological study. The Optical Density (OD) threshold value was set at 0.450 [FIG. 4A]. Peptide P3 (Table 4) gave the best signal-to-noise ratio thus it was used for testing reference serum samples and sera from the large cohort. No cross-reaction of the secondary antibody was observed and the ELISA assay was highly specific. The plates coating and ELISA protocols are detailed in Material and Methods section 7 above (see specifically 7.b.-7.c.).

5-Serum Samples testing

5.1. Testing of reference sera: A set of naïve sera (n=100) from health individuals collected before the SARS-CoV-2 pandemic, was tested to establish the cut off for the ELISA test: developed with the two sets of specific SARS-CoV-2 antigens (FIG. 4). Testing of the WHO set of reference sera acquired from the National Institute for Biological Standards and Control, UK gave unexpected results. Indeed, as Shown in FIG. 4B, out. of the 14 presumably negative sera, 9 (64%) were positive. Interestingly, the control negative sera collected during the peak of the pandemic from local healthy SARS-CoV-2 supposedly naive individual showed more than 80% of positive sera (FIG. 4C). Moreover, out of the 23 presumably positive sera provided by the NIBSC it was determined that 60% environ were negative (FIG. 4D), Since no data on the clinical status and/or the results of PCR testing of the individuals from whom the reference sera were available, these unexpected data are considered later in light of the analysis of the results generated from the study of the large cohort.

5.2. Testing of the Cohort: Testing of the cohort of 500 SARS-CoV-2/PCR (+) people, showed that a high percentage of the patients developed antibodies to the epitopes present respectively in the P3 (80%, FIG. 5A), P4/TMP-Pol3 (86%, FIG. 5B), P3L (88%, FIG. 5C) and SJ/FT (97%, FIG. 5D) sequences derived from the SARS-CoV-2 S protein S1/S2 subunits junction.

6-Correlation of antibody response COVID9 patients with a protective immunity SARS-CoV-2,

Out of the 500 PCR-confirmed COVID-19 patients enrolled in this study 63% were asymptomatic while 25 developed the mild form of the disease. FIG. 6 shows a dot plot produced using R and ggplot2 (v 3.3.3) library, This chart presents the distribution of “P3 OD” versus COVID19 disease status in a cohort of 492 SARS-CoV-2 infected individuals, The binwidth used in this chart is 0.05. The p-value given by the Fisher's exact test is p<0.001. So, the null hypothesis is rejected, These data show a highly significant difference between patients, in term of antibody, response to P3, based on their “Disease status”. There is a strong association between the immune (+) “Immunity status” to antigenic determinants displayed in the P3 polypeptide and the asymptomatic and mild forms of the disease “Disease status”.

7-Immunization Balbe Mice with the P3 Peptides:

FIG. 7A shows the results of the ELISA assay of the sera obtained 6 weeks following mice immunization with 200 μg of polypeptide P3, designed by epitope homopolymerization to increase immunogenicity. Polypeptide P3 gave a high antibody response. The antibody response is higher in the group of mice fin which we used the CpG TLR9 ligand adjuvant. Use of other adjuvants, including alum, Toll-like receptor agonists and oil-in-water emulsions can alternatively be considered. Combination adjuvants such as alum/TLR9 agonists or CpG ODNs with MDF MPLA can be tested (Invivogen 2021, hypertext transfer protocol secure://www.invivogen.com/vaccine-adjuvants. Nanishi, et al, 2020).

8-Design of multi-epitope subunits for immununization/vaccination. The inventors analyzed a number of sequences of various lengths spanning the junction of the SARS-CoV-2 S protein S1/S2 subunit junction spanning the furin and TNIPRSS2 cut sites and containing the sequences of the 18 (14+3) predicted epitopes. We engineered protein subunits with favor able amino acids for proper antigen folding and selected those with the best predicted physiological properties of utmost importance for the development of subunit vaccines namely the antigenicity, allergenicity and stability. The designed seven subunits were (PH, to SEQ ID 44), (SJ-80 SEQ ID 52), (SJ-100, SEQ ID 56),(SJ-120 SEQ ID 60), (RIFT SEQ ID 144). (SJ-S2′/FP1, SEQ ID 148), (SJ-S2′/FP1-2 SEQ ID 153) included the SARS-Cov-2 furin cut site motif, PRRAR (SEQ ID NO: 107), the TMPRSS2 cut site motif, the Fusion Protein1 FP1 and /or the Fusion Protein2 EFP21 protein sequences and encompassed the sequences of the 18 antigenic peptides potentially generated by the APC Proteasome/immunoproteasome. Among these peptides are the ones that elicit the protective antibody response in COVID19 patients.

FIGS. 19-30 show the design of these vaccine subunits and the sequences of their corresponding codon-optimized cDNA along with their corresponding amino acid sequences. The sequence coding of the human β defensin 1 was added to increase the stability and antigenicity of the original sequence. Table 5 shows the predicted physiological properties of the selected multi-epitope subunit vaccines. The antigenic multi-epitopes sequence we developed are suitable for various vaccine delivery technologies.

TABLE 5
Predicted physiochemical properties of the polypeptides/protein
subunits from the S1/S2/S’2 subunits Junction sequence.
The lower the instability index II the more stable is the protein.
The lower is the GRAVY score the more soluble is the protein.

| | SJ-PT | SJ-S2’/PP1 | SJ-S2’/PP1-2 | P3-L | SJ-80 | SJ-100 | SJ-120 |
|---|---|---|---|---|---|---|---|
| Number | 230 | 140 | 220 | 107 | 80 | 100 | 120 |
| of amino | | | | | | | |
| acids | | | | | | | |
| Molecular | 24835.29 | 15203.52 | 23811.32 | 11105.12 | | 10682.89 | 12820.20 |
| Weight | | | | | | | |
| Theoretical | 4.85 | 5.28 | 4.78 | 10.55 | 6.74 | 7.2 | 5.46 |
| PI: | | | | | | | |
| Number | 20 | 13 | 18 | 3 | 4 | 5 | 7 |
| of | | | | | | | |
| negatively | | | | | | | |
| charged | | | | | | | |
| residues | | | | | | | |
| (Asp + | | | | | | | |
| Glu) | | | | | | | |
| Number | 16 | 12 | 14 | 9 | 4 | 5 | 5 |
| of | | | | | | | |
| positively | | | | | | | |
| charged | | | | | | | |
| residues | | | | | | | |
| (Arg + | | | | | | | |
| Lys): | | | | | | | |
| Formula | C₁₀₈₉H₁₇₃₇N₂₈₃ | C₆₉₂H₁₀₈₆ | C₁₀₆₇H₁₆₇₂ | C₂₃₉H₇₄₇ | C₃₆₄H₅₇₆ | C₄₆₃H₇₂₅ | C₃₈₂H₈₆₅ |
| | O₃₄₆S₁₂ | N₁₇₄O₂₀₂S₄ | N₂₅₆O₃₂₅S₁₂ | N₁₄₉O₁₆₄S₄ | N₁₀₆O₁₂₃S₄ | N₁₃₃O₁₅₀S₄ | N₁₅₈O₁₃₄S₅ |
| Estimated | 1 hour | 7.2 hours | 1.9 hour | 30 hours | 1.4 hours | >20 hours | 30 hours |
| half-life | (mammalian | (mammalian | (mammalian | (mammalian | (mammalian | (mammalian | (mammalian |
| | reticulocytes, | reticulocytes, | reticulocytes, | reticulocytes, | reticulocytes, | reticulocytes, | reticulocytes, |
| | in vitro) | in vitro) | in vitro) | in vitro) | in vitro) | in vitro) | in vitro) |
| | >30 min (yeast, | >20 hours | >20 hours | >20 hours | 3 min hours | >20 hours | >20 hours |
| | in vivo). | (yeast, in vivo). | (yeast, in vivo). | (yeast, in vivo). | (yeast, in vivo). | (yeast, in vivo). | (yeast, in vivo). |
| | >10 hours | >10 hours | >10 hours | >10 hours | >10 hours | ? hours | >10 hours |
| | (Escherichia | (Escherichia | (Escherichia | (Escherichia | (Escherichia | (Escherichia | (Escherichia |
| | coli, in vivo). | coli, in vivo). | coli, in vivo). | coli, in vivo). | coli, in vivo). | coli, in vivo). | coli, in vivo). |
| Instability | 41.86 | 31.41 | 33.45 | 72.71 | 36.29 | 30.23 | 30.23 |
| index(II) | | | | | | | |
| Aliphatic | 87.43 | 93.50 | 90.09 | 34.95 | 82.62 | 80.10 | 80.50 |
| index | | | | | | | |
| Average | 0.050 | 0.073 | 0.16 | −0.535 | −0.044 | −0.074 | −0.097 |
| of | | | | | | | |
| hydro- | | | | | | | |
| pathicity | | | | | | | |
| (GRAVY) | | | | | | | |

9-Modeling of the Structure of Selected Peptides:

Ab intio 3D models of the short (10 AA), medium (16AA) and long (32AA) peptides core sequence and the 3 repeats hornopolymerised version were generated. FIG. 9 shows the 3D models of selected peptides core sequence that: encompasses the FCS epitope. The predicted structural models show an Alpha Coilinelix fold that is consistent with the structure of immunogenic epitopes and. solvent accessible protein sequence. A repeated display of the Alpha Coil/Helix fold is Observed in the .P3 and P3L polypeptides, which show that the 16 and 32 residues peptide sequences containing the PRRAR (SEQ ID NO: 107) epitope folds independently from the G₄S linker. However, the Model predicted for the peptides that have a shorter 10 residues encompassing the FCS epitope [peptide P21] do not display the Alpha Coil/Helix fold. This observation explains the negative data obtained when using P21 in an ELISA assay to monitor the antibody response of the cohort of SARS-CoV-2 infected people data not shown). in addition, the 3D models can he used to design specific drugs that block the SARS-CoV-2 S protein furin cut site. These models and the sequences used to build them up are also useful to generate high affinity specific antibodies and all their derived forms (Fab, ScFv and others IgG fragments) designed to neutralize SARS-CoV-2 S protein furin cut site PRRAR sequence (SEQ ID NO: 107).

10-Prediction of the antigenicity, and allergenicity of the Sars-CoV-2 S protein derived submit.

To check the potential of the polypeptides and the designed subunits to induce a humor al and/or a cellular response against their targeted epitopes, the probability of their antigenicity was estimated using the VaxiJen V 2 O. computational platform. The predicted to scores are shown in Table 6. The data show that the designed polypeptides and subunits are predicted to be antigenic with the subunits having .slightly higher antigenic probability scores. All the designed sequences are non-allergenic.

Table 6 Predicted antigenicity and allergenicity of the developed SARS-CoV-2 subunits vacine. In Table 6A are displayed the predicted antigenicity and in Table 6B the allergenicity, of each multi-epitopes subunit.

TABLE 6A

| Subunit | P3 | P3-L | SJ-80 | SJ-100 | SJ-120 | SJ-PT | SJ-S2’/PP1 | SJ-S2’/PP1-2 |
|---|---|---|---|---|---|---|---|---|
| VAXiJEN | 0.4331 | 0.4436 | 0.6397 | 0.6254 | 0.6292 | 0.4512 | 0.3458 | 0.4882 |

TABLE 6B

| Subunit | P3 | P3-L | SJ-80 | SJ-100 | SJ-120 | SJS1/S2/S2’PT | SJ-S2’/PP1 | SJ-S2’/PP1-2 |
|---|---|---|---|---|---|---|---|---|
| AllerTOP | (−) | (+) | (+) | (−) | (+) | (−) | (−) | (−) |
| Algpred | (−)** | (−)** | (−) ® | (−)** | (−)** | (−) ®* | (−)* | (−) ®* |
| | 0.5130 | 0.7027 | 0.4150 | 0.5764 | 1.0501 | 0.5310 | −0.0665 | −0.34112712 |

# Prediction by SVM method basec on amino acid composition [Score Threshold = −0.4]
®Prediction based on SVM method based on dipeptide composition [Score Threshold = −0.2]
*The protein sequence does not contain experimentally proven IgE epitope.

11-Determination of the binding of antigenic peptides to-MHX molecules:

At least nine out of the fourteen peptides identified in the SARS-COV-2 S protein S1/S2 subunit junction displaying antigenic determinants that elicited an antibody response associated with protective immunity in COVID 19 patients, are predicted to bind. Class I and class II MHC molecules.

As shown in Table 7 these peptides hind a large array of WIC Class I and Class II molecules. The peptides and peptide products described herein may be contacted with cells expressing an MTC molecule capable of presenting it to the cellular immune system such as those correlations between peptide and MHC background described in Table 7.

According to the Net:MHCpan 2.3 Server that uses artificial neural networks (ANNs), the binding, level to MTC class I molecules was predicted to be strong (SB) for 3 antigenic peptides displaying the PRARR furin cut site motif MLA-B*08:01, MA-B*07:02) and the adjacent peptide SVASOSIIAY (SEQ ID NO: 25) to HLA-A*26:01 and 15:01. Table 7: Predicted Binding of the antigenic peptides vanning the SARS-CoV2 S protein S1/S2/S′2 function to MHC molecules. Al-MHC Class1 Junction S1/S2 peptides binding prediction using the artificial neural networks (ANNs). The peptides predicted to display a strong binding (SR) are shown underlined. A2-A3 MHC Class1 and. MHC Class II alleles predicted by the Propred server to bind the antigenic peptides encompassing or in the vicinity of the S protein furin cleavage site. A4-MTC Class II alleles predicted to bind strongly to antigenic peptides from the S1/S′2 junction region. Peptides encompassing of in the Vicinity of the TMPRSS2 cleavage site and Fusion peptide FPI are highlighted.

TABLE 7 A1

[symbol]

[symbol]

indicates data missing or illegible when filed

TABLE 7A2

| PEPTIDE | MHC Class I ALLELES |
|---|---|
| CDIPIGAGI | HLA-A1 HLA-A2 HLA-A*0201 HLA-A*0205 HLA-A*1101 HLA-A24 HLA-A3 HLA-A*3101 HLA-A*3302 |
| (SEQ ID NO: 29) | HLA-A68.1 HLA-A20 Ca HLA-A2.1 HLA-B14 HLA-B*2702 HLA-B*2705 HLA-B*3501 HLA-B*3701 |
| | HLA-B*3801 HLA-B*3901 HLA-B*3902 HLA-B40 HLA-B*4403 HLA-B*5101 HLA-B*5102 HLA- |
| | B*5103 HLA-B*5201 HLA-B*5301 HLA-B*5401 HLA-B*51 HLA-B*5801 HLA-B60 HLA-B61 HLA-B62 |
| | HLA-B7 HLA-B*0702 HLA-B8 HLA-Cw*030 HLA-Cw*040 HLA-Cw*060 HLA-Cw*070 MHC-Db MHC- |
| | Db rev MHC-Dd MHC-Kb MHC-Kd MHC-Kk MHC-Ld |
| ECDIPIGAG | HLA-A1 HLA-A2 HLA-A*0201 HLA-A*0205 HLA-A*1101 HLA-A24 HLA-A3 HLA-A*3101 HLA-A*3302 |
| (SEQ ID NO: 30) | HLA-A68.1 HLA-A20 Ca HLA-A2.1 HLA-B14 HLA-B*2702 HLA-B*2705 HLA-B*3501 HLA-B*3701 |
| | HLA-B*3801 HLA-B*3901 HLA-B*3902 HLA-B40 HLA-B*4403 HLA-B*5101 HLA-B*5102 HLA- |
| | B*5103 HLA-B*5201 HLA-B*5301 HLA-B*5401 HLA-B*51 HLA-B*5801 HLA-B60 HLA-B61 HLA-B62 |
| | HLA-B7 HLA-B*0702 HLA-B8 HLA-Cw*030 HLA-Cw*040 HLA-Cw*060 HLA-Cw*070 MHC-Db MHC- |
| | Db rev MHC-Dd MHC-Kb MHC-Kd MHC-Kk MHC-Ld |
| IGAGICASY | HLA-A1 HLA-A2 HLA-A*0201 HLA-A*0205 HLA-A*1101 HLA-A24 HLA-A3 HLA-A*3101 HLA-A*3302 |
| (SEQ ID NO: 28) | HLA-A68.1 HLA-A20 Ca HLA-A2.1 HLA-B14 HLA-B*2702 HLA-B*2705 HLA-B*3501 HLA-B*3701 |
| | HLA-B*3801 HLA-B*3901 HLA-B*3902 HLA-B40 HLA-B*4403 HLA-B*5101 HLA-B*5102 HLA- |
| | B*5103 HLA-B*5201 HLA-B*5301 HLA-B*5401 HLA-B*51 HLA-B*5801 HLA-B60 HLA-B61 HLA-B62 |
| | HLA-B7 HLA-B*0702 HLA-B8 HLA-Cw*030 HLA-Cw*040 HLA-Cw*060 HLA-Cw*070 MHC-Db MHC- |
| | Db rev MHC-Dd MHC-Kb MHC-Kd MHC-Kk MHC-Ld |
| QTQTNSPRR | HLA-A1 HLA-A2 HLA-A*0201 HLA-A*0205 HLA-A*1101 HLA-A24 HLA-A3 HLA-A*3302 HLA-A68.1 |
| (SEQ ID NO: 13) | HLA-A20 Ca HLA-A2.1 HLA-B14 HLA-B*2702 HLA-B*2705 HLA-B*3501 HLA-B*3701 HLA-B*3801 |
| | HLA-B*3901 HLA-B*3902 HLA-B40 HLA-B*4403 HLA-B*5101 HLA-B*5102 HLA-B*5103 HLA- |
| | B*5201 HLA-B*5301 HLA-B*5401 HLA-B*51 HLA-B*5801 HLA-B60 HLA-B61 HLA-B62 HLA-B7 HLA- |
| | B*0702 HLA-B8 HLA-Cw*030 HLA-Cw*040 HLA-Cw*060 HLA-Cw*070 MHC-Db rev MHC-Dd MHC- |
| | Kb MHC-Kd MHC-Kk MHC-Ld |

Table 7 A3

| PEPTIDE | MHC Class II ALLELES |
|---|---|
| IGAGICASY | HLA-DQA10301-DQB10301 HLA-DQA10501-DQB10303 HLA-DQA10301-DQB10301 |
| (SEQ ID NO: 28) | HLA-DQA10501-DQB10303 HLA-DQA10501-DQB10301 HLA-DQA10501-DQB10301 |
| QTNSPRRAR | HLA-DQA10201-DQB10402 HLA-DQA10303-DQB10402 HLA-DQA10201-DQB10402 |
| (SEQ ID NO: 14) | HLA-DQA10303-DQB10402 HLA-DQA10201-DQB10402 HLA-DQA10303-DQB10402 |
| | HLA-DQA10201-DQB10402 HLA-DQA10303-DQB10402 HLA-DQA10303-DQB10402 |
| | HLA-DQA10303-DQB10402 HLA-DQA10201-DQB10402 HLA-DQA10201-DQB10402 |
| | HLA-DQA10201-DQB10402 HLA-DQA10303-DQB10402 HLA-DQA10201-DQB10402 |
| | HLA-DQA10303-DQB10402 HLA-DQA10201-DQB10402 HLA-DQA10303-DQB10402 |
| | HLA-DQA10201-DQB10402 HLA-DQA10303-DQB10402 HLA-DQA10303-DQB10402 |
| | HLA-DQA10303-DQB10402 HLA-DQA10201-DQB10402 HLA-DQA10201-DQB10402 |
| RSVASQSH | H-2-1As H-2-1As HLA-DQA10103-DQB10603 HLA-DQA10201-DQB10402 HLA- |
| (SEQ ID NO: 26) | DQA10103-DQB10603 HLA-DQA10201-DQB10402 H-2-1As HLA-DQA10103- |
| | DQB10603 HLA-DQA10103-DQB10603 H-2-1As HLA-DQA10103-DQB10603 HLA- |
| | DQA10103-DQB10603 H-2-1As HLA-DPA10201-DPB11401 HLA-DPA10201-DPB11401 |
| | HLA-DQA10103-DQB10603 HLA-DQA1020-DQB10402 HLA-DPA10201-DPB11401 |
| | HLA-DQA10103-DQB10603 HLA-DQA10201-DQB10402 HLA-DQA10103-DQB10603 |
| | HLA-DQA10103-DQB10603 H-2-1As HLA-DPA10201-DPB11401 HLA-DPA10201- |
| | DPB11401 HLA-DPA10201-DPB11401 |
| SQSHAYTM | HLA-DQA10501-DQB10303 HLA-DQA10501-DQB10303 HLA-DQA10201-DQB10202 |
| (SEQ ID NO: 23) | HLA-DQA10201-DQB10202 |
| VASQSHAY | DRB1_1201 DRB1_1201 DRB1_1201 DRB1_1201 DRB1_1201 DRB1_1201 DRB1_1201 |
| (SEQ ID NO: 24) | DRB1_1201 |

TABLE 7 A4

[symbol]

[symbol]

indicates data missing or illegible when filed

Underlined entries in Table 7A4 correspond to antigenic epitopes that are located in or overlap with the TMPRSS2 cut site in the selected polypeptides and are useful for aenerating antibodies that can interfere with the virus's infectivity and thus provide protective immunity

Numerous modifications and variations on the present invention are possible in light of the above teachings. It is, therefore, to be understood that within the scope of the accompanying claims, the invention may be practiced otherwise than as specifically described herein,

### REFERENCES

Albini, A., Calabrone, L., Carlini, V., Benedetto, N., Lombardo, M., Bruno, A., & Noonan, D. M. (2021). Preliminary Evidence for IL-10-Induced ACE2 mRNA Expression in Lung-Derived and Endothelial Cells: implications for SARS-Cov-2 ARDS Pathogenesis. Front Immunol, 12,7.18136 doi:10.339/fintinu.2021.718136

Albini, A., Di Guardo, G., Noonan, D. M., & Lombardo, M. (2020). The SARS-COV-2 receptor. ACE-2, is expressed on many different cell types: implications for ACE-inhibitor- and angiotensin II receptor blocker ba cardiovascular therapies, Intern Emerg Med. 15(5), 759-766. doi: 10,1007/s11739-020-02364-6

Angeli, F., Spanevello, A Rebohli, G., Visca, D., & Verclecchia, P. (2021). SARS-CoV-2 vaccines: Lights and shadows. Eur J Intern Med, 88, 1-8. doi:10.1016j.cjim.2021.04.019

Belouzard, S., Chu, V. C., & Whittaker, G. R. (2009). Activation of the SARS coronavints spike protein via sequential proteolytic cleavage at two distinct sites, Proc Natl Acad Sci U S A, 106(14), 5871-5876. doi:10.1073/pnas.0809524106

Belotizard, S., Millet, J. K., Licitra, B, N., & Whittaker, G. R, (2012). Mechanisms of coronavirus cell entry mediated by the viral spike protein Viruses; 4(6), 1011-1033. doi:10.3390/v4061011

Bestle, D., Heindl, M. R., Limbuti, H., Van Larn van. T., Pilararn, O., Moulton, H., Bottcher-Friebertshatiser, E. (2020). TMPR 882 and rural are both essential for proteolytic activation of SARS-COV-2 in human airway cells, Life Sci Alliance, 3(9), doi:10.26508/lsa,202000786

Bloch. E. M., Shoham, S., Casadevall, A., Sachais, B. S., Shaz., B., Winters, J. L., Tobian, A. A. (2020). Deployment of convalescent plasma for the prevention and treatment of COVID-19. J Clin Invest, 130(6), 2757-2765. doi:10.1172/JC1138745

Bos, R., Rutten, L., van der Lubbe, J. E. M., Bakkers, M. J. G., Harden berg, G., Wegmarm, F., Schultemaker, .H. (2020), Ad26 vector-based COVID-19 vaccine encoding a prefusion-stabilized SARS-CoV-2 Spike immunogen induces potent humoral and cellular immune responses, NPJ Vaccines, 5(1), 91. dot:10.1038/s41541-020-00243-x

Braun, J., Loyal, L., Frentsch, Wendisch, D., Georg, P., Kurth, F., Thiel, A. (2020). SARS-CoV-2-reactive T cells in healthy donors and patients with COVID-19, Nature. 387(7831), 270-274. doi:10.1038/s41586-020-2598-9

Brotons C, Serrano Fernandez D, Garcia-Ramos C, Ichazo B, Lemaire J, et al. Seropnlvalence against COVID-19 and follow-up of suspected cases in primary health care in Spain. medRxiv. 2020:2020.06.13.20130575.

Callow, K. A Parry:, H. F., Sergeant, M., & Tyrtell, D. A. (1990). The time course of the immune response to experimental coronavirus infection of man. Empidemiol Infect, 105(2), 435-446. doi:10,1017/s0950268800048019

Chan, J. F., .Kok, K. H, Zhu, Z., Chu, H., To, K. K., Yuan, S., & Yuen, K. V. (2020). Genomic characterization of the 2019 novel human-pathogenic coronavirus isolated from a patient with atypical pneumonia after visiting Wuhan. Emerg Microbes Infect, 9(1), 22.1-236 doi;10.1080/22221751.2020.1719902

Chen, G., Wu, D., Guo, W., Cao, Y. Huana, D., Wane, H. Nina, Q. (2020). Clinical and immunological features of severe and moderate coronavirus disease 2019. J Clin Invest, 130(5), 2620-2629. doi:10.1172/JC11371137244

Chen, Z , Rum P, Wang, L., Nie. X., ma, X., & Tan, Y. (2021), T and B cell Epitope analysis of SARS-CoV-2 S protein based on imintmoinformaties and experimental research. J Cell Mol Med. 25(2), 1274-1289. doi;10.1111/jcmm 16200

Oxnard, B., C:, del-amballerie, X., Canard, B., Seidah, N. G., & Decroly, E. (2020). The spike glycoprotein of the new coronavirus 2019-nCoV contains a furin-like cleavage site absent in CoV of the same Blade. Antiviral Res, 176, 104742. doi:10.1016/j.antiviral.2020.104742

Crooke, S. N., Ovsyannikova, I. G., Kennedy, R. B., & Poland, G. A. (2020). Immunoinformatic identification of B cell and. I cell epitopes in the SARS-CoV-2 protcome. Sci Rep, 10(1), 14179. doi:10.1038/s41598-020-70864-8

Csistzovszki et at. Corenavirus vaccine. U.S. Pat. No. 10,973,909 B1, Apr. 13, 2021

Dawood, F. S., Ricks, P., Njie, G. Daugherty, M., Davis, W., Fuller, J. A., Bennett, S. D. (2020). Observations of the global epidemiology of COVID-19 from the prepandemic period using web-hosed surveillance; a cross-sectional analysis. Lancet Intfect Dis 20(11), 1253-1262. doi:10,1016/S1473-3099(20)30581-8

Deng, W., Baa, L, Liu J., Xiao. C., Xue. J. Qin C. (2020). Primary exposure to SARS-COV-2 protects against reinfection in rhesus macaques. Science, 369(6505), 818-823. doi:10.1126/scienec.abc5343

Edridee A W, Kaczorowska S M, Hoste A C, Bakker M, Klein M, Jebbink MF, et al. Human coronavirus reinfection dynamics: lessons for SARS-CoV-2. med.Rxiv, 2020:2020.05.11.20086439.

Elflein J. (2020). Coronavirus (COVID-19) disease pandemic-Statistics & Facts [Online], statista. Available: hypertext transfer protocol secure://www.statista.com/topics/5994/the-coronavitos-disease-covid-19-ontbreak/

European Centre for Disease. Prevention and Control. SARS-CoV-2 variants of concern as of 9 Sep., 2021. hypertext transfer protocol secure://www.ecdc.europa.eu/en/covid-19/Variants-concern

Gao, Q., Bao. L., Mao, H., Wang, L., Xu, K., Yang, M., Qin, C. (2020). Development of an inactivated vaccine candidate for SARS-CoV-2. Science, 369(6499), 77-81. doi:10.1126/science.abc1932

Garcia-Baronat, M., Diez-Rivero, C.M., Reinherz, E. L. & Recite, P. A. (2008). PVS: a web server for protein sequence variability analysis tuned to facilitate conserved epitope discovery. Nucleic Acids Res, 36(Web Server issue), W35-41. doi10.1093/nar.gkn211

Ghisolfi, S., Almas, I., Sordefur, J. C., von Carnap, T., Heitner, J., & Bold, T. (2020). Predicted COVID-19 fatality rates based on age, sex, comorbidities and health system capacity. BMJ Glob Health, 5(9). doi:10.1136/bmjgh-2020-003094

GISAID hypertext transfer protocol secure://www.gisaid.org

Greaney, A. J., Starr, T, N., Gilchuk, P., Zost, S. J., Binahtein, E., Loes, A. N., Bloom., J. D. (2021). Complete Mapping. of Mutations to the SARS-COV-2 Spike Receptor-Binding Domain that Escape Antibody Recognition. Cell Host Microbe, 29(1), 44-57 e49. doi:10.1016./j, chom.2020.11.007

Grifoni, A., Weiskopf, D., Ramirez, S. L., Mateus, J., Dan, J. M. Moderbacher, C. R., Sette, A. (2020). Targets of T Celt Responses to SARS-CoV-2 Coronavirus in Humans with COVID-19 Disease and Unexposed Individuals, Cell, 181(7), 1489-1501 e1415. dio:10.1016/j.cell.2020.05.015

Herzog S, De Bic J, Abrams S, Wauters I, Ekinci F., Patteet L, et at. Seroprevalence of IgG antibodies against SARS coronavirus 2 in Belgium: a prospective cross-sectional study of residual samples. medRxiv. 2020:2020.06.08.20123179.

Iverson P. L. & Bavari S. (2021). Inactivated COVID-19 vaccines to make a global impactLancet Infect Dis. 2021 Jun. 21 (6): 746-748. doi: 10.101.6/S1473-3099(21)00020-7

Jackson, L. A., Anderson, E. J . RouphaeL N. G., Roberts, P. C., Makherte, M., Coler, R. N., (2020), An mRNA Vaccine against SARS-CoV-2 : Preliminary Report, N Engl J Med, 383(20), 1920-1931. doi:10.1056/NEJMoa2022483

Jalkanen, P., KolehmaMen, P., Hakkinen, H. K., Hutiunen, M., P, A., Lundberg, R., Julktmen, I. (2021). COVID-19 mRNA vaccine induced antibody responses against three SARS-CoV-2 variants. Nat Commit 12(1), 3991. doi.:10:1.038/s41467-021-242854

Jespersen, M. C., Peters, B., Nielsen, M., & Marcatili, P. (2017), BepiPred2.0: improving sequence-based B-cell epitope prediction using conformational epticipes. Nucleic Acids Res, 45(W1), W24-W29. doi:10.1093/nar/gkx346

Johnson, B. A., Xie, X., Kalveram, B., Lokagamage, K. G., Murnato, A., Zou, J., Menachery, V. D. (2020). Furin Cleavage Site Is Key to SARS-CoV-2 Pathogenesis, doi:10.1101/2020.08.26.268854

Kalita, P., Padhi, A. K., Zhang, K. Y. J., & Tripathi, T. (2020), Design of a peptide-lased subunit vaccine against: novel coronavirus SARS-CoV-2. Microb Pothog, 145, 104236. doi:10.1016/j.micpath, 2020.104236

Karthik K., Senthilkumar T. M. A., Lidhayavel S., Raj G. D. (2020). Role of antibody-dependent enhancement (ADE) in the virulence of SARS-CoV-2 and its mitigation strategies fear the development of vaccines and immunotherapies to counter COVID-19, Hum. Vaccin Immunother. 16(12): 3055-3060

Kevadiya, B. D. Machhi. J., Herskovitz, J. Oleynikov, M. .D., Blomberg, W, R., Bajwa, N., Gendelman, H. E. (2021). Diagnostics for SARS-CoV-2 infections. Nat Mater, 20(5), 593-605. dot. 10.1038;s41563-020-0090-z

Krause, P. R., Fleming, T. R., Longini, I, M., Peto, R., Briand S., Heymann, D, L., Henao-Restrepo, A. M. (2021). SARS-CoV-2 Variants and Vaccines. N Engl J Med, 385(2), 179-186 doi:10.1056/NEJMsr2105280

Kremer, E. J. (2020). Pros and Cons of Adenovirus-Based SARS-CoV-2 Vaccines. Mol Ther, 28(11), 2303-2304. doi:10,11016/j.ymthe.2020.10.002

Lana J., Ge, J., Yu, J., Shan, S., Zhou, H., Fan, S., Wang. X. (2020), Structure of the SARS-CoV-2 spike receptor-binding domain bound to the ACE2 receptor. Nature, 581(7807), 215-220. doi:10.1038/41586-020-2180-5

Lee W. S. Wheatley A. K., Kent S. J. & DeKosky (2020). Antibody-dependem enhancement and SARS-CoV-2 vaccines and therapies, Nature Microbiology. VOL 5 1185-1191

Li, W. (2020). Delving deep into the structural aspects of a thrill cleavage site inserted into the spike protein of SARS-CoV-2: A structural biophysical perspective. Biophys Chem, 264, 106420. doi:10:1016/j.bpc.2020.106420

Li, Z., Yi, Y,. Luo, X., Xiong, N., Liu. Y., Li, S., Ye, F., (2020). Development and clinical application of a rapid IgM-IgG combined antibody test :for SARS-CoV-2 infection diagnosis. J Med Virol, 92(9), 1518-1524, doi:10.002/jmv.25727

Long Q-x, Deng H-j, Chen 3, Hu J, Lin B-z, Liao P, et al, Antibody responses to SARS-CoV-2 COV D-19 patients: the perspective application of serological tests in clinical practice. medRxiv. 2020:2020.03.18.20038018.

Maliase, E. (2021). Covid-19: Novavax vaccine efficacy is 86% against UK variant and 60% against South African variant. BMJ 372, n296. doi:10.1136/bmj.n296

Maier, H. E. Kuan, G., Saborio, S., Bustos F. A., Plazaola, M., Barilla, C., Gordon, A. (2021), Clinical spectrum of SARS-CoV-2 infection and protection from symptomatic re-infection, Clin infect Dis. doi:1093/cid/clab717

Malik, Y. A. (2020). Properties of Coronavirus and SARS-CoV-2. Malays J Pathol, 42(1), 3-11.

Menni, C., Maser: K. May, A., Polidori, L., Capdevila, J., Louca, P., Spector, T. D. (2021). Vaccine side-effects and SARS-CoV-2 infection after vaccination in users of the COVID Symptom Study app in the UK: a prospective observational study. Lancet Infect Dis, 21(7), 939-949. doi:10.1016/S1473-3099(21)00224-3

Meyers, L. M, Gutierrez, A. H., Boyle, C. M., Terry, F., McGonnigal, B, G., Salazar, A., Moise. L. (2021). Highly conserved, non-human-like, and cross-reactive SARS-CoV-2 T cell epitopes for COVID-19 vaccine design and validation. NPJ Vaccines, 6(1), 71. doi:10.1038/s41541-071-00131-6

Nanishi, E., Dowling, D. J., & Levy. O. (2020). Toward precision adjuvants: optimizing science and safety, Curr Opin Pediatr, 32(1), 125-138. doi:10.1097/MOP.000000000000868

NIH Covid treatment guidelines (2021) Clinical Spectrum of SARS-CoV-2 infection: hypertext transfer protocol secure://www.covid19treatmentguidelines.nih.gov/overview/clinical-spectrum/.

Okla N. M. A., Muller M. A., Li W. Wang C. GeurtsvariKessel C H, Corman V M, SARS-CoV-2 specific antibody responses in COVID-19 patients, medRxiv. doi: 2020:2020,03,18.20038059.

Oran, D. P., & Topol, E. J. (2021). The Proportion of SARS-COV-2 Infections That Are Asymptomatic: A Systematic Review. Ann Intern Med, 174(5), 655-662. doi:10.7326/M20-6976

Oran D. P. and Topol E.L. (2020) The Proportion of SARS-CoV-2 infections that are asymptomatic: A Systematic Review. Ann Intern Med, doi:10.7326/M20-6976

Pallesen, J., Wang, N., Corbett, K. S., Wrapp, D., Kirehdoerfcr, R. N., Turner, H, L., McLellan, J. S. (2017). Immunogenicity and structures or a rationally designed prefusion MERS-CoV spike antigen. Proc Natl Acad Sci U S A, 114(35), E7348-E7357. doi:10.1073/pnas.1707304114

Polack, F. P., Thomas, S. J., Kitchin, N., Absalon, J., Gurtman, A., Lockhart, S., Group, C. C. T. (2020). Safety and Efficacy of the BNT162b2 mRNA Covid-19 Vaccine, N Engl J Med, 383(27), 2603-2615, doi:10.1056/NEJMoa2034577

Prompetchara E. Ketloy Palaga T. (2020) Immune responses in COVID-19 and potential vaccines; lessons learned from SARS and MFRS epidemic. Asian Pac J Allergy Immanol, 38 (1):1-9, dot:10.17917SAP-200220-0772.

Ravi, N., Cortade, D. L., Ng, E., & Wang, S. X. (2020). Diagnostics for SARS-CoV-2 detection: A comprehensive review of the FDA-EUA COVID-19 testing landscape. Biosens Bioelectron, 165, 112454, doi: 10.10161/j.bios.2020.112454

Risson, E. (2020), inactivated vaccine for SARS-CoV-2. Nat Rev Immunol, 20(6), 353. doi: 10.1038/s41577-020-)314-1

Roberts, A., Lamirande, E. W., Vogel, L., Baras, B., Goossens, G., Knott, I., Subbarao, K. (2010). Immunogenicity and protective efficacy in mice and hamsters of a beta-propiolactone inactivated whole virus SARS-CoV vaccine. Viral immune, 23(5), 509-519. doi:10.1089vim.2010.0028

Roser, M., Ritchie, H., Ortiz-Ospina, E., and Basell, J. (2020). Coronavirus Pandemic (COVID-19) hypertext transfer protocol secure://ourworldindata.orgicoronavirus.

Ryzhikov et al. Peptides immunogens and vaccine composition against COVID19 with the use of peptides immuncigens. Rusian Patent No 2.743593 (C). 20-2-2021

Saha, S., & Raghava, G. P. (2006) AlgPred: prediction of allergenic proteins and mapping of IgE epitopes. Nucleic Acids Res, 34(Web Server issue), W202-209, doi:10.1093/nar/gk1343

Samavati., L., & Uhal, B. D. (2020). CE2, Mach More Than just a Receptor for SARS-COV-2. Front Cell Infect Microbiol. 10, 317, doi:10.3389/fcimb.2020.00317

Schlake, T., Thess, A., Fotin-Mleczek, M., & Kallen, K. J. (2012). Developing mRNA-vaccine technologies. RNA Biol, 9(11), 1319-1330. doi:10.4161/ma.22269

Sethuraman, N., Jeremiah, S. S., & Ryo, A. (2020). Intetpreting Diagnostic. Tests for SARS-CoV-2. JAMA. 323(22), 2249-2251. doi:10.1001/jama.2020.8259

Shaleme, T., Bekele, F., &. Ayela, T. (2020). Clinical Presentation of Patients Infected with Coronavirus Disease 19: A Systematic Review. Infect Dis (Auckl) 13 , 1178633720952076. doi:10,1177/1178633720952076

Siaramella G. et al. Beta Coronaviriss mRNA vaccine, U.S. Pat. No. 10,702600. (Jul. 7, 2020)

Siddell, S. G., Anderson, R., Cavanatth, D., Fujiwara, K., Klenk, H. D., Macnatighton, M K., Van der Zeijst B. A. (1983). Coronaviridae. Intervirology, 20(4), 181-189. doi.:10.1159/000149390

Singh, A., Thakur, M., Sharma, L. K., & Chandra, K. (2020). Designing a multi-epitope peptide based vaccine against SARS-CoV-2. Sci Rep, 10(1), 16219, (doi:10.1038/5415980073371-y

Song: J., Tan, H., Perry, A. J, Akursu, T., Webb, G. Whisstock, J. C., & Pike, R. N. (2012). PROSPER: an integrated feature-based tool for predicting protease substrate cleavage sites. PLoS One, 7(11), e50300. doi:10.1371/journal.pone/0050300

Sorel, G., Faiyre, B. & Mor and, S. (2020). Explaining among-country variation in COVID-19 case fatality rate. Sci Rep, 10(1), 18909, doi:10.1038/s41598-020-75848-2

Sun, W., He, L., Zhang, H., Tian, X., Bai, Z., Sun, L., Li, (2021). The sell-assembled nartoparticle-based trimeric RBD mRNA vaccine elicits robust and durable protective immunity against SARS-CoV-2 in mice. Signal Transduct Target Ther, 6(1), 340. doi:10.1038/s541392-021-00750-w

Tanriover, M. D Dog,artay, H. L., Akova, M., Guner, H. R., Azap, A Akhan, S., CoronaVac Study, G. (2021), Efficacy and safety of an inactivated whole-virion SARS-CoV-2 vaccine (CoronaVac): interim results of a double-blind, randomised, placebo-controlled, phase 3 trial in Turkey. Lancet, 398(10296), 213-222. doi:10.1016/S0140-6736(21)01429-X

Tatsis, N., & Ertl, H. C. (2004). Adenoviruses as vaccine vectors, Mol Thor, 10(4), 616-629. doi:10.1016.j.ymthe.2004.07.013

Thomas, S. J., Moneira, E. D., Jr., Kitchin, N., Absalon, J., Gurtman, A., Lockhart, S., Group, C. C. T. (2021), Safety and Efficacy of the BNT162b2 mRN A Covid-19 Vaccine through 6 Months. N Engl J Med. doi:10.1056/NEJMoa2110345

Thompson, D., Delorme, C. M., White, R. F., & Honer, W. G. (2021). Elevated clozapine levels and toxic effects after SARS-CoV-2 vaccination. J Psychiaity Neurosci, 46(2), E210-E211. dio:10.1503/jpn 211027.

Tian S., Huajun W., Wu J. (2012) Compatational prediction of fttrin cleavage sites by a hybrid method and understanding mechanism underlying diseases. Scientific Reports, 2, 261

Tian S., Huang Q., Fang Y. Wu J. (2011) FurinDB: a database of 20-residue fur in cleavage site motifs, substrates and their associated drugs. International Journal of Molecular Sciences., 12, 1060-1065.

Tipnis, S. K., Hooper, N. M., Hyde, R., Kaman, E., Christie, G., & Turner, A. J. (2000). A human homolog of angiotensin-converting enzyme, Cloning and functional expression as a captopril-insensitive carboxypeptidase. J Biol Chem, 275(43), 33238-33243. doi:10.1074/jpc.M002615200

Trogen, B., Oshinsky, D., & Caplan, A. (2020). Adverse Consequences of Rushing a SARS-CoV-2 Vaccine: Implications for Public Trust. JAMA, 323(24), 2460-2461, doi:10.1001/jama:2020.8917

Tsaneva-Damyanova (2020) D. SARS-CoV-2: seroepidemiological pattern in northeastern Bulgaria. Biotechnology & Biotechnological Equipment 34(1)441-6,

Vankadari, N. (2020), Structure of Furin Protease Binding to SARS-CoV-2 Spike Glycoprotein and Implications for Poternial Targets and Virulence. J Phys Chem Lett, 11(16), 6655-6663, doi:10.1021/acsjpclett,0c01698

Vashi, Y., Jagrit, V., & Kumar, S. (2020). Understanding the B and T cell epitopes of spike protein of severe acute respiratory syndrome corortavirus-2: A computational way to predict the immunogens. Infect Gene Evol, 84, 104382. doi:10.10161/j.meegid.2020.104382

Verity, R., Okell, L. C., Dorigatti, I., Winskill, P., Whittaker., C., Imai, N., Ferguson, N. M. (2020). Estimates of the severity of corortavirus disease 2019: a model-based analysis. Lancet Infect Dis, 20(6), 669-677, doi: 10.1016/S1473-3099(20)30243-7

Wajnberg, A., Amanat, F., Firpo, A., Altman, D. R., Bailey, M.J., Mansour, M., Cordon-Cardo, C. (2020). Robust neutralizing antibodies to SARS-CoV-2 infection persist for months: Science, 370(6521), 1227-1230. doi:10.1126/science.abd7728

Walls, A. C. Park, Y. J., Tortorici, M. A., Wall, A., McGuire, A. T., & Veesler. D. (2020). Structure, Function, and Anticenicity of the SARS-COV-2 Spike. Glycoprotein. Cell, 881(2), 281-292 e286. doi:10.1016/j.cell.2020.02.058

Weiskopf, D., Schmitz, K. S., Raadsen, M. P., Grifoni, A., Okla, N, M. A., Endernan, H., de Vries, R, D. (2020): Phenotype and kinetics of SARS-CoV-2-specific. T cells in COVID-19 patients with acute respiratory distress syndrome, Sci Immynol, 5(48). doi:10.1126/sciimmurtol.abd2071

WHO (2021) Background document to the WHO Interim recommendations for use of Ad26.COV2.S (COVID-19) vaccine. March 2021

Wilder-Smith. A., Mulholland, K. (2021). Effectiveness of an Inactivated SARS-CoV-2 Vaccine. N Engl. J Med, 385(10), 946-948. doi:10.1056/NEJMe2111165

Wrapp, D., Wang, N., Corbett, K. S. Goldsmith, J. A., Hsieh. C. L., Abiona, O., McLellan, J. S. (2020), Cryo-EM Structure of the 2019-nCoV Spike in the Prefusion Conformation. bioRxiv, doi:10.1101/2020.02.11.944462

Woelfel R, Corman V M, Guggemos W. Settmaier M, Zange S. Mueller M A, at al. Clinical presentation and virological assessment of hospitalized cases of coronavirus disease 2019 in a travel-associated transmission cluster, medRxiv. 2020:2020.03,05.20030502.

Wu, L. P., Wang., N. C., Chang, Y. H., Tian, X. Y., Na, D. Y., Zhang, L, Y., Liana, G. D. (2007). Duration of antibody responses after severe acute respiratory syndrome. Emerg Infect Dis. 13(10), 1562-1564, doi:10.3201/eid1310.070576

Wu, Z., Hu, Y., Na, M., Chen, Z., Yang, W., Jiang, Z., Yin, W. (2021). Safety, tolerability, and immunogenicity of an inactivated. SARS-CoV-2 vaccine (CoronaVac) in healthy adults aged 60 years and older: a randomised, double-blind, placebo-controlled, phitse ½ clinical trial. Lancet Infect Dis. 21(6), 803-812, doi:10.1016/S1473-3099(20)30987-7

Xiang et al. Producing recombinant SARS-CoV-2 Spike protein in a prefusion state. U.S. Pat. No. 11,020,474 B1 (Jun. 1, 2021)

Xiao, A. T., Gao, C., & Zhang, S. (2020). Profile of specific antibodies to SARS-CoV-2: The first report, J Infect, 81(1),147-178. doi:10.1016/j.jing.2020.03.012

Yan, R., Mang, Y., Li, Y., Xia, L., Guo, & Zhou, Q. (2020). Structural basis for the recognition of SARS-CoV-2 by full-length human ACE 2, Science, 367(6485). 1444-1448, doi:10.1126/science.abb2762

Yang, a, Chertov, O., Bykovsckrtia, S. N. (Then, Q., Buffo, M, S., Shogan, i. Oppenheim, J. (1999). Beta-defensins: linking innate and adaptive immunity through dendritic and. T cell CCR6. Science, 286(5439), 525-528. doi:10.1126/science.286.5439.525

Zak, D. E., Andersen-Nissten, E., Peterson, E. R., Sato, A., Hamilton, M. K . Borgerding, J., McElrath M. J. (2012). Merck Ad5/HIV induces broad innate immune activation that predicts CD8(+) T-cell responses but is attenuated by preexisting Ad5 immunity. Proc Natl. Acad Sci U S A, 109(50), E3503-3512. doi:101073/pnas.1208972109

Zeng, H Xu, C., Fan, J., Tang, Y., Deng, Q., Zhang, & Long, X. (2020). Antibodies in Infants Born to Mothers With COVID-19 Pneumonia. JAMA, 323(18), 1848-1849, doi:10.1001/jama2020.4861

Zha, L., Chang, X., Zhao, H., Mohsen. M. O., Hong, L. Zhou, Y., Bachmann, M. F. (2021). Development of a Vaccine against SARS-CoV-2 Based on the Receptor-Binding, Domain Displayed on Virus-Like Particles. Vaccines (Basel), 9(4), doi:10.3390/vaccines9040395

Zhao, J., Yuan. Q., Wang, IL, Liu, W., Liao, X., Su, Y., Zhang, Z. (2020), Antibody Responses to SARS-CoV-2 in Patients With Novel Coronavirus Disease 2019, Clin Infect Dis. 71(16), 2027-2034, doi:10.1093/cid/ciaa344

Zhao. Y., Zhao, Z., Wang. Y., Zhou, Y., Ma, Y. & Zuo. W, (2020. Single-Cell RNA Expression Profiling of ACE2, the Receptor of SARS-CoV-2. Am J Respir Crit Care Med, 202(5), 756-759. doi:10.1164rccm.202001-0179LE

1-76. (canceled)

77. A polypeptide antigen comprising a SARS-CoV-2 S protein S1; S2 cut site or a S1/S′2 cut site, including the P3 peptide (SEQ ID NO: 3), a variant thereof, or a chemically modified form thereof.

78. The polypeptide antigen of claim 77, further comprising a Turin cut site or a Transmembrane Protease Serine 2 (TMPRSS2) cut site.

79. The polypeptide antigen of claim 77 comprising a peptide selected from the group consisting of P3 (SEQ ID NO: 3), P3 L (SEQ ID NO: 43), SJ-FT (SEQ ID NO: 144) and P4/TMP-Pol3 (SEQ ID NO: 116), SEQ ID NOs: 1, 2, 4-39, 47, 51, 55, 59, 64-74, 108, 110-115, 117-120, 148, 152, 156, 160, and 164; or a chemically modified form thereof.

80. The polypeptide antigen of claim 77 comprising a peptide selected from the group consisting of P3 (SEQ ID NO: 3), P3 L (SEQ ID NO: 43), SJ-FT (SEQ ID NO: 144) and P4/TMP-Pol3 (SEQ ID NO: 116) or a variant thereof or a recombinant or an engineered or chemically modified form thereof.

81. The polypeptide antigen of claim 77 that further comprises an N-terminal biotin group.

82. The polypeptide antigen of claim 77 that comprises a peptide selected from the group consisting of SEQ ID NOs: 1-30, 64-74, and 110-120.

83. The polypeptide antigen claim 77, wherein the peptide is selected from the group consisting of SEQ ID NOs: 1-15.

84. The polypeptide antigen of claim 77, wherein the peptide is selected from the group consisting of SEQ ID NOs: 1-10 and 110-120.

85. The polypeptide antigen of claim 77, wherein the peptide is SEQ ID NO: 3.

86. The polypeptide antigen of claim 77, wherein the peptide is selected from the group consisting of SEQ ID NOs: 39, 43, 47, 51, 55, 59, 144, 148, 152, 156, 160, and 164.

87. The polypeptide antigen of claim 77, wherein the peptide is one or more peptides selected from the group consisting of SEQ ID NOs: 1-7, 9-12, 14-15, 18, 31-38, and 108.

88. The polypeptide antigen of claim 77, wherein the peptide is one or more peptides selected from the group consisting of SEQ ID NO: 8 and SEQ ID NO: 13.

89. The polypeptide antigen of claim 77 that is a wild-type peptide that comprises 6 to 20 contiguous amino acid residue of a SARS-CoV-2 antigen.

90. The polypeptide antigen of claim 77 that is a chimeric antigen or peptide or a peptide or antigen conjugate comprising the at least one peptide or antigen of claim 77.

91. The polypeptide antigen of claim 77 that has been chemically modified to comprise an N-terminal biotin group, or at least one other exogenous chemical group or chemical modification.

92. A composition comprising the polypeptide antigen of claim 77, or an engineered or chemically modified form thereof or a chemically modified form thereof, and a carrier, excipient, and/or adjuvant.

93. The composition of claim 92 that comprises two or more of said polypeptide antigens.

94. The composition of claim 92 that is immunogenic and which induces humor al or cellular immunity against SARS-CoV-2 when administered to a subject.

95. A method for preventing or reducing the severity of an infection by SARS-CoV-2 comprising administering the polypeptide antigen of claim 77, or a chemically modified form thereof, to a subject in need thereof.

96. A method for detecting antibodies to SARS-CoV-2 in a biological sample comprising contacting the sample with at least one antigen or a peptide, chimeric antigen or peptide, or peptide conjugate of claim 77, or a chemically modified form thereof

97. A nucleic acid encoding at least one polypeptide antigen of claim 77 or a peptide, a chimeric antigen or a peptide, or an antigen or a polypeptide antigen conjugate or an engineered or chemically modified form thereof

98. A composition comprising the nucleic acid of claim 97 and a pharmaceutically acceptable carrier, excipient and/or adjuvant.

99. A method for preventing or reducing the severity of an infection by SARS-CoV-2 comprising administering to a subject in need thereof a nucleic acid encoding the polypeptide antigen of claim 77.

100. A vaccine against SARS-CoV-2 infection comprising the polypeptide antigen of claim 77 or a variant thereof or a recombinant or an engineered or chemically modified form thereof.


SEQUENCE LISTING (171 sequences)

SEQ ID NO: 1 - protein, 21 aa, synthetic construct
  REGION 1..21: note = Designed peptide P1
GGGGSQTNSPRRARSVASQSI

SEQ ID NO: 2 - protein, 42 aa, synthetic construct
  REGION 1..42: note = Designed peptide P2
GGGGSQTNSPRRARSVASQSIGGGGSQTNSPRRARSVASQSI

SEQ ID NO: 3 - protein, 63 aa, synthetic construct
  REGION 1..63: note = Designed peptide P3
GGGGSQTNSPRRARSVASQSIGGGGSQTNSPRRARSVASQSIGGGGSQTNSPRRARSVASQSI

SEQ ID NO: 4 - protein, 21 aa, synthetic construct
  REGION 1..21: note = Designed peptide P4
GGGGSQCNSPRRARSVASQCI

SEQ ID NO: 5 - protein, 26 aa, synthetic construct
  REGION 1..26: note = Designed peptide P5
GGGGSQCNSPRRARSVASQCIGGGGS

SEQ ID NO: 6 - protein, 15 aa, synthetic construct
  REGION 1..15: note = Designed peptide P19
GGGGSSPRRARSVAS

SEQ ID NO: 7 - protein, 45 aa, synthetic construct
  REGION 1..45: note = Designed peptide P21
GGGGSSPRRARSVASGGGGSSPRRARSVASGGGGSSPRRARSVAS

SEQ ID NO: 8 - protein, 14 aa, synthetic construct
  REGION 1..14: note = Designed peptide P22
GGGGSQTQTNSPRR

SEQ ID NO: 9 - protein, 14 aa, synthetic construct
  REGION 1..14: note = Designed peptide P23
GGGGSQTNSPRRAR

SEQ ID NO: 10 - protein, 14 aa, synthetic construct
  REGION 1..14: note = Designed peptide P24
GGGGSNSPRRARSV

SEQ ID NO: 11 - protein, 16 aa, synthetic construct
  REGION 1..16: note = Designed peptide P1(v)
QTNSPRRARSVASQSI

SEQ ID NO: 12 - protein, 10 aa, synthetic construct
  REGION 1..10: note = Designed peptide P19(v)
SPRRARSVAS

SEQ ID NO: 13 - protein, 9 aa, synthetic construct
  REGION 1..9: note = Designed peptide P22(v)
QTQTNSPRR

SEQ ID NO: 14 - protein, 9 aa, synthetic construct
  REGION 1..9: note = Designed peptide P23(v)
QTNSPRRAR

SEQ ID NO: 15 - protein, 9 aa, synthetic construct
  REGION 1..9: note = Designed peptide P24(v)
NSPRRARSV

SEQ ID NO: 16 - protein, 13 aa, synthetic construct
  REGION 1..13: note = Designed peptide P1-B7
SPRRARSVASQSI

SEQ ID NO: 17 - protein, 8 aa, synthetic construct
  REGION 1..8: note = Designed peptide P19-B8
SPRRARSV

SEQ ID NO: 18 - protein, 9 aa, synthetic construct
  REGION 1..9: note = Designed peptide P19-B7
SPRRARSVA

SEQ ID NO: 19 - protein, 9 aa, synthetic construct
  REGION 1..9: note = Designed peptide PepF25
IAIPTNFTI

SEQ ID NO: 20 - protein, 9 aa, synthetic construct
  REGION 1..9: note = Designed peptide PepF24
NSIAIPTNF

SEQ ID NO: 21 - protein, 9 aa, synthetic construct
  REGION 1..9: note = Designed peptide PepF23
SVAYSNNSI

SEQ ID NO: 22 - protein, 9 aa, synthetic construct
  REGION 1..9: note = Designed peptide PepF22
SIIAYTMSL

SEQ ID NO: 23 - protein, 9 aa, synthetic construct
  REGION 1..9: note = Designed peptide PepF21
SQSIIAYTM

SEQ ID NO: 24 - protein, 9 aa, synthetic construct
  REGION 1..9: note = Designed peptide PepF20
VASQSIIAY

SEQ ID NO: 25 - protein, 10 aa, synthetic construct
  REGION 1..10: note = Designed peptide PepF20-/A26-B15
SVASQSIIAY

SEQ ID NO: 26 - protein, 9 aa, synthetic construct
  REGION 1..9: note = Designed peptide PepF19
RSVASQSII

SEQ ID NO: 27 - protein, 7 aa, synthetic construct
  REGION 1..7: note = Designed peptide PepF10
IPTNFTI

SEQ ID NO: 28 - protein, 9 aa, synthetic construct
  REGION 1..9: note = Designed peptide PepF7
IGAGICASY

SEQ ID NO: 29 - protein, 9 aa, synthetic construct
  REGION 1..9: note = Designed peptide PepF6
CDIPIGAGI

SEQ ID NO: 30 - protein, 9 aa, synthetic construct
  REGION 1..9: note = Designed peptide PepF5
ECDIPIGAG

SEQ ID NO: 31 - protein, 58 aa, synthetic construct
  REGION 1..58: note = Designed peptide P3'
QTNSPRRARSVASQSIGGGGSQTNSPRRARSVASQSIGGGGSQTNSPRRARSVASQSI

SEQ ID NO: 32 - protein, 13 aa, synthetic construct
  REGION 1..13: note = Designed peptide P19-B7'
GGGGSPRRARSVA

SEQ ID NO: 33 - protein, 18 aa, synthetic construct
  REGION 1..18: note = Designed peptide P19-B7"
GGGGSPRRARSVAGGGGS

SEQ ID NO: 34 - protein, 11 aa, synthetic construct
  REGION 1..11: note = Designed peptide P19'
SSPRRARSVAS

SEQ ID NO: 35 - protein, 37 aa, synthetic construct
  REGION 1..37: note = Designed peptide P2(v)
QTNSPRRARSVASQSIGGGGSQTNSPRRARSVASQSI

SEQ ID NO: 36 - protein, 16 aa, synthetic construct
  REGION 1..16: note = Designed peptide P4(v)
QCNSPRRARSVASQCI

SEQ ID NO: 37 - protein, 20 aa, synthetic construct
  REGION 1..20: note = Variant of Peptide AGU-P4
QCNSPRRARSVASQCIGGGS

SEQ ID NO: 38 - protein, 22 aa, synthetic construct
  REGION 1..22: note = Designed peptide P5(v)
SQCNSPRRARSVASQCIGGGGS

SEQ ID NO: 39 - protein, 64 aa, synthetic construct
  REGION 1..64: note = Designed peptide P3
MGGGGSQTNSPRRARSVASQSIGGGGSQTNSPRRARSVASQSIGGGGSQTNSPRRARSVASQSI

SEQ ID NO: 40 - DNA, 192 bp, synthetic construct
  misc_feature 1..192: note = P3 optimized cDNA
  source 1..192: mol_type = other DNA
atgggcggcggcggcagccagaccaacagccccaggagggccaggagcgtggccagccagagcatcggcggcggcggcagccagaccaacagccccaggagggccaggagcgtggccagccagagcatcggcggcggcggcagccagaccaacagccccaggagggccaggagcgtggccagccagagcatc

SEQ ID NO: 41 - DNA, 192 bp, synthetic construct
  misc_feature 1..192: note = P3 E. coli K12 Optimized cDNA
  source 1..192: mol_type = other DNA
atgggcggcggcggcagccagaccaacagcccgcgccgcgcgcgcagcgtggcgagccagagcattggcggcggcggcagccagaccaacagcccgcgccgcgcgcgcagcgtggcgagccagagcattggcggcggcggcagccagaccaacagcccgcgccgcgcgcgcagcgtggcgagccagagcatt

SEQ ID NO: 42 - DNA, 192 bp, synthetic construct
  misc_feature 1..192: note = P3 Yeast (Saccharomyces) Optimized cDNA
  source 1..192: mol_type = other DNA
atgggtggtggtggttctcaaactaattctccaagaagagctagatctgttgcttctcaatctattggtggtggtggttctcaaactaattctccaagaagagctagatctgttgcttctcaatctattggtggtggtggttctcaaactaattctccaagaagagctagatctgttgcttctcaatctatt

SEQ ID NO: 43 - protein, 107 aa, synthetic construct
  REGION 1..107: note = Designed P3-L peptide
MASYQTQTNSPRRARSVASQSIIAYTMSLGAENGGGGSASYQTQTNSPRRARSVASQSIIAYTMSLGAENGGGGSASYQTQTNSPRRARSVASQSIIAYTMSLGAEN

SEQ ID NO: 44 - DNA, 321 bp, synthetic construct
  misc_feature 1..321: note = P3-L Homo sapiens (Mammalian Cells) cDNA optimized sequence
  source 1..321: mol_type = other DNA
atggccagctaccagacccagaccaacagccccaggagggccaggagcgtggccagccagagcatcatcgcctacaccatgagcctgggcgccgagaacggcggcggcggcagcgccagctaccagacccagaccaacagccccaggagggccaggagcgtggccagccagagcatcatcgcctacaccatgagcctgggcgccgagaacggcggcggcggcagcgccagctaccagacccagaccaacagccccaggagggccaggagcgtggccagccagagcatcatcgcctacaccatgagcctgggcgccgagaac

SEQ ID NO: 45 - DNA, 321 bp, synthetic construct
  misc_feature 1..321: note = P3-L E. coli K12 Optimized cDNA
  source 1..321: mol_type = other DNA
atggcgagctatcagacccagaccaacagcccgcgccgcgcgcgcagcgtggcgagccagagcattattgcgtataccatgagcctgggcgcggaaaacggcggcggcggcagcgcgagctatcagacccagaccaacagcccgcgccgcgcgcgcagcgtggcgagccagagcattattgcgtataccatgagcctgggcgcggaaaacggcggcggcggcagcgcgagctatcagacccagaccaacagcccgcgccgcgcgcgcagcgtggcgagccagagcattattgcgtataccatgagcctgggcgcggaaaac

SEQ ID NO: 46 - DNA, 321 bp, synthetic construct
  misc_feature 1..321: note = P3-L Yeast (Saccharomyces) Optimized cDNA
  source 1..321: mol_type = other DNA
atggcttcttatcaaactcaaactaattctccaagaagagctagatctgttgcttctcaatctattattgcttatactatgtctttgggtgctgaaaatggtggtggtggttctgcttcttatcaaactcaaactaattctccaagaagagctagatctgttgcttctcaatctattattgcttatactatgtctttgggtgctgaaaatggtggtggtggttctgcttcttatcaaactcaaactaattctccaagaagagctagatctgttgcttctcaatctattattgcttatactatgtctttgggtgctgaaaat

SEQ ID NO: 47 - protein, 178 aa, synthetic construct
  REGION 1..178: note = P3-L/AD
MASYQTQTNSPRRARSVASQSIIAYTMSLGAENGGGGSASYQTQTNSPRRARSVASQSIIAYTMSLGAENGGGSASYQTQTNSPRRARSVASQSIIAYTMSLGAENGGGGSRTSYLLLFTLCLLLSEMASGGNFLTGLGHRSDHYNCVSSGGQCLYSACPIFTKIQGTCYRGKAKCCK

SEQ ID NO: 48 - DNA, 534 bp, synthetic construct
  misc_feature 1..534: note = P3-L/AD Homo sapiens (Mammalian Cells) cDNA optimized
  source 1..534: mol_type = other DNA
atggccagctaccagacccagaccaacagccccaggagggccaggagcgtggccagccagagcatcatcgcctacaccatgagcctgggcgccgagaacggcggcggcggcagcgccagctaccagacccagaccaacagccccaggagggccaggagcgtggccagccagagcatcatcgcctacaccatgagcctgggcgccgagaacggcggcggcagcgccagctaccagacccagaccaacagccccaggagggccaggagcgtggccagccagagcatcatcgcctacaccatgagcctgggcgccgagaacggcggcggcggcagcaggaccagctacctgctgctgttcaccctgtgcctgctgctgagcgagatggccagcggcggcaacttcctgaccggcctgggccacaggagcgaccactacaactgcgtgagcagcggcggccagtgcctgtacagcgcctgccccatcttcaccaagatccagggcacctgctacaggggcaaggccaagtgctgcaag

SEQ ID NO: 49 - DNA, 534 bp, synthetic construct
  misc_feature 1..534: note = P3-L/AD E. coli K12 Optimized cDNA
  source 1..534: mol_type = other DNA
atggcgagctatcagacccagaccaacagcccgcgccgcgcgcgcagcgtggcgagccagagcattattgcgtataccatgagcctgggcgcggaaaacggcggcggcggcagcgcgagctatcagacccagaccaacagcccgcgccgcgcgcgcagcgtggcgagccagagcattattgcgtataccatgagcctgggcgcggaaaacggcggcggcagcgcgagctatcagacccagaccaacagcccgcgccgcgcgcgcagcgtggcgagccagagcattattgcgtataccatgagcctgggcgcggaaaacggcggcggcggcagccgcaccagctatctgctgctgtttaccctgtgcctgctgctgagcgaaatggcgagcggcggcaactttctgaccggcctgggccatcgcagcgatcattataactgcgtgagcagcggcggccagtgcctgtatagcgcgtgcccgatttttaccaaaattcagggcacctgctatcgcggcaaagcgaaatgctgcaaa

SEQ ID NO: 50 - DNA, 534 bp, synthetic construct
  misc_feature 1..534: note = P3-L/AD Yeast (Saccharomyces) Optimized cDNA
  source 1..534: mol_type = other DNA
atggcttcttatcaaactcaaactaattctccaagaagagctagatctgttgcttctcaatctattattgcttatactatgtctttgggtgctgaaaatggtggtggtggttctgcttcttatcaaactcaaactaattctccaagaagagctagatctgttgcttctcaatctattattgcttatactatgtctttgggtgctgaaaatggtggtggttctgcttcttatcaaactcaaactaattctccaagaagagctagatctgttgcttctcaatctattattgcttatactatgtctttgggtgctgaaaatggtggtggtggttctagaacttcttatttgttgttgtttactttgtgtttgttgttgtctgaaatggcttctggtggtaattttttgactggtttgggtcatagatctgatcattataattgtgtttcttctggtggtcaatgtttgtattctgcttgtccaatttttactaaaattcaaggtacttgttatagaggtaaagctaaatgttgtaaa

SEQ ID NO: 51 - protein, 81 aa, synthetic construct
  REGION 1..81: note = Designed peptide SJ-80
MNVFQTRAGCLIGAEHVNNSYECDIPIGAGICASYQTQTNSPRRARSVASQSIIAYTMSLGAENSVAYSNNSIAIPTNFTI

SEQ ID NO: 52 - DNA, 243 bp, synthetic construct
  misc_feature 1..243: note = SJ-80 Homo sapiens (Mammalian Cells) optimized DNA
  source 1..243: mol_type = other DNA
atgaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatc

SEQ ID NO: 53 - DNA, 243 bp, synthetic construct
  misc_feature 1..243: note = SJ-80 E. coli K12 Optimized cDNA
  source 1..243: mol_type = other DNA
atgaacgtgtttcagacccgcgcgggctgcctgattggcgcggaacatgtgaacaacagctatgaatgcgatattccgattggcgcgggcatttgcgcgagctatcagacccagaccaacagcccgcgccgcgcgcgcagcgtggcgagccagagcattattgcgtataccatgagcctgggcgcggaaaacagcgtggcgtatagcaacaacagcattgcgattccgaccaactttaccatt

SEQ ID NO: 54 - DNA, 243 bp, synthetic construct
  misc_feature 1..243: note = SJ-80 Yeast (Saccharomyces) Optimized cDNA
  source 1..243: mol_type = other DNA
atgaatgtttttcaaactagagctggttgtttgattggtgctgaacatgttaataattcttatgaatgtgatattccaattggtgctggtatttgtgcttcttatcaaactcaaactaattctccaagaagagctagatctgttgcttctcaatctattattgcttatactatgtctttgggtgctgaaaattctgttgcttattctaataattctattgctattccaactaattttactatt

SEQ ID NO: 55 - protein, 101 aa, synthetic construct
  REGION 1..101: note = Designed peptide SJ-100
MPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTI

SEQ ID NO: 56 - DNA, 303 bp, synthetic construct
  misc_feature 1..303: note = SJ-100 Homo sapiens (Mammalian Cells) cDNA optimized
  source 1..303: mol_type = other DNA
atgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatc

SEQ ID NO: 57 - DNA, 303 bp, synthetic construct
  misc_feature 1..303: note = SJ-100 E. coli K12 Optimized cDNA
  source 1..303: mol_type = other DNA
atgccggtggcgattcatgcggatcagctgaccccgacctggcgcgtgtatagcaccggcagcaacgtgtttcagacccgcgcgggctgcctgattggcgcggaacatgtgaacaacagctatgaatgcgatattccgattggcgcgggcatttgcgcgagctatcagacccagaccaacagcccgcgccgcgcgcgcagcgtggcgagccagagcattattgcgtataccatgagcctgggcgcggaaaacagcgtggcgtatagcaacaacagcattgcgattccgaccaactttaccatt

SEQ ID NO: 58 - DNA, 303 bp, synthetic construct
  misc_feature 1..303: note = SJ-100 Yeast (Saccharomyces) Optimized cDNA
  source 1..303: mol_type = other DNA
atgccagttgctattcatgctgatcaattgactccaacttggagagtttattctactggttctaatgtttttcaaactagagctggttgtttgattggtgctgaacatgttaataattcttatgaatgtgatattccaattggtgctggtatttgtgcttcttatcaaactcaaactaattctccaagaagagctagatctgttgcttctcaatctattattgcttatactatgtctttgggtgctgaaaattctgttgcttattctaataattctattgctattccaactaattttactatt

SEQ ID NO: 59 - protein, 121 aa, synthetic construct
  REGION 1..121: note = Designed peptide SJ-120
MGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTI

SEQ ID NO: 60 - DNA, 363 bp, synthetic construct
  misc_feature 1..363: note = SJ-120 Homo sapiens (Mammalian Cells) optimized cDNA
  source 1..363: mol_type = other DNA
atgggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatc

SEQ ID NO: 61 - DNA, 363 bp, synthetic construct
  misc_feature 1..363: note = SJ-120 E. coli K12 Optimized cDNA
  source 1..363: mol_type = other DNA
atgggcaccaacaccagcaaccaggtggcggtgctgtatcaggatgtgaactgcaccgaagtgccggtggcgattcatgcggatcagctgaccccgacctggcgcgtgtatagcaccggcagcaacgtgtttcagacccgcgcgggctgcctgattggcgcggaacatgtgaacaacagctatgaatgcgatattccgattggcgcgggcatttgcgcgagctatcagacccagaccaacagcccgcgccgcgcgcgcagcgtggcgagccagagcattattgcgtataccatgagcctgggcgcggaaaacagcgtggcgtatagcaacaacagcattgcgattccgaccaactttaccatt

SEQ ID NO: 62 - DNA, 363 bp, synthetic construct
  misc_feature 1..363: note = SJ-120 Yeast (Saccharomyces) Optimized cDNA
  source 1..363: mol_type = other DNA
atgggtactaatacttctaatcaagttgctgttttgtatcaagatgttaattgtactgaagttccagttgctattcatgctgatcaattgactccaacttggagagtttattctactggttctaatgtttttcaaactagagctggttgtttgattggtgctgaacatgttaataattcttatgaatgtgatattccaattggtgctggtatttgtgcttcttatcaaactcaaactaattctccaagaagagctagatctgttgcttctcaatctattattgcttatactatgtctttgggtgctgaaaattctgttgcttattctaataattctattgctattccaactaattttactatt

SEQ ID NO: 63 - protein, 60 aa, synthetic construct
  REGION 1..60: note = S1/S2 designed peptide
ECDIPIGAGICASYQTQTNSPRRARSVASQSIIAYTMSLGAENSVAYSNNSIAIPTNFTI

SEQ ID NO: 64 - protein, 9 aa, synthetic construct
  REGION 1..9: note = PepF25 designed peptide
IAIPTNFTI

SEQ ID NO: 65 - protein, 9 aa, synthetic construct
  REGION 1..9: note = PepF24 designed peptide
NSIAIPTNF

SEQ ID NO: 66 - protein, 9 aa, synthetic construct
  REGION 1..9: note = PepF23 designed peptide
SVAYSNNSI

SEQ ID NO: 67 - protein, 9 aa, synthetic construct
  REGION 1..9: note = PepF22 designed peptide
SIIAYTMSL

SEQ ID NO: 68 - protein, 9 aa, synthetic construct
  REGION 1..9: note = PepF21 designed peptide
SQSIIAYTM

SEQ ID NO: 69 - protein, 9 aa, synthetic construct
  REGION 1..9: note = PepF20
VASQSIIAY

SEQ ID NO: 70 - protein, 9 aa, synthetic construct
  REGION 1..9: note = PepF19 desigend peptide
RSVASQSII

SEQ ID NO: 71 - protein, 7 aa, synthetic construct
  REGION 1..7: note = PepF10 designed peptide
IPTNFTI

SEQ ID NO: 72 - protein, 9 aa, synthetic construct
  REGION 1..9: note = PepF7 designed peptide
IGAGICASY

SEQ ID NO: 73 - protein, 9 aa, synthetic construct
  REGION 1..9: note = PepF6 designed peptide
CDIPIGAGI

SEQ ID NO: 74 - protein, 9 aa, synthetic construct
  REGION 1..9: note = PepF5 designed peptide
ECDIPIGAG

SEQ ID NO: 75 - protein, 11 aa, synthetic construct
  REGION 1..11: note = Designed peptide: PepS1/S2 MHC1
ASYQTQTNSPR

SEQ ID NO: 76 - protein, 9 aa, synthetic construct
  REGION 1..9: note = Designed peptide: PepS1/S2 MHC2
ASYQTQTNR

SEQ ID NO: 77 - protein, 12 aa, synthetic construct
  REGION 1..12: note = Designed peptide: PepS1/S2 MHC3
QTNSPRRARSVA

SEQ ID NO: 78 - protein, 9 aa, synthetic construct
  REGION 1..9: note = Designed peptide: PepS1/S2 MHC4
QPRRARSVA

SEQ ID NO: 79 - protein, 11 aa, synthetic construct
  REGION 1..11: note = Designed peptide: PepS1/S2 MHC5
TNSPRRARSVA

SEQ ID NO: 80 - protein, 9 aa, synthetic construct
  REGION 1..9: note = Designed peptide: PepS1/S2 MHC6
TPRRARSVA

SEQ ID NO: 81 - protein, 10 aa, synthetic construct
  REGION 1..10: note = Designed peptide: PepS1/S2 MHC7
NSPRRARSVA

SEQ ID NO: 82 - protein, 9 aa, synthetic construct
  REGION 1..9: note = Designed peptide: PepS1/S2 MHC8
NPRRARSVA

SEQ ID NO: 83 - protein, 9 aa, synthetic construct
  REGION 1..9: note = Designed peptide: PepS1/S2 MHC9
SPRRASVAS

SEQ ID NO: 84 - protein, 11 aa, synthetic construct
  REGION 1..11: note = Designed peptide: PepS1/S2 MHC10
SPRRARSVASQ

SEQ ID NO: 85 - protein, 9 aa, synthetic construct
  REGION 1..9: note = Designed peptide: PepS1/S2 MHC11
SPRRARSSQ

SEQ ID NO: 86 - protein, 14 aa, synthetic construct
  REGION 1..14: note = Designed peptide: PepS1/S2 MHC12
SPRRARSVASQSII

SEQ ID NO: 87 - protein, 9 aa, synthetic construct
  REGION 1..9: note = Designed peptide: PepS1/S2 MHC13
SPRRARSVI

SEQ ID NO: 88 - protein, 9 aa, synthetic construct
  REGION 1..9: note = Designed peptide: PepS1/S2 MHC14
RRARSVASQ

SEQ ID NO: 89 - protein, 11 aa, synthetic construct
  REGION 1..11: note = Designed peptide: PepS1/S2 MHC15
RRARSVASQSI

SEQ ID NO: 90 - protein, 9 aa, synthetic construct
  REGION 1..9: note = Designed peptide: PepS1/S2 MHC16
RRARSVQSI

SEQ ID NO: 91 - protein, 14 aa, synthetic construct
  REGION 1..14: note = Designed peptide: PepS1/S2 MHC17
RRARSVASQSIIAY

SEQ ID NO: 92 - protein, 9 aa, synthetic construct
  REGION 1..9: note = Designed peptide: PepS1/S2 MHC18
RRAQSIIAY

SEQ ID NO: 93 - protein, 10 aa, synthetic construct
  REGION 1..10: note = Designed peptide: PepS1/S2 MHC19
RARSVASQSI

SEQ ID NO: 94 - protein, 9 aa, synthetic construct
  REGION 1..9: note = Designed peptide: PepS1/S2 MHC20
RARSASQSI

SEQ ID NO: 95 - protein, 9 aa, synthetic construct
  REGION 1..9: note = Designed peptide: PepS1/S2 MHC21
ARSVASQSI

SEQ ID NO: 96 - protein, 12 aa, synthetic construct
  REGION 1..12: note = Designed peptide: PepS1/S2 MHC22
ARSVASQSIIAY

SEQ ID NO: 97 - protein, 9 aa, synthetic construct
  REGION 1..9: note = Designed peptide: PepS1/S2 MHC23
ARSQSIIAY

SEQ ID NO: 98 - protein, 11 aa, synthetic construct
  REGION 1..11: note = Designed peptide: PepS1/S2 MHC24
RSVASQSIIAY

SEQ ID NO: 99 - protein, 9 aa, synthetic construct
  REGION 1..9: note = Designed peptide: PepS1/S2 MHC25
RSSQSIIAY

SEQ ID NO: 100 - protein, 9 aa, synthetic construct
  REGION 1..9: note = Designed peptide: PepS1/S2 MHC26
RASQSIIAY

SEQ ID NO: 101 - protein, 9 aa, synthetic construct
  REGION 1..9: note = Designed peptide: PepS1/S2 MHC27
SVASQSIAY

SEQ ID NO: 102 - protein, 9 aa, synthetic construct
  REGION 1..9: note = Designed peptide: PepS1/S2 MHC28
SVASSIIAY

SEQ ID NO: 103 - protein, 9 aa, synthetic construct
  REGION 1..9: note = Designed peptide: PepS1/S2 MHC29
SPRRASQSI

SEQ ID NO: 104 - protein, 9 aa, synthetic construct
  REGION 1..9: note = Designed peptide: PepS1/S2 MHC30
SVASQIIAY

SEQ ID NO: 105 - protein, 9 aa, synthetic construct
  REGION 1..9: note = Designed peptide: PepS1/S2 MHC31
SVAQSIIAY

SEQ ID NO: 106 - protein, 4 aa, synthetic construct
  REGION 1..4: note = Furin cleavage site 1 (FCS1)
RRAR

SEQ ID NO: 107 - protein, 5 aa, synthetic construct
  REGION 1..5: note = FCS2 motif
PRRAR

SEQ ID NO: 108 - protein, 26 aa, synthetic construct
  REGION 1..26: note = Designed Peptide 3 variant
GGGGSQTNSPRRARSVASQSIGGGGS

SEQ ID NO: 109 - protein, 80 aa, synthetic construct
  REGION 1..80: note = Designed peptide:S1/S'2 Junction
TPPIKDFGGFNFSQILPDPSKPSKRSFIEDLLFNKVTLADAGFIKQYGDCLGDIAARDLICAQKFNGLTVLPPLLTDEMI

SEQ ID NO: 110 - protein, 9 aa, synthetic construct
  REGION 1..9: note = Designed peptide P1/TMP
ICAQKFNGL

SEQ ID NO: 111 - protein, 9 aa, synthetic construct
  REGION 1..9: note = Designed peptide P2/DMP
RDLICAQKF

SEQ ID NO: 112 - protein, 9 aa, synthetic construct
  REGION 1..9: note = Designed peptide P3/TMP
IEDLLFNKV

SEQ ID NO: 113 - protein, 9 aa, synthetic construct
  REGION 1..9: note = Designed peptide P4/TMP
PDPSKPSKR

SEQ ID NO: 114 - protein, 10 aa, synthetic construct
  REGION 1..10: note = motif
KPSKRSFIED

SEQ ID NO: 115 - protein, 14 aa, synthetic construct
  REGION 1..14: note = Designed peptide P4/TMP-bn
GGGGSPDPSKPSKR

SEQ ID NO: 116 - protein, 42 aa, synthetic construct
  REGION 1..42: note = Designed peptide P4/TMP-Pol3
GGGGSPDPSKPSKRGGGGSPDPSKPSKRGGGGSPDPSKPSKR

SEQ ID NO: 117 - protein, 42 aa, synthetic construct; identical to SEQ ID NO: 116
  REGION 1..42: note = Designed peptide P4/TMP-Pol3-bn

SEQ ID NO: 118 - protein, 14 aa, synthetic construct
  REGION 1..14: note = Designed peptide P3/TMP-bn
GGGGSIEDLLFNKV

SEQ ID NO: 119 - protein, 13 aa, synthetic construct
  REGION 1..13: note = Designed peptide P2/TMP-bn
GGGSRDLICAQKF

SEQ ID NO: 120 - protein, 14 aa, synthetic construct
  REGION 1..14: note = Designed peptide P1/TMP-bn
GGGGSICAQKFNGL

SEQ ID NO: 121 - protein, 9 aa, synthetic construct
  REGION 1..9: note = Designed peptide: PepS1/S2 MHC32
LLFNKVTLA

SEQ ID NO: 122 - protein, 9 aa, synthetic construct
  REGION 1..9: note = Designed peptide: PepS1/S2 MHC33
LTVLPPLLT

SEQ ID NO: 123 - protein, 9 aa, synthetic construct
  REGION 1..9: note = Designed peptide: PepS1/S2 MHC34
DLLFNKVTL

SEQ ID NO: 124 - protein, 9 aa, synthetic construct
  REGION 1..9: note = Designed peptide: PepS1/S2 MHC35
  REGION 1..2: note = MISC_FEATURE - Xaa is any amino acid
XXDLLFNKV

SEQ ID NO: 125 - protein, 9 aa, synthetic construct
  REGION 1..9: note = Designed peptide: PepS1/S2 MHC36
  SITE 1: note = MISC_FEATURE - Xaa is any amino acid
XLLFNKVTL

SEQ ID NO: 126 - protein, 9 aa, synthetic construct
  REGION 1..9: note = Designed peptide: PepS1/S2 MHC37
VTLADAGFI

SEQ ID NO: 127 - protein, 9 aa, synthetic construct
  REGION 1..9: note = Designed peptide: PepS1/S2 MHC38
  SITE 9: note = MISC_FEATURE - Xaa is any amino acid
TLADAGFIX

SEQ ID NO: 128 - protein, 9 aa, synthetic construct
  REGION 1..9: note = Designed peptide: PepS1/S2 MHC39
DCLGDIAAR

SEQ ID NO: 129 - protein, 9 aa, synthetic construct
  REGION 1..9: note = Designed peptide: PepS1/S2 MHC40
  SITE 9: note = MISC_FEATURE - Xaa is any amino acid
CLGDIAARX

SEQ ID NO: 130 - protein, 9 aa, synthetic construct
  REGION 1..9: note = Designed peptide: PepS1/S2 MHC41
CLGDIAARD

SEQ ID NO: 131 - protein, 9 aa, synthetic construct
  REGION 1..9: note = Designed peptide: PepS1/S2 MHC42
IKDFGGFNF

SEQ ID NO: 132 - protein, 9 aa, synthetic construct
  REGION 1..9: note = Designed peptide: PepS1/S2 MHC43
FNFSQILPD

SEQ ID NO: 133 - protein, 9 aa, synthetic construct
  REGION 1..9: note = Designed peptide: PepS1/S2 MHC44
FSQILPDPS

SEQ ID NO: 134 - protein, 9 aa, synthetic construct
  REGION 1..9: note = Designed peptide: PepS1/S2 MHC45
KFNGLTVLP

SEQ ID NO: 135 - protein, 9 aa, synthetic construct
  REGION 1..9: note = Designed peptide: PepS1/S2 MHC46
  SITE 9: note = MISC_FEATURE - Xaa is any amino acid
FNGLTVLPX

SEQ ID NO: 136 - protein, 9 aa, synthetic construct
  REGION 1..9: note = Designed peptide: PepS1/S2 MHC47
  SITE 9: note = MISC_FEATURE - Xaa is any amino acid
LFNKVTLAX

SEQ ID NO: 137 - protein, 9 aa, synthetic construct
  REGION 1..9: note = Designed peptide: PepS1/S2 MHC48
FGGFNFSQI

SEQ ID NO: 138 - protein, 9 aa, synthetic construct
  REGION 1..9: note = Designed peptide: PepS1/S2 MHC49
FNGLTVLPP

SEQ ID NO: 139 - protein, 9 aa, synthetic construct
  REGION 1..9: note = Designed peptide: PepS1/S2 MHC50
FIKQYGDCL

SEQ ID NO: 140 - protein, 9 aa, synthetic construct
  REGION 1..9: note = Designed peptide: PepS1/S2 MHC51
KRSFIEDLL

SEQ ID NO: 141 - protein, 9 aa, synthetic construct
  REGION 1..9: note = Designed peptide: PepS1/S2 MHC52
GLTVLPPLL

SEQ ID NO: 142 - protein, 9 aa, synthetic construct
  REGION 1..9: note = Designed peptide: PepS1/S2 MHC53
PPLLTDEMI

SEQ ID NO: 143 - protein, 9 aa, synthetic construct
  REGION 1..9: note = Designed peptide: PepS1/S2 MHC54
  SITE 9: note = MISC_FEATURE - Xaa is any amino acid
PLLTDEMIX

SEQ ID NO: 144 - protein, 230 aa, synthetic construct
  REGION 1..230: note = SJ-FT peptide
ECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGA

SEQ ID NO: 145 - DNA, 690 bp, synthetic construct
  misc_feature 1..690: note = SJ-FT Homo sapiens (Mammalian Cells) cDNA optimized
  source 1..690: mol_type = other DNA
gagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgcc

SEQ ID NO: 146 - DNA, 690 bp, synthetic construct
  misc_feature 1..690: note = SJ-FT E. coli K12 Optimized cDNA
  source 1..690: mol_type = other DNA
gaatgcgatattccgattggcgcgggcatttgcgcgagctatcagacccagaccaacagcccgcgccgcgcgcgcagcgtggcgagccagagcattattgcgtataccatgagcctgggcgcggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcg

SEQ ID NO: 147 - DNA, 690 bp, synthetic construct
  misc_feature 1..690: note = SJ-FT Yeast (Saccharomyces) Optimized cDNA
  source 1..690: mol_type = other DNA
gaatgtgatattccaattggtgctggtatttgtgcttcttatcaaactcaaactaattctccaagaagagctagatctgttgcttctcaatctattattgcttatactatgtctttgggtgctgaaaattctgttgcttattctaataattctattgctattccaactaattttactatttctgttactactgaaattttgccagtttctatgactaaaacttctgttgattgtactatgtatatttgtggtgattctactgaatgttctaatttgttgttgcaatatggttctttttgtactcaattgaatagagctttgactggtattgctgttgaacaagataaaaatactcaagaagtttttgctcaagttaaacaaatttataaaactccaccaattaaagattttggtggttttaatttttctcaaattttgccagatccatctaaaccatctaaaagatcttttattgaagatttgttgtttaataaagttactttggctgatgctggttttattaaacaatatggtgattgtttgggtgatattgctgctagagatttgatttgtgctcaaaaatttaatggtttgactgttttgccaccattgttgactgatgaaatgattgctcaatatacttctgctttgttggctggtactattacttctggttggacttttggtgct

SEQ ID NO: 148 - protein, 140 aa, synthetic construct
  REGION 1..140: note = SJ-S2'/FP1 designed peptide
TQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAM

SEQ ID NO: 149 - DNA, 420 bp, synthetic construct
  misc_feature 1..420: note = SJ-S2'/FP1 Homo sapiens (Mammalian Cells) optimized cDNA
  source 1..420: mol_type = other DNA
acccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatg

SEQ ID NO: 150 - DNA, 420 bp, synthetic construct
  misc_feature 1..420: note = SJ-S2'/FP1 E. coli K12 Optimized cDNA
  source 1..420: mol_type = other DNA
acccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcgcggcgctgcagattccgtttgcgatg

SEQ ID NO: 151 - DNA, 420 bp, synthetic construct
  misc_feature 1..420: note = SJ-S2'/FP1 Yeast (Saccharomyces) Optimized cDNA
  source 1..420: mol_type = other DNA
actcaattgaatagagctttgactggtattgctgttgaacaagataaaaatactcaagaagtttttgctcaagttaaacaaatttataaaactccaccaattaaagattttggtggttttaatttttctcaaattttgccagatccatctaaaccatctaaaagatcttttattgaagatttgttgtttaataaagttactttggctgatgctggttttattaaacaatatggtgattgtttgggtgatattgctgctagagatttgatttgtgctcaaaaatttaatggtttgactgttttgccaccattgttgactgatgaaatgattgctcaatatacttctgctttgttggctggtactattacttctggttggacttttggtgctggtgctgctttgcaaattccatttgctatg

SEQ ID NO: 152 - protein, 220 aa, synthetic construct
  REGION 1..220: note = SJ-S2'/FP1-2 designed peptide
SIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIG

SEQ ID NO: 153 - DNA, 660 bp, synthetic construct
  misc_feature 1..660: note = SJ-S2'/FP1-2 Homo sapiens (Mammalian Cells) optimized cDNA
  source 1..660: mol_type = other DNA
agcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggc

SEQ ID NO: 154 - DNA, 660 bp, synthetic construct
  misc_feature 1..660: note = SJ-S2'/FP1-2 E. coli K12 Optimized cDNA
  source 1..660: mol_type = other DNA
agcattattgcgtataccatgagcctgggcgcggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcgcggcgctgcagattccgtttgcgatgcagatggcgtatcgctttaacggcattggc

SEQ ID NO: 155 - DNA, 660 bp, synthetic construct
  misc_feature 1..660: note = SJ-S2'/FP1-2 Yeast (Saccharomyces) Optimized cDNA
  source 1..660: mol_type = other DNA
tctattattgcttatactatgtctttgggtgctgaaaattctgttgcttattctaataattctattgctattccaactaattttactatttctgttactactgaaattttgccagtttctatgactaaaacttctgttgattgtactatgtatatttgtggtgattctactgaatgttctaatttgttgttgcaatatggttctttttgtactcaattgaatagagctttgactggtattgctgttgaacaagataaaaatactcaagaagtttttgctcaagttaaacaaatttataaaactccaccaattaaagattttggtggttttaatttttctcaaattttgccagatccatctaaaccatctaaaagatcttttattgaagatttgttgtttaataaagttactttggctgatgctggttttattaaacaatatggtgattgtttgggtgatattgctgctagagatttgatttgtgctcaaaaatttaatggtttgactgttttgccaccattgttgactgatgaaatgattgctcaatatacttctgctttgttggctggtactattacttctggttggacttttggtgctggtgctgctttgcaaattccatttgctatgcaaatggcttatagatttaatggtattggt

SEQ ID NO: 156 - protein, 282 aa, synthetic construct
  REGION 1..282: note = SJ-FT/AD designed peptide
ECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGGGGSGNFLTGLGHRSDHYNCVSSGGQCLYSACPIFTKIQGTCYRGKAKCCK

SEQ ID NO: 157 - DNA, 846 bp, synthetic construct
  misc_feature 1..846: note = SJ-FT/AD Homo sapiens (Mammalian Cells) optimized cDNA sequences
  source 1..846: mol_type = other DNA
gagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcggcggcggcagcggcaacttcctgaccggcctgggccacaggagcgaccactacaactgcgtgagcagcggcggccagtgcctgtacagcgcctgccccatcttcaccaagatccagggcacctgctacaggggcaaggccaagtgctgcaag

SEQ ID NO: 158 - DNA, 846 bp, synthetic construct
  misc_feature 1..846: note = SJ-FT/AD E. coli K12 Optimized cDNA
  source 1..846: mol_type = other DNA
gaatgcgatattccgattggcgcgggcatttgcgcgagctatcagacccagaccaacagcccgcgccgcgcgcgcagcgtggcgagccagagcattattgcgtataccatgagcctgggcgcggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcggcggcggcagcggcaactttctgaccggcctgggccatcgcagcgatcattataactgcgtgagcagcggcggccagtgcctgtatagcgcgtgcccgatttttaccaaaattcagggcacctgctatcgcggcaaagcgaaatgctgcaaa

SEQ ID NO: 159 - DNA, 846 bp, synthetic construct
  misc_feature 1..846: note = SJ-FT/AD Yeast (Saccharomyces) Optimized cDNA
  source 1..846: mol_type = other DNA
gaatgtgatattccaattggtgctggtatttgtgcttcttatcaaactcaaactaattctccaagaagagctagatctgttgcttctcaatctattattgcttatactatgtctttgggtgctgaaaattctgttgcttattctaataattctattgctattccaactaattttactatttctgttactactgaaattttgccagtttctatgactaaaacttctgttgattgtactatgtatatttgtggtgattctactgaatgttctaatttgttgttgcaatatggttctttttgtactcaattgaatagagctttgactggtattgctgttgaacaagataaaaatactcaagaagtttttgctcaagttaaacaaatttataaaactccaccaattaaagattttggtggttttaatttttctcaaattttgccagatccatctaaaccatctaaaagatcttttattgaagatttgttgtttaataaagttactttggctgatgctggttttattaaacaatatggtgattgtttgggtgatattgctgctagagatttgatttgtgctcaaaaatttaatggtttgactgttttgccaccattgttgactgatgaaatgattgctcaatatacttctgctttgttggctggtactattacttctggttggacttttggtgctggtggtggtggttctggtaattttttgactggtttgggtcatagatctgatcattataattgtgtttcttctggtggtcaatgtttgtattctgcttgtccaatttttactaaaattcaaggtacttgttatagaggtaaagctaaatgttgtaaa

SEQ ID NO: 160 - protein, 192 aa, synthetic construct
  REGION 1..192: note = SJ-S2'/FP1/AD designed peptide
TQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMGGGGSGNFLTGLGHRSDHYNCVSSGGQCLYSACPIFTKIQGTCYRGKAKCCK

SEQ ID NO: 161 - DNA, 576 bp, synthetic construct
  misc_feature 1..576: note = SJ-S2'/FP1/AD Homo sapiens (Mammalian Cells) optimized cDNA
  source 1..576: mol_type = other DNA
acccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgggcggcggcggcagcggcaacttcctgaccggcctgggccacaggagcgaccactacaactgcgtgagcagcggcggccagtgcctgtacagcgcctgccccatcttcaccaagatccagggcacctgctacaggggcaaggccaagtgctgcaag

SEQ ID NO: 162 - DNA, 576 bp, synthetic construct
  misc_feature 1..576: note = SJ-S2'/FP1/AD E. coli K12 Optimized cDNA
  source 1..576: mol_type = other DNA
acccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcgcggcgctgcagattccgtttgcgatgggcggcggcggcagcggcaactttctgaccggcctgggccatcgcagcgatcattataactgcgtgagcagcggcggccagtgcctgtatagcgcgtgcccgatttttaccaaaattcagggcacctgctatcgcggcaaagcgaaatgctgcaaa

SEQ ID NO: 163 - DNA, 576 bp, synthetic construct
  misc_feature 1..576: note = SJ-S2'/FP1/AD Yeast (Saccharomyces) Optimized cDNA
  source 1..576: mol_type = other DNA
actcaattgaatagagctttgactggtattgctgttgaacaagataaaaatactcaagaagtttttgctcaagttaaacaaatttataaaactccaccaattaaagattttggtggttttaatttttctcaaattttgccagatccatctaaaccatctaaaagatcttttattgaagatttgttgtttaataaagttactttggctgatgctggttttattaaacaatatggtgattgtttgggtgatattgctgctagagatttgatttgtgctcaaaaatttaatggtttgactgttttgccaccattgttgactgatgaaatgattgctcaatatacttctgctttgttggctggtactattacttctggttggacttttggtgctggtgctgctttgcaaattccatttgctatgggtggtggtggttctggtaattttttgactggtttgggtcatagatctgatcattataattgtgtttcttctggtggtcaatgtttgtattctgcttgtccaatttttactaaaattcaaggtacttgttatagaggtaaagctaaatgttgtaaa

SEQ ID NO: 164 - protein, 272 aa, synthetic construct
  REGION 1..272: note = SJ-S2'/FP1-2/AD designed peptide
SIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGGGGGSGNFLTGLGHRSDHYNCVSSGGQCLYSACPIFTKIQGTCYRGKAKCCK

SEQ ID NO: 165 - DNA, 816 bp, synthetic construct
  misc_feature 1..816: note = SJ-S2'/FP1-2/AD Homo sapiens (Mammalian Cells) optimized cDNA
  source 1..816: mol_type = other DNA
agcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcggcggcggcggcagcggcaacttcctgaccggcctgggccacaggagcgaccactacaactgcgtgagcagcggcggccagtgcctgtacagcgcctgccccatcttcaccaagatccagggcacctgctacaggggcaaggccaagtgctgcaag

SEQ ID NO: 166 - DNA, 816 bp, synthetic construct
  misc_feature 1..816: note = SJ-S2'/FP1-2/AD E. coli K12 Optimized cDNA
  source 1..816: mol_type = other DNA
agcattattgcgtataccatgagcctgggcgcggaaaacagcgtggcgtatagcaacaacagcattgcgattccgaccaactttaccattagcgtgaccaccgaaattctgccggtgagcatgaccaaaaccagcgtggattgcaccatgtatatttgcggcgatagcaccgaatgcagcaacctgctgctgcagtatggcagcttttgcacccagctgaaccgcgcgctgaccggcattgcggtggaacaggataaaaacacccaggaagtgtttgcgcaggtgaaacagatttataaaaccccgccgattaaagattttggcggctttaactttagccagattctgccggatccgagcaaaccgagcaaacgcagctttattgaagatctgctgtttaacaaagtgaccctggcggatgcgggctttattaaacagtatggcgattgcctgggcgatattgcggcgcgcgatctgatttgcgcgcagaaatttaacggcctgaccgtgctgccgccgctgctgaccgatgaaatgattgcgcagtataccagcgcgctgctggcgggcaccattaccagcggctggacctttggcgcgggcgcggcgctgcagattccgtttgcgatgcagatggcgtatcgctttaacggcattggcggcggcggcggcagcggcaactttctgaccggcctgggccatcgcagcgatcattataactgcgtgagcagcggcggccagtgcctgtatagcgcgtgcccgatttttaccaaaattcagggcacctgctatcgcggcaaagcgaaatgctgcaaa

SEQ ID NO: 167 - DNA, 816 bp, synthetic construct
  misc_feature 1..816: note = SJ-S2'/FP1-2/AD Yeast (Saccharomyces) Optimized cDNA
  source 1..816: mol_type = other DNA
tctattattgcttatactatgtctttgggtgctgaaaattctgttgcttattctaataattctattgctattccaactaattttactatttctgttactactgaaattttgccagtttctatgactaaaacttctgttgattgtactatgtatatttgtggtgattctactgaatgttctaatttgttgttgcaatatggttctttttgtactcaattgaatagagctttgactggtattgctgttgaacaagataaaaatactcaagaagtttttgctcaagttaaacaaatttataaaactccaccaattaaagattttggtggttttaatttttctcaaattttgccagatccatctaaaccatctaaaagatcttttattgaagatttgttgtttaataaagttactttggctgatgctggttttattaaacaatatggtgattgtttgggtgatattgctgctagagatttgatttgtgctcaaaaatttaatggtttgactgttttgccaccattgttgactgatgaaatgattgctcaatatacttctgctttgttggctggtactattacttctggttggacttttggtgctggtgctgctttgcaaattccatttgctatgcaaatggcttatagatttaatggtattggtggtggtggtggttctggtaattttttgactggtttgggtcatagatctgatcattataattgtgtttcttctggtggtcaatgtttgtattctgcttgtccaatttttactaaaattcaaggtacttgttatagaggtaaagctaaatgttgtaaa

SEQ ID NO: 168 - protein, 4 aa, synthetic construct
  REGION 1..4: note = FCS3 motif
PRRA

SEQ ID NO: 169 - protein, 5 aa, synthetic construct
  REGION 1..5: note = G4S linker
GGGGS

SEQ ID NO: 170 - protein, 115 aa, synthetic construct
MASYQTQTNSPRRARSVASQSIIAYTMSLGAENGGGGSASYQTQTNSPRRARSVASQSIIAYTMSLGAENGGGGSASYQTQTNSPRRARSVASQSIIAYTMSLGAENLEHHHHHH

SEQ ID NO: 171 - protein, 239 aa, synthetic construct
MECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGALEHHHHHH